SEQUENCE LISTING (71 sequences)

SEQ ID NO: 1 - protein, 618 aa, Homo sapiens
MVSPRMSGLLSQTVILALIFLPQTRPAGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTSSCLSPRGPSSATTGCLVPGPGPCDGNPCANGGSCSETPRSFECTCPRGFYGLRCEVSGVTCADGPCFNGGLCVGGADPDSAYICHCPPGFQGSNCEKRVDRCSLQPCRNGGLCLDLGHALRCRCRAGFAGPRCEHDLDDCAGRACANGGTCVEGGGAHRCSCALGFGGRDCRERADPCAARPCAHGGRCYAHFSGLVCACAPGYMGARCEFPVHPDGASALPAAPPGLRPGDPQRYLLPPALGLLVAAGVAGAALLLVHVRRRGHSQDAGSRLLAGTPEPSVHALPDALNNLRTQEGSGDGPSSSVDWNRPEDVDPQGIYVISAPSIYAREVATPLFPPLHTGRAGQRQHLLFPYPSSILSVK

SEQ ID NO: 2 - protein, 585 aa, Mus musculus
MVSLQVSPLSQTLILAFLLPQALPAGVFELQIHSFGPGPGLGTPRSPCNARGPCRLFFRVCLKPGVSQEATESLCALGAALSTSVPVYTEHPGESAAALPLPDGLVRVPFRDAWPGTFSLVIETWREQLGEHAGGPAWNLLARVVGRRRLAAGGPWARDVQRTGTWELHFSYRARCEPPAVGAACARLCRSRSAPSRCGPGLRPCTPFPDECEAPSVCRPGCSPEHGYCEEPDECRCLEGWTGPLCTVPVSTSSCLNSRVPGPASTGCLLPGPGPCDGNPCANGGSCSETSGSFECACPRGFYGLRCEVSGVTCADGPCFNGGLCVGGEDPDSAYVCHCPPGFQGSNCEKRVDRCSLQPCQNGGLCLDLGHALRCRCRAGFAGPRCEHDLDDCAGRACANGGTCVEGGGSRRCSCALGFGGRDCRERADPCASRPCAHGGRCYAHFSGLVCACAPGYMGVRCEFAVRPDGADAVPAAPRGLRQADPQRFLLPPALGLLVAAGLAGAALLVIHVRRRGPGQDTGTRLLSGTREPSVHTLPDALNNLRLQDGAGDGPSSSADWNHPEDGDSRSIYVIPAPSIYAREA

SEQ ID NO: 3 - protein, 723 aa, Homo sapiens
MGSRCALALAVLSALLCQVWSSGVFELKLQEFVNKKGLLGNRNCCRGGAGPPPCACRTFFRVCLKHYQASVSPEPPCTYGSAVTPVLGVDSFSLPDGGGADSAFSNPIRFPFGFTWPGTFSLIIEALHTDSPDDLATENPERLISRLATQRHLTVGEEWSQDLHSSGRTDLKYSYRFVCDEHYYGEGCSVFCRPRDDAFGHFTCGERGEKVCNPGWKGPYCTEPICLPGCDEQHGFCDKPGECKCRVGWQGRYCDECIRYPGCLHGTCQQPWQCNCQEGWGGLFCNQDLNYCTHHKPCKNGATCTNTGQGSYTCSCRPGYTGATCELGIDECDPSPCKNGGSCTDLENSYSCTCPPGFYGKICELSAMTCADGPCFNGGRCSDSPDGGYSCRCPVGYSGFNCEKKIDYCSSSPCSNGAKCVDLGDAYLCRCQAGFSGRHCDDNVDDCASSPCANGGTCRDGVNDFSCTCPPGYTGRNCSAPVSRCEHAPCHNGATCHERGHRYVCECARGYGGPNCQFLLPELPPGPAVVDLTEKLEGQGGPFPWVAVCAGVILVLMLLLGCAAVVVCVRLRLQKHRPPADPCRGETETMNNLANCQREKDISVSIIGATQIKNTNKKADFHGDHSADKNGFKARYPAVDYNLVQDLKGDDTAVRDAHSKRDTKCQPQGSSGEEKGTPTTLRGGEASERKRPDSGCSTSKDTKYQSVYVISEEKDECVIATEV

SEQ ID NO: 4 - protein, 2555 aa, Homo sapiens
MPPLLAPLLCLALLPALAARGPRCSQPGETCLNGGKCEAANGTEACVCGGAFVGPRCQDPNPCLSTPCKNAGTCHVVDRRGVADYACSCALGFSGPLCLTPLDNACLTNPCRNGGTCDLLTLTEYKCRCPPGWSGKSCQQADPCASNPCANGGQCLPFEASYICHCPPSFHGPTCRQDVNECGQKPGLCRHGGTCHNEVGSYRCVCRATHTGPNCERPYVPCSPSPCQNGGTCRPTGDVTHECACLPGFTGQNCEENIDDCPGNNCKNGGACVDGVNTYNCRCPPEWTGQYCTEDVDECQLMPNACQNGGTCHNTHGGYNCVCVNGWTGEDCSENIDDCASAACFHGATCHDRVASFYCECPHGRTGLLCHLNDACISNPCNEGSNCDTNPVNGKAICTCPSGYTGPACSQDVDECSLGANPCEHAGKCINTLGSFECQCLQGYTGPRCEIDVNECVSNPCQNDATCLDQIGEFQCICMPGYEGVHCEVNTDECASSPCLHNGRCLDKINEFQCECPTGFTGHLCQYDVDECASTPCKNGAKCLDGPNTYTCVCTEGYTGTHCEVDIDECDPDPCHYGSCKDGVATFTCLCRPGYTGHHCETNINECSSQPCRHGGTCQDRDNAYLCFCLKGTTGPNCEINLDDCASSPCDSGTCLDKIDGYECACEPGYTGSMCNINIDECAGNPCHNGGTCEDGINGFTCRCPEGYHDPTCLSEVNECNSNPCVHGACRDSLNGYKCDCDPGWSGTNCDINNNECESNPCVNGGTCKDMTSGYVCTCREGFSGPNCQTNINECASNPCLNQGTCIDDVAGYKCNCLLPYTGATCEVVLAPCAPSPCRNGGECRQSEDYESFSCVCPTGWQGQTCEVDINECVLSPCRHGASCQNTHGGYRCHCQAGYSGRNCETDIDDCRPNPCHNGGSCTDGINTAFCDCLPGFRGTFCEEDINECASDPCRNGANCTDCVDSYTCTCPAGFSGIHCENNTPDCTESSCFNGGTCVDGINSFTCLCPPGFTGSYCQHDVNECDSQPCLHGGTCQDGCGSYRCTCPQGYTGPNCQNLVHWCDSSPCKNGGKCWQTHTQYRCECPSGWTGLYCDVPSVSCEVAAQRQGVDVARLCQHGGLCVDAGNTHHCRCQAGYTGSYCEDLVDECSPSPCQNGATCTDYLGGYSCKCVAGYHGVNCSEEIDECLSHPCQNGGTCLDLPNTYKCSCPRGTQGVHCEINVDDCNPPVDPVSRSPKCFNNGTCVDQVGGYSCTCPPGFVGERCEGDVNECLSNPCDARGTQNCVQRVNDFHCECRAGHTGRRCESVINGCKGKPCKNGGTCAVASNTARGFICKCPAGFEGATCENDARTCGSLRCLNGGTCISGPRSPTCLCLGPFTGPECQFPASSPCLGGNPCYNQGTCEPTSESPFYRCLCPAKFNGLLCHILDYSFGGGAGRDIPPPLIEEACELPECQEDAGNKVCSLQCNNHACGWDGGDCSLNFNDPWKNCTQSLQCWKYFSDGHCDSQCNSAGCLFDGFDCQRAEGQCNPLYDQYCKDHFSDGHCDQGCNSAECEWDGLDCAEHVPERLAAGTLVVVVLMPPEQLRNSSFHFLRELSRVLHTNVVFKRDAHGQQMIFPYYGREEELRKHPIKRAAEGWAAPDALLGQVKASLLPGGSEGGRRRRELDPMDVRGSIVYLEIDNRQCVQASSQCFQSATDVAAFLGALASLGSLNIPYKIEAVQSETVEPPPPAQLHFMYVAAAAFVLLFFVGCGVLLSRKRRRQHGQLWFPEGFKVSEASKKKRREPLGEDSVGLKPLKNASDGALMDDNQNEWGDEDLETKKFRFEEPVVLPDLDDQTDHRQWTQQHLDAADLRMSAMAPTPPQGEVDADCMDVNVRGPDGFTPLMIASCSGGGLETGNSEEEEDAPAVISDFIYQGASLHNQTDRTGETALHLAARYSRSDAAKRLLEASADANIQDNMGRTPLHAAVSADAQGVFQILIRNRATDLDARMHDGTTPLILAARLAVEGMLEDLINSHADVNAVDDLGKSALHWAAAVNNVDAAVVLLKNGANKDMQNNREETPLFLAAREGSYETAKVLLDHFANRDITDHMDRLPRDIAQERMHHDIVRLLDEYNLVRSPQLHGAPLGGTPTLSPPLCSPNGYLGSLKPGVQGKKVRKPSSKGLACGSKEAKDLKARRKKSQDGKGCLLDSSGMLSPVDSLESPHGYLSDVASPPLLPSPFQQSPSVPLNHLPGMPDTHLGIGHLNVAAKPEMAALGGGGRLAFETGPPRLSHLPVASGTSTVLGSSSGGALNFTVGGSTSLNGQCEWLSRLQSGMVPNQYNPLRGSVAPGPLSTQAPSLQHGMVGPLHSSLAASALSQMMSYQGLPSTRLATQPHLVQTQQVQPQNLQMQQQNLQPANIQQQQSLQPPPPPPQPHLGVSSAASGHLGRSFLSGEPSQADVQPLGPSSLAVHTILPQESPALPTSLPSSLVPPVTAAQFLTPPSQHSYSSPVDNTPSHQLQVPEHPFLTPSPESPDQWSSSSPHSNVSDWSEGVSSPPTSMQSQIARIPEAFK

SEQ ID NO: 5 - protein, 724 aa, synthetic construct
  REGION 1..724: note = human-mouse chimeric sequence
MVSPRMSGLLSQTVILALIFLPQTRPAGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTSSCLSPRGPSSATTGCLVPGPGPCDGNPCANGGSCSETPRSFECTCPRGFYGLRCEVSGVTCADGPCFNGGLCVGGADPDSAYICHCPPGFQGSNCEKRVDRCSLQPCRNGGLCLDLGHALRCRCRAGFAGPRCEHDLDDCAGRACANGGTCVEGGGAHRCSCALGFGGRDCRERADPCAARPCAHGGRCYAHFSGLVCACAPGYMGARCEFPVHPDGASALPAAPPGLRPGDPQRYLARGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 6 - protein, 575 aa, synthetic construct
  REGION 1..575: note = human-mouse chimeric sequence
MVLASSTTSIHTMLLLLLMLAQPAMAARCEPPAVGTACTRLCRPRSAPSRCGPGLRPCAPLEDECEAPLVCRAGCSPEHGFCEQPGECRCLEGWTGPLCTVPVSTSSCLSPRGPSSATTGCLVPGPGPCDGNPCANGGSCSETPRSFECTCPRGFYGLRCEVSGVTCADGPCFNGGLCVGGADPDSAYICHCPPGFQGSNCEKRVDRCSLQPCRNGGLCLDLGHALRCRCRAGFAGPRCEHDLDDCAGRACANGGTCVEGGGAHRCSCALGFGGRDCRERADPCAARPCAHGGRCYAHFSGLVCACAPGYMGARCEFPVHPDGASALPAAPPGLRPGDPQRYLARGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 7 - protein, 535 aa, synthetic construct
  REGION 1..535: note = human-mouse chimeric sequence
MVLASSTTSIHTMLLLLLMLAQPAMAAPLVCRAGCSPEHGFCEQPGECRCLEGWTGPLCTVPVSTSSCLSPRGPSSATTGCLVPGPGPCDGNPCANGGSCSETPRSFECTCPRGFYGLRCEVSGVTCADGPCFNGGLCVGGADPDSAYICHCPPGFQGSNCEKRVDRCSLQPCRNGGLCLDLGHALRCRCRAGFAGPRCEHDLDDCAGRACANGGTCVEGGGAHRCSCALGFGGRDCRERADPCAARPCAHGGRCYAHFSGLVCACAPGYMGARCEFPVHPDGASALPAAPPGLRPGDPQRYLARGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 8 - protein, 718 aa, synthetic construct
  REGION 1..718: note = human-mouse chimeric sequence
MVSLQVSPLSQTLILAFLLPQALPAGVFELQIHSFGPGPGLGTPRSPCNARGPCRLFFRVCLKPGVSQEATESLCALGAALSTSVPVYTEHPGESAAALPLPDGLVRVPFRDAWPGTFSLVIETWREQLGEHAGGPAWNLLARVVGRRRLAAGGPWARDVQRTGTWELHFSYRARCEPPAVGAACARLCRSRSAPSRCGPGLRPCTPFPDECEAPSVCRPGCSPEHGYCEEPDECRCLEGWTGPLCTVPVSTSSCLNSRVPGPASTGCLLPGPGPCDGNPCANGGSCSETSGSFECACPRGFYGLRCEVSGVTCADGPCFNGGLCVGGEDPDSAYVCHCPPGFQGSNCEKRVDRCSLQPCQNGGLCLDLGHALRCRCRAGFAGPRCEHDLDDCAGRACANGGTCVEGGGSRRCSCALGFGGRDCRERADPCASRPCAHGGRCYAHFSGLVCACAPGYMGVRCEFAVRPDGADAVPAAPRGLRQADPQRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 9 - protein, 121 aa, Mus musculus
QVQLQQSGADLVRPGTSVKVSCKASGYAFTNYLIEWIKQRPGQGLEWIGVMNPGSGGTHYSEKFRGKATLTADKSSSTAYMQLISLTSDDSAVYFCARSDYDYVTYAMDYWGQGTSVTVSS

SEQ ID NO: 10 - protein, 464 aa, Mus musculus
MEWSGVFIFLLSVTAGVHSQVQLQQSGADLVRPGTSVKVSCKASGYAFTNYLIEWIKQRPGQGLEWIGVMNPGSGGTHYSEKFRGKATLTADKSSSTAYMQLISLTSDDSAVYFCARSDYDYVTYAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 11 - protein, 470 aa, synthetic construct
  REGION 1..470: note = chimeric antibody heavy chain
MEWSGVFIFLLSVTAGVHSQVQLQQSGADLVRPGTSVKVSCKASGYAFTNYLIEWIKQRPGQGLEWIGVMNPGSGGTHYSEKFRGKATLTADKSSSTAYMQLISLTSDDSAVYFCARSDYDYVTYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 5 aa, Mus musculus
NYLIE

SEQ ID NO: 13 - protein, 17 aa, Mus musculus
VMNPGSGGTHYSEKFRG

SEQ ID NO: 14 - protein, 12 aa, Mus musculus
SDYDYVTYAMDY

SEQ ID NO: 15 - protein, 107 aa, Mus musculus
DIKMTQSPSSMYASLGERVTITCKASQDINSYLIWFQQKPGKSPKTLIYRTNRLVDGVPSRFSGSGSGQDYSLTISSLEYGDMGIYYCLQYDEFPFTFGSGTKLEIK

SEQ ID NO: 16 - protein, 236 aa, Mus musculus
MDMRTPAQFLGILLLWFPGIKCDIKMTQSPSSMYASLGERVTITCKASQDINSYLIWFQQKPGKSPKTLIYRTNRLVDGVPSRFSGSGSGQDYSLTISSLEYGDMGIYYCLQYDEFPFTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 17 - protein, 236 aa, synthetic construct
  REGION 1..236: note = chimeric antibody light chain
MDMRTPAQFLGILLLWFPGIKCDIKMTQSPSSMYASLGERVTITCKASQDINSYLIWFQQKPGKSPKTLIYRTNRLVDGVPSRFSGSGSGQDYSLTISSLEYGDMGIYYCLQYDEFPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 11 aa, Mus musculus
KASQDINSYLI

SEQ ID NO: 19 - protein, 7 aa, Mus musculus
RTNRLVD

SEQ ID NO: 20 - protein, 9 aa, Mus musculus
LQYDEFPFT

SEQ ID NO: 21 - protein, 118 aa, Mus musculus
EVQLQQSGPELVKPGASVKMSCRASGYTFTDYYMKWVKQSHGKSLEWIGDINPNNGDTFYNQKFKGKATLTIDKSSSTAYMQLNSLTSDDSAVYYCARDGNYAYFDYWGQGTTLTVSS

SEQ ID NO: 22 - protein, 461 aa, Mus musculus
MGWSWIFLFLLSGTGGVLSEVQLQQSGPELVKPGASVKMSCRASGYTFTDYYMKWVKQSHGKSLEWIGDINPNNGDTFYNQKFKGKATLTIDKSSSTAYMQLNSLTSDDSAVYYCARDGNYAYFDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 23 - protein, 467 aa, synthetic construct
  REGION 1..467: note = chimeric antibody heavy chain
MGWSWIFLFLLSGTGGVLSEVQLQQSGPELVKPGASVKMSCRASGYTFTDYYMKWVKQSHGKSLEWIGDINPNNGDTFYNQKFKGKATLTIDKSSSTAYMQLNSLTSDDSAVYYCARDGNYAYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 5 aa, Mus musculus
DYYMK

SEQ ID NO: 25 - protein, 17 aa, Mus musculus
DINPNNGDTFYNQKFKG

SEQ ID NO: 26 - protein, 9 aa, Mus musculus
DGNYAYFDY

SEQ ID NO: 27 - protein, 111 aa, Mus musculus
DIVLTQSPASLAVSLGQRATISCRASKSVSTSGYSYMHWYQQKPGQPPKLLIFLASNLESGVPARFSGSGSGTDFTLNIHPVEEEDAATYYCQHSRHLPWTFGGGTKLEIK

SEQ ID NO: 28 - protein, 238 aa, Mus musculus
METDTLLLWVLLLWVPGSTGDIVLTQSPASLAVSLGQRATISCRASKSVSTSGYSYMHWYQQKPGQPPKLLIFLASNLESGVPARFSGSGSGTDFTLNIHPVEEEDAATYYCQHSRHLPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 29 - protein, 238 aa, synthetic construct
  REGION 1..238: note = chimeric antibody light chain
METDTLLLWVLLLWVPGSTGDIVLTQSPASLAVSLGQRATISCRASKSVSTSGYSYMHWYQQKPGQPPKLLIFLASNLESGVPARFSGSGSGTDFTLNIHPVEEEDAATYYCQHSRHLPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 15 aa, Mus musculus
RASKSVSTSGYSYMH

SEQ ID NO: 31 - protein, 7 aa, Mus musculus
LASNLES

SEQ ID NO: 32 - protein, 9 aa, Mus musculus
QHSRHLPWT

SEQ ID NO: 33 - protein, 119 aa, Mus musculus
QVQLQQSGGDLMKPGASVKISCKAAGYTFSNYYIEWVKQRPGHGLEWIGEILPGSGSTTYNEKFKGKASFTADTSSNTAYMQLSSLTSEDSAVYYCARWGAREPGFPYWGQGTLVTVSA

SEQ ID NO: 34 - protein, 462 aa, Mus musculus
MEWTWVFLFLLSVTAGVHSQVQLQQSGGDLMKPGASVKISCKAAGYTFSNYYIEWVKQRPGHGLEWIGEILPGSGSTTYNEKFKGKASFTADTSSNTAYMQLSSLTSEDSAVYYCARWGAREPGFPYWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 35 - protein, 468 aa, synthetic construct
  REGION 1..468: note = chimeric antibody heavy chain
MEWTWVFLFLLSVTAGVHSQVQLQQSGGDLMKPGASVKISCKAAGYTFSNYYIEWVKQRPGHGLEWIGEILPGSGSTTYNEKFKGKASFTADTSSNTAYMQLSSLTSEDSAVYYCARWGAREPGFPYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 5 aa, Mus musculus
NYYIE

SEQ ID NO: 37 - protein, 17 aa, Mus musculus
EILPGSGSTTYNEKFKG

SEQ ID NO: 38 - protein, 10 aa, Mus musculus
WGAREPGFPY

SEQ ID NO: 39 - protein, 107 aa, Mus musculus
DIVMTQSQKFMSTSVGDRVSVTCKASQNVGTNVAWYQQKPGQSPKALIYSASYRYSGVPDRFTGSGSGTDFTLTISNVQSEDLAEYFCQQYNNYPLTFGAGTKLELK

SEQ ID NO: 40 - protein, 238 aa, Mus musculus
MGIKMESQTQVFVYMLLWLSGVDGDIVMTQSQKFMSTSVGDRVSVTCKASQNVGTNVAWYQQKPGQSPKALIYSASYRYSGVPDRFTGSGSGTDFTLTISNVQSEDLAEYFCQQYNNYPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 41 - protein, 238 aa, synthetic construct
  REGION 1..238: note = chimeric antibody light chain
MGIKMESQTQVFVYMLLWLSGVDGDIVMTQSQKFMSTSVGDRVSVTCKASQNVGTNVAWYQQKPGQSPKALIYSASYRYSGVPDRFTGSGSGTDFTLTISNVQSEDLAEYFCQQYNNYPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - protein, 11 aa, Mus musculus
KASQNVGTNVA

SEQ ID NO: 43 - protein, 7 aa, Mus musculus
SASYRYS

SEQ ID NO: 44 - protein, 9 aa, Mus musculus
QQYNNYPLT

SEQ ID NO: 45 - protein, 123 aa, Mus musculus
EVKLVESGGGLVQPGGSLSLSCAASGFTFTDYYMNWVRQPPGKALEWLALIRNKANGYTTEYNASVKGRFTISRDNSQNILYLQMNALRAEDSATYYCARDSDGYYEYYFDYWGQGTTLTVSS

SEQ ID NO: 46 - protein, 466 aa, Mus musculus
MKLWLNWIFLVTLLNGIQCEVKLVESGGGLVQPGGSLSLSCAASGFTFTDYYMNWVRQPPGKALEWLALIRNKANGYTTEYNASVKGRFTISRDNSQNILYLQMNALRAEDSATYYCARDSDGYYEYYFDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 47 - protein, 472 aa, synthetic construct [sequence omitted]

SEQ ID NO: 48 - protein, 5 aa, Mus musculus
DYYMN

SEQ ID NO: 49 - protein, 19 aa, Mus musculus
LIRNKANGYTTEYNASVKG

SEQ ID NO: 50 - protein, 12 aa, Mus musculus
DSDGYYEYYFDY

SEQ ID NO: 51 - protein, 107 aa, Mus musculus
DIQMTQSPSSLSASLGERVSLTCRASQEISDYLSWLQQKPDGTIKRLIFAASTLDSGVPKRFSGSRSGSDFSLSISSLESEDFADYYCLQYASYPYTFGSGTKLEIK

SEQ ID NO: 52 - protein, 236 aa, Mus musculus
MDMRVPAHVFGFLLLWFPGTRCDIQMTQSPSSLSASLGERVSLTCRASQEISDYLSWLQQKPDGTIKRLIFAASTLDSGVPKRFSGSRSGSDFSLSISSLESEDFADYYCLQYASYPYTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 53 - protein, 236 aa, synthetic construct
  REGION 1..236: note = chimeric antibody light chain
MDMRVPAHVFGFLLLWFPGTRCDIQMTQSPSSLSASLGERVSLTCRASQEISDYLSWLQQKPDGTIKRLIFAASTLDSGVPKRFSGSRSGSDFSLSISSLESEDFADYYCLQYASYPYTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - protein, 11 aa, Mus musculus
RASQEISDYLS

SEQ ID NO: 55 - protein, 7 aa, Mus musculus
AASTLDS

SEQ ID NO: 56 - protein, 9 aa, Mus musculus
LQYASYPYT

SEQ ID NO: 57 - DNA, 1857 bp, Homo sapiens
  source 1..1857: mol_type = other DNA
atggtctccccacggatgtccgggctcctctcccagactgtgatcctagcgctcattttcctcccccagacacggcccgctggcgtcttcgagctgcagatccactctttcgggccgggtccaggccctggggccccgcggtccccctgcagcgcccggctcccctgccgcctcttcttcagagtctgcctgaagcctgggctctcagaggaggccgccgagtccccgtgcgccctgggcgcggcgctgagtgcgcgcggaccggtctacaccgagcagcccggagcgcccgcgcctgatctcccactgcccgacggcctcttgcaggtgcccttccgggacgcctggcctggcaccttctctttcatcatcgaaacctggagagaggagttaggagaccagattggagggcccgcctggagcctgctggcgcgcgtggctggcaggcggcgcttggcagccggaggcccgtgggcccgggacattcagcgcgcaggcgcctgggagctgcgcttctcgtaccgcgcgcgctgcgagccgcctgccgtcgggaccgcgtgcacgcgcctctgccgtccgcgcagcgccccctcgcggtgcggtccgggactgcgcccctgcgcaccgctcgaggacgaatgtgaggcgccgctggtgtgccgagcaggctgcagccctgagcatggcttctgtgaacagcccggtgaatgccgatgcctagagggctggactggacccctctgcacggtccctgtctccaccagcagctgcctcagccccaggggcccgtcctctgctaccaccggatgccttgtccctgggcctgggccctgtgacgggaacccgtgtgccaatggaggcagctgtagtgagacacccaggtcctttgaatgcacctgcccgcgtgggttctacgggctgcggtgtgaggtgagcggggtgacatgtgcagatggaccctgcttcaacggcggcttgtgtgtcgggggtgcagaccctgactctgcctacatctgccactgcccacccggtttccaaggctccaactgtgagaagagggtggaccggtgcagcctgcagccatgccgcaatggcggactctgcctggacctgggccacgccctgcgctgccgctgccgcgccggcttcgcgggtcctcgctgcgagcacgacctggacgactgcgcgggccgcgcctgcgctaacggcggcacgtgtgtggagggcggcggcgcgcaccgctgctcctgcgcgctgggcttcggcggccgcgactgccgcgagcgcgcggacccgtgcgccgcgcgcccctgtgctcacggcggccgctgctacgcccacttctccggcctcgtctgcgcttgcgctcccggctacatgggagcgcggtgtgagttcccagtgcaccccgacggcgcaagcgccttgcccgcggccccgccgggcctcaggcccggggaccctcagcgctaccttttgcctccggctctgggactgctcgtggccgcgggcgtggccggcgctgcgctcttgctggtccacgtgcgccgccgtggccactcccaggatgctgggtctcgcttgctggctgggaccccggagccgtcagtccacgcactcccggatgcactcaacaacctaaggacgcaggagggttccggggatggtccgagctcgtccgtagattggaatcgccctgaagatgtagaccctcaagggatttatgtcatatctgctccttccatctacgctcgggaggtagcgacgccccttttccccccgctacacactgggcgcgctgggcagaggcagcacctgctttttccctacccttcctcgattctgtccgtgaaatga

SEQ ID NO: 58 - DNA, 1758 bp, Mus musculus
  source 1..1758: mol_type = other DNA
atggtctctctgcaggtgtctccgctttcccagacgctgatcctggcttttcttcttcctcaggcactgccagctggtgtcttcgagctacaaattcattctttcgggccaggcccaggcctcgggaccccacgctccccctgcaacgcccgaggcccttgccgcctcttcttcagggtctgcctgaagcccggagtctcccaggaggccaccgagtccctgtgcgccctgggcgcagcactgagcacgagcgtcccggtctatacggagcaccccggagagtcagcggctgccctgccgctgcctgatggcctcgtacgtgtgcccttccgcgatgcttggccgggcaccttctccctcgtcattgaaacctggagagagcagctgggagagcatgctggagggcccgcctggaacctgctagcacgtgtggtcggccgtagacgcctggcggctgggggcccgtgggcccgcgatgtgcagcgcacaggcacatgggagttgcacttctcctaccgcgcgcggtgcgagccgcccgccgtcggggccgcctgcgcgcgcctgtgccgctcacgcagtgccccctcgcggtgtggcccgggactgcgaccctgcacgccattcccagacgagtgcgaagccccgtctgtgtgtcgaccaggctgcagccccgagcacggctactgtgaagagcctgatgaatgccgttgcctggagggctggactggacccctctgcacggtccctgtctccaccagtagctgcctgaactccagggttcctggtcctgccagcactggatgccttttacctgggcctggaccttgtgatgggaacccatgtgccaatgggggcagctgtagtgaaacctctggctcctttgaatgtgcctgtccccggggattctacgggcttcgatgtgaggtgagcggggtcacgtgcgcagatggaccctgcttcaatggcggcttgtgtgttggcggtgaagatcctgactctgcctatgtctgtcattgcccacctggtttccaaggctctaactgtgagaagagggtggaccgctgtagcctgcagccatgtcagaatggcggcctctgcctggacctgggccacgcgttgcgctgccgctgtcgcgcgggattcgccgggccgcgctgcgagcacgacctggacgactgcgccggccgcgcctgtgccaacggcggcacgtgcgtggagggcggcggctcgcgccgctgctcctgtgcgctgggcttcggcgggcgcgactgccgagaacgcgccgacccctgcgcctcccgcccctgcgcgcatggaggccgttgctacgcccacttctctggcctggtctgcgcctgcgcgcccggctacatgggcgtgagatgcgagttcgctgtgcgcccggacggcgcggacgcggtgcccgccgccccgcggggcctgaggcaggcggatccacagcgctttcttctgcctcccgccttggggctgctggtggccgccggtttggctggcgccgcactcttggtcatccacgttcgccgccgaggtcctggccaggataccgggactcgcctgctttctgggacccgggagccttcggtccacacgctcccggatgcactcaacaacctgaggttacaagacggtgctggggatggccccagttcgtcggctgactggaatcatcctgaagatggagactctagatccatttatgtcataccagccccttccatttatgcacgagaggcctga

SEQ ID NO: 59 - DNA, 2172 bp, Homo sapiens
  source 1..2172: mol_type = other DNA
atgggcagtcggtgcgcgctggccctggcggtgctctcggccttgctgtgtcaggtctggagctctggggtgttcgaactgaagctgcaggagttcgtcaacaagaaggggctgctggggaaccgcaactgctgccgcgggggcgcggggccaccgccgtgcgcctgccggaccttcttccgcgtgtgcctcaagcactaccaggccagcgtgtcccccgagccgccctgcacctacggcagcgccgtcacccccgtgctgggcgtcgactccttcagtctgcccgacggcgggggcgccgactccgcgttcagcaaccccatccgcttccccttcggcttcacctggccgggcaccttctctctgattattgaagctctccacacagattctcctgatgacctcgcaacagaaaacccagaaagactcatcagccgcctggccacccagaggcacctgacggtgggcgaggagtggtcccaggacctgcacagcagcggccgcacggacctcaagtactcctaccgcttcgtgtgtgacgaacactactacggagagggctgctccgttttctgccgtccccgggacgatgccttcggccacttcacctgtggggagcgtggggagaaagtgtgcaaccctggctggaaagggccctactgcacagagccgatctgcctgcctggatgtgatgagcagcatggattttgtgacaaaccaggggaatgcaagtgcagagtgggctggcagggccggtactgtgacgagtgtatccgctatccaggctgtctccatggcacctgccagcagccctggcagtgcaactgccaggaaggctgggggggccttttctgcaaccaggacctgaactactgcacacaccataagccctgcaagaatggagccacctgcaccaacacgggccaggggagctacacttgctcttgccggcctgggtacacaggtgccacctgcgagctggggattgacgagtgtgaccccagcccttgtaagaacggagggagctgcacggatctcgagaacagctactcctgtacctgcccacccggcttctacggcaaaatctgtgaattgagtgccatgacctgtgcggacggcccttgctttaacgggggtcggtgctcagacagccccgatggagggtacagctgccgctgccccgtgggctactccggcttcaactgtgagaagaaaattgactactgcagctcttcaccctgttctaatggtgccaagtgtgtggacctcggtgatgcctacctgtgccgctgccaggccggcttctcggggaggcactgtgacgacaacgtggacgactgcgcctcctccccgtgcgccaacgggggcacctgccgggatggcgtgaacgacttctcctgcacctgcccgcctggctacacgggcaggaactgcagtgcccccgtcagcaggtgcgagcacgcaccctgccacaatggggccacctgccacgagaggggccaccgctatgtgtgcgagtgtgcccgaggctacgggggtcccaactgccagttcctgctccccgagctgcccccgggcccagcggtggtggacctcactgagaagctagagggccagggcgggccattcccctgggtggccgtgtgcgccggggtcatccttgtcctcatgctgctgctgggctgtgccgctgtggtggtctgcgtccggctgaggctgcagaagcaccggcccccagccgacccctgccggggggagacggagaccatgaacaacctggccaactgccagcgtgagaaggacatctcagtcagcatcatcggggccacgcagatcaagaacaccaacaagaaggcggacttccacggggaccacagcgccgacaagaatggcttcaaggcccgctacccagcggtggactataacctcgtgcaggacctcaagggtgacgacaccgccgtcagggacgcgcacagcaagcgtgacaccaagtgccagccccagggctcctcaggggaggagaaggggaccccgaccacactcaggggtggagaagcatctgaaagaaaaaggccggactcgggctgttcaacttcaaaagacaccaagtaccagtcggtgtacgtcatatccgaggagaaggatgagtgcgtcatagcaactgaggtgtaa

SEQ ID NO: 60 - DNA, 7668 bp, Homo sapiens
  source 1..7668: mol_type = other DNA
atgccgccgctcctggcgcccctgctctgcctggcgctgctgcccgcgctcgccgcacgaggcccgcgatgctcccagcccggtgagacctgcctgaatggcgggaagtgtgaagcggccaatggcacggaggcctgcgtctgtggcggggccttcgtgggcccgcgatgccaggaccccaacccgtgcctcagcaccccctgcaagaacgccgggacatgccacgtggtggaccgcagaggcgtggcagactatgcctgcagctgtgccctgggcttctctgggcccctctgcctgacacccctggacaatgcctgcctcaccaacccctgccgcaacgggggcacctgcgacctgctcacgctgacggagtacaagtgccgctgcccgcccggctggtcagggaaatcgtgccagcaggctgacccgtgcgcctccaacccctgcgccaacggtggccagtgcctgcccttcgaggcctcctacatctgccactgcccacccagcttccatggccccacctgccggcaggatgtcaacgagtgtggccagaagcccgggctttgccgccacggaggcacctgccacaacgaggtcggctcctaccgctgcgtctgccgcgccacccacactggccccaactgcgagcggccctacgtgccctgcagcccctcgccctgccagaacgggggcacctgccgccccacgggcgacgtcacccacgagtgtgcctgcctgccaggcttcaccggccagaactgtgaggaaaatatcgacgattgtccaggaaacaactgcaagaacgggggtgcctgtgtggacggcgtgaacacctacaactgccgctgcccgccagagtggacaggtcagtactgtaccgaggatgtggacgagtgccagctgatgccaaatgcctgccagaacggcgggacctgccacaacacccacggtggctacaactgcgtgtgtgtcaacggctggactggtgaggactgcagcgagaacattgatgactgtgccagcgccgcctgcttccacggcgccacctgccatgaccgtgtggcctccttctactgcgagtgtccccatggccgcacaggtctgctgtgccacctcaacgacgcatgcatcagcaacccctgtaacgagggctccaactgcgacaccaaccctgtcaatggcaaggccatctgcacctgcccctcggggtacacgggcccggcctgcagccaggacgtggatgagtgctcgctgggtgccaacccctgcgagcatgcgggcaagtgcatcaacacgctgggctccttcgagtgccagtgtctgcagggctacacgggcccccgatgcgagatcgacgtcaacgagtgcgtctcgaacccgtgccagaacgacgccacctgcctggaccagattggggagttccagtgcatctgcatgcccggctacgagggtgtgcactgcgaggtcaacacagacgagtgtgccagcagcccctgcctgcacaatggccgctgcctggacaagatcaatgagttccagtgcgagtgccccacgggcttcactgggcatctgtgccagtacgatgtggacgagtgtgccagcaccccctgcaagaatggtgccaagtgcctggacggacccaacacttacacctgtgtgtgcacggaagggtacacggggacgcactgcgaggtggacatcgatgagtgcgaccccgacccctgccactacggctcctgcaaggacggcgtcgccaccttcacctgcctctgccgcccaggctacacgggccaccactgcgagaccaacatcaacgagtgctccagccagccctgccgccacgggggcacctgccaggaccgcgacaacgcctacctctgcttctgcctgaaggggaccacaggacccaactgcgagatcaacctggatgactgtgccagcagcccctgcgactcgggcacctgtctggacaagatcgatggctacgagtgtgcctgtgagccgggctacacagggagcatgtgtaacatcaacatcgatgagtgtgcgggcaacccctgccacaacgggggcacctgcgaggacggcatcaatggcttcacctgccgctgccccgagggctaccacgaccccacctgcctgtctgaggtcaatgagtgcaacagcaacccctgcgtccacggggcctgccgggacagcctcaacgggtacaagtgcgactgtgaccctgggtggagtgggaccaactgtgacatcaacaacaatgagtgtgaatccaacccttgtgtcaacggcggcacctgcaaagacatgaccagtggctacgtgtgcacctgccgggagggcttcagcggtcccaactgccagaccaacatcaacgagtgtgcgtccaacccatgtctgaaccagggcacgtgtattgacgacgttgccgggtacaagtgcaactgcctgctgccctacacaggtgccacgtgtgaggtggtgctggccccgtgtgcccccagcccctgcagaaacggcggggagtgcaggcaatccgaggactatgagagcttctcctgtgtctgccccacgggctggcaagggcagacctgtgaggtcgacatcaacgagtgcgttctgagcccgtgccggcacggcgcatcctgccagaacacccacggcggctaccgctgccactgccaggccggctacagtgggcgcaactgcgagaccgacatcgacgactgccggcccaacccgtgtcacaacgggggctcctgcacagacggcatcaacacggccttctgcgactgcctgcccggcttccggggcactttctgtgaggaggacatcaacgagtgtgccagtgacccctgccgcaacggggccaactgcacggactgcgtggacagctacacgtgcacctgccccgcaggcttcagcgggatccactgtgagaacaacacgcctgactgcacagagagctcctgcttcaacggtggcacctgcgtggacggcatcaactcgttcacctgcctgtgtccacccggcttcacgggcagctactgccagcacgatgtcaatgagtgcgactcacagccctgcctgcatggcggcacctgtcaggacggctgcggctcctacaggtgcacctgcccccagggctacactggccccaactgccagaaccttgtgcactggtgtgactcctcgccctgcaagaacggcggcaaatgctggcagacccacacccagtaccgctgcgagtgccccagcggctggaccggcctttactgcgacgtgcccagcgtgtcctgtgaggtggctgcgcagcgacaaggtgttgacgttgcccgcctgtgccagcatggagggctctgtgtggacgcgggcaacacgcaccactgccgctgccaggcgggctacacaggcagctactgtgaggacctggtggacgagtgctcacccagcccctgccagaacggggccacctgcacggactacctgggcggctactcctgcaagtgcgtggccggctaccacggggtgaactgctctgaggagatcgacgagtgcctctcccacccctgccagaacgggggcacctgcctcgacctccccaacacctacaagtgctcctgcccacggggcactcagggtgtgcactgtgagatcaacgtggacgactgcaatccccccgttgaccccgtgtcccggagccccaagtgctttaacaacggcacctgcgtggaccaggtgggcggctacagctgcacctgcccgccgggcttcgtgggtgagcgctgtgagggggatgtcaacgagtgcctgtccaatccctgcgacgcccgtggcacccagaactgcgtgcagcgcgtcaatgacttccactgcgagtgccgtgctggtcacaccgggcgccgctgcgagtccgtcatcaatggctgcaaaggcaagccctgcaagaatgggggcacctgcgccgtggcctccaacaccgcccgcgggttcatctgcaagtgccctgcgggcttcgagggcgccacgtgtgagaatgacgctcgtacctgcggcagcctgcgctgcctcaacggcggcacatgcatctccggcccgcgcagccccacctgcctgtgcctgggccccttcacgggccccgaatgccagttcccggccagcagcccctgcctgggcggcaacccctgctacaaccaggggacctgtgagcccacatccgagagccccttctaccgttgcctgtgccccgccaaattcaacgggctcttgtgccacatcctggactacagcttcgggggtggggccgggcgcgacatccccccgccgctgatcgaggaggcgtgcgagctgcccgagtgccaggaggacgcgggcaacaaggtctgcagcctgcagtgcaacaaccacgcgtgcggctgggacggcggtgactgctccctcaacttcaatgacccctggaagaactgcacgcagtctctgcagtgctggaagtacttcagtgacggccactgtgacagccagtgcaactcagccggctgcctcttcgacggctttgactgccagcgtgcggaaggccagtgcaaccccctgtacgaccagtactgcaaggaccacttcagcgacgggcactgcgaccagggctgcaacagcgcggagtgcgagtgggacgggctggactgtgcggagcatgtacccgagaggctggcggccggcacgctggtggtggtggtgctgatgccgccggagcagctgcgcaacagctccttccacttcctgcgggagctcagccgcgtgctgcacaccaacgtggtcttcaagcgtgacgcacacggccagcagatgatcttcccctactacggccgcgaggaggagctgcgcaagcaccccatcaagcgtgccgccgagggctgggccgcacctgacgccctgctgggccaggtgaaggcctcgctgctccctggtggcagcgagggtgggcggcggcggagggagctggaccccatggacgtccgcggctccatcgtctacctggagattgacaaccggcagtgtgtgcaggcctcctcgcagtgcttccagagtgccaccgacgtggccgcattcctgggagcgctcgcctcgctgggcagcctcaacatcccctacaagatcgaggccgtgcagagtgagaccgtggagccgcccccgccggcgcagctgcacttcatgtacgtggcggcggccgcctttgtgcttctgttcttcgtgggctgcggggtgctgctgtcccgcaagcgccggcggcagcatggccagctctggttccctgagggcttcaaagtgtctgaggccagcaagaagaagcggcgggagcccctcggcgaggactccgtgggcctcaagcccctgaagaacgcttcagacggtgccctcatggacgacaaccagaatgagtggggggacgaggacctggagaccaagaagttccggttcgaggagcccgtggttctgcctgacctggacgaccagacagaccaccggcagtggactcagcagcacctggatgccgctgacctgcgcatgtctgccatggcccccacaccgccccagggtgaggttgacgccgactgcatggacgtcaatgtccgcgggcctgatggcttcaccccgctcatgatcgcctcctgcagcgggggcggcctggagacgggcaacagcgaggaagaggaggacgcgccggccgtcatctccgacttcatctaccagggcgccagcctgcacaaccagacagaccgcacgggcgagaccgccttgcacctggccgcccgctactcacgctctgatgccgccaagcgcctgctggaggccagcgcagatgccaacatccaggacaacatgggccgcaccccgctgcatgcggctgtgtctgccgacgcacaaggtgtcttccagatcctgatccggaaccgagccacagacctggatgcccgcatgcatgatggcacgacgccactgatcctggctgcccgcctggccgtggagggcatgctggaggacctcatcaactcacacgccgacgtcaacgccgtagatgacctgggcaagtccgccctgcactgggccgccgccgtgaacaatgtggatgccgcagttgtgctcctgaagaacggggctaacaaagatatgcagaacaacagggaggagacacccctgtttctggccgcccgggagggcagctacgagaccgccaaggtgctgctggaccactttgccaaccgggacatcacggatcatatggaccgcctgccgcgcgacatcgcacaggagcgcatgcatcacgacatcgtgaggctgctggacgagtacaacctggtgcgcagcccgcagctgcacggagccccgctggggggcacgcccaccctgtcgcccccgctctgctcgcccaacggctacctgggcagcctcaagcccggcgtgcagggcaagaaggtccgcaagcccagcagcaaaggcctggcctgtggaagcaaggaggccaaggacctcaaggcacggaggaagaagtcccaggacggcaagggctgcctgctggacagctccggcatgctctcgcccgtggactccctggagtcaccccatggctacctgtcagacgtggcctcgccgccactgctgccctccccgttccagcagtctccgtccgtgcccctcaaccacctgcctgggatgcccgacacccacctgggcatcgggcacctgaacgtggcggccaagcccgagatggcggcgctgggtgggggcggccggctggcctttgagactggcccacctcgtctctcccacctgcctgtggcctctggcaccagcaccgtcctgggctccagcagcggaggggccctgaatttcactgtgggcgggtccaccagtttgaatggtcaatgcgagtggctgtcccggctgcagagcggcatggtgccgaaccaatacaaccctctgcgggggagtgtggcaccaggccccctgagcacacaggccccctccctgcagcatggcatggtaggcccgctgcacagtagccttgctgccagcgccctgtcccagatgatgagctaccagggcctgcccagcacccggctggccacccagcctcacctggtgcagacccagcaggtgcagccacaaaacttacagatgcagcagcagaacctgcagccagcaaacatccagcagcagcaaagcctgcagccgccaccaccaccaccacagccgcaccttggcgtgagctcagcagccagcggccacctgggccggagcttcctgagtggagagccgagccaggcagacgtgcagccactgggccccagcagcctggcggtgcacactattctgccccaggagagccccgccctgcccacgtcgctgccatcctcgctggtcccacccgtgaccgcagcccagttcctgacgcccccctcgcagcacagctactcctcgcctgtggacaacacccccagccaccagctacaggtgcctgagcaccccttcctcaccccgtcccctgagtcccctgaccagtggtccagctcgtccccgcattccaacgtctccgactggtccgagggcgtctccagccctcccaccagcatgcagtcccagatcgcccgcattccggaggccttcaagtaa

SEQ ID NO: 61 - protein, 4 aa, synthetic construct
  REGION 1..4: note = artificial linker
GGGS

SEQ ID NO: 62 - protein, 4 aa, synthetic construct
  REGION 1..4: note = artificial linker
SGGG

SEQ ID NO: 63 - protein, 5 aa, synthetic construct
  REGION 1..5: note = artificial linker
GGGGS

SEQ ID NO: 64 - protein, 5 aa, synthetic construct
  REGION 1..5: note = artificial linker
SGGGG

SEQ ID NO: 65 - protein, 6 aa, synthetic construct
  REGION 1..6: note = artificial linker
GGGGGS

SEQ ID NO: 66 - protein, 6 aa, synthetic construct
  REGION 1..6: note = artificial linker
SGGGGG

SEQ ID NO: 67 - protein, 7 aa, synthetic construct
  REGION 1..7: note = artificial linker
GGGGGGS

SEQ ID NO: 68 - protein, 7 aa, synthetic construct
  REGION 1..7: note = artificial linker
SGGGGGG

SEQ ID NO: 69 - protein, 475 aa, Mus musculus
MEWSGVFIFLLSVTAGVHSQVQLQQSGADLVRPGTSVKVSCKASGYAFTNYLIEWIKQRPGQGLEWIGVMNPGSGGTHYSEKFRGKATLTADKSSSTAYMQLISLTSDDSAVYFCARSDYDYVTYAMDYWGQGTSVTVSSAKTTAPSVYPLAPVCGGTTGSSVTLGCLVKGYFPEPVTLTWNSGSLSSGVHTFPALLQSGLYTLSSSVTVTSNTWPSQTITCNVAHPASSTKVDKKIEPRVPITQNPCPPLKECPPCAAPDLLGGPSVFIFPPKIKDVLMISLSPMVTCVVVDVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLGK

SEQ ID NO: 70 - protein, 472 aa, Mus musculus
MGWSWIFLFLLSGTGGVLSEVQLQQSGPELVKPGASVKMSCRASGYTFTDYYMKWVKQSHGKSLEWIGDINPNNGDTFYNQKFKGKATLTIDKSSSTAYMQLNSLTSDDSAVYYCARDGNYAYFDYWGQGTTLTVSSAKTTAPSVYPLAPVCGGTTGSSVTLGCLVKGYFPEPVTLTWNSGSLSSGVHTFPALLQSGLYTLSSSVTVTSNTWPSQTITCNVAHPASSTKVDKKIEPRVPITQNPCPPLKECPPCAAPDLLGGPSVFIFPPKIKDVLMISLSPMVTCVVVDVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLGK

SEQ ID NO: 71 - protein, 477 aa, Mus musculus
MKLWLNWIFLVTLLNGIQCEVKLVESGGGLVQPGGSLSLSCAASGFTFTDYYMNWVRQPPGKALEWLALIRNKANGYTTEYNASVKGRFTISRDNSQNILYLQMNALRAEDSATYYCARDSDGYYEYYFDYWGQGTTLTVSSAKTTAPSVYPLAPVCGGTTGSSVTLGCLVKGYFPEPVTLTWNSGSLSSGVHTFPALLQSGLYTLSSSVTVTSNTWPSQTITCNVAHPASSTKVDKKIEPRVPITQNPCPPLKECPPCAAPDLLGGPSVFIFPPKIKDVLMISLSPMVTCVVVDVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLGK

(19) United States
(12) Patent Application Publication
(10) Pub. No.: US 2025/0236679 A1
(43) Pub. Date: Jul. 24, 2025

(54) ANTI-DLL3 ANTIBODY

(71) Applicants: CHUGAI SEIYAKU KABUSHIKI KAISHA, Tokyo (JP); THE UNIVERSITY OF TOKYO, Tokyo (JP)

(72) Inventors: Kenji YOSHIDA, Tokyo (JP); Hiroyuki ABURATANI, Tokyo (JP); Shumpei ISHIKAWA, Tokyo (JP)

(73) Assignees: CHUGAI SEIYAKU KABUSHIKI KAISHA, Tokyo (JP); THE UNIVERSITY OF TOKYO, Tokyo (JP)

(21) Appl. No.: 19/077,985

(22) Filed: Mar. 12, 2025

Related U.S. Application Data
(63) Continuation of application No. 18/782,248, filed on Jul. 24, 2024, now abandoned.
Application No. 18/782,248 is a continuation of application No. 17/406,504, filed on Aug. 19, 2021, now abandoned.
Application No. 17/406,504 is a continuation of application No. 14/846,135, filed on Sep. 4, 2015, now Pat. No. 11,111,311.
Application No. 14/846,135 is a continuation of application No. 13/575,861, filed on Sep. 12, 2012, now Pat. No. 9,127,071.

(30) Foreign Application Priority Data
Jan. 29, 2010 (JP) 2010-019391

(51) Int. Cl. C07K 16/30; A61K 39/00; A61K 39/395; C07K 16/28; C12Q 1/6886; G01N 33/574

(52) U.S. Cl. CPC C07K 16/3023; A61K 39/395; C07K 16/28; C12Q 1/6886; G01N 33/574; A61K 2039/505; C07K 2317/24; C07K 2317/30; C07K 2317/732; C07K 2317/77; C07K 2319/30; C12Q 2600/118; G01N 2333/4703

(57) ABSTRACT

An antibody which binds to DLL3 protein, composition containing the antibody, and uses thereof are disclosed. The antibody recognizes a region from amino acids 216 to 492 in human DLL3 having the amino acid sequence as set forth in SEQ ID NO: 1. Also disclosed are a pharmaceutical composition, for example, an anticancer agent, containing the antibody of the present invention as an active ingredient. Further disclosed is a method for diagnosing cancer using the antibody and a diagnostic drug for cancer containing the antibody.

### REFERENCE TO RELATED APPLICATIONS

The present application is a Continuation of U.S. Application Ser. No. 18/782,248, filed Jul. 24, 2024, which is a Continuation of U.S. application Ser. No. 17/406,504, filed Aug. 19, 2021, which is a Continuation of U.S. application Ser. No. 14/846,135, filed Sep. 4, 2015 (now U.S. Pat. No. 11,111,311, which was issued on Sep. 7, 2021), which is a Continuation of U.S. application Ser. No. 13/575,861, filed Jul. 27, 2012 (now U.S. Pat. No. 9,127,071, which was issued on Sep. 8, 2015), which is a National Stage Application filed under § 371 of PCT Application No. PCT/JP2011/000485, filed Jan. 28, 2011, which claims the priority based on Japanese Patent Application No. 2010-019391, filed on Jan. 29, 2010. The contents thereof are incorporated herein by reference.

### INCORPORATION BY REFERENCE OF SEQUENCE LISTING

The content of the electronically submitted sequence listing, file name: Q307289_Sequence_Listing_ST26; size: 95.7 KB; and date of creation: Mar. 12, 2025, filed herewith, is incorporated herein by reference in its entirety.

### TECHNICAL FIELD

The present invention relates to an anticancer agent and a method for diagnosing cancer.

### BACKGROUND ART

Small-cell lung cancer accounts for approximately 20% of all lung cancer incidence. The small-cell lung cancer rapidly progresses and is difficult to be surgically removed because lymph node metastasis or distant metastasis has already occurred at the time of diagnosis in many cases. This cancer exhibits high response rates to an anticancer agent in its early stage. Thus, chemotherapy is considered as the first choice for treating the cancer. The cancer, however, immediately becomes resistant to chemotherapy and recurs, resulting in a 3-year survival rate of 5% or lower. Hence, new therapy has been demanded.

Delta-like 3 (DLL3) is a type I membrane protein belonging to Notch ligand family members. DLL3 is necessary for normal somite formation and patterning. Mutations in DLL3 cause rib defects or spondylolysis in autosomal recessive spondylocostal dysostosis patients [Non Patent Literatures 1 and 2]. DLL1 localizes on cell surface and binds to Notch, whereas DLL3 predominantly localizes in the Golgi apparatus and does not bind to Notch [Non Patent Literatures 3 and 4].

There exist previous studies reporting the amplification of the DLL3 gene on chromosome and increased expression of this gene in pancreatic cancer cell lines [Non Patent Literature 5] and increased DLL3 expression in some glioma cases [Non Patent Literature 6]. However, the number of the DLL3 protein on cell surface has not yet been reported. The expression of 10⁵ or more antigen molecules for unmodified antibodies or antigen molecules of 10⁴ order even for defucosylated antibodies having the enhanced ability to induce antibody-dependent cell-mediated cytotoxicity (ADCC) is required for targeting the antigen molecules on cell surface using such antibodies or for killing cancer cells under the anti-tumor mechanism of the ADCC activity [Non Patent Literature 7]. Thus, it is uncertain whether DLL3 is suitable as a therapeutic target based on antibodies.

Mouse anti-DLL3 monoclonal antibodies (MAB4315, R&D Systems, Inc.) are already commercially available as a research reagent.

### CITATION LIST

### Non Patent Literature

Non Patent Literature 1: Bulman, M. P. et al. (2000) Nat Genet 24, 438-441.
Non Patent Literature 2: Turnpenny, P. D. et al. (2003) J Med Genet 40, 333-339.
Non Patent Literature 3: Geffers, I. et al. (2007) J Cell Biol 178, 465-476.
Non Patent Literature 4: Ladi, E. et al. (2005) J Cell Biol 170, 983-992.
Non Patent Literature 5: Phillips, H. S. (2006) Cancer Cell 9, 157-173.
Non Patent Literature 6: Mulledndore, M. E. (2009) Clin Cancer Res 15, 2291-2301.
Non Patent Literature 7: Kenya Shitara (2009) YAKUGAKU ZASSHI 129, 3-9.

### SUMMARY OF INVENTION

### Technical Problem

An object of the present invention is to provide a novel antibody, an anticancer agent comprising the same, and a method for diagnosing cancer using the same.

### Solution to Problem

The present inventors found that DLL3 mRNA expression was increased in small-cell lung cancer. Its expression was low in all normal tissues except for the fetal brain. The present inventors prepared monoclonal antibodies against the DLL3 protein. The antigen level on cell surface measured based on the capability of binding to the antibody was only less than 10⁴ per expressing cell. Unexpectedly, the present inventors found that an antibody that bound to DLL3 via a characteristic epitope in the vicinity of the C terminus of the extracellular domain stably resided on the cell membrane and had an ADCC-inducing activity. Specifically, the present inventors successively screened for an antibody having an anti-tumor activity. Moreover, the present inventors found that an antibody conjugated with toxin had a cytotoxic activity against DLL3-expressing cells. From these findings, the present inventors found that the anti-DLL3 antibody was useful in the diagnosis, prevention, and treatment of primary or metastatic cancer expressing DLL3. Based on these findings, the present invention has been completed.

The present invention provides an antibody which binds to DLL3 protein. Preferably, the antibody of the present invention has a cytotoxic activity. Particularly preferably, the cytotoxic activity is an antibody-dependent cell-mediated cytotoxic activity (ADCC activity). Also preferably, the antibody of the present invention has an internalization activity. Also preferably, the antibody of the present invention is conjugated with a cytotoxic substance. Also preferably, the antibody of the present invention recognizes a region from amino acids 216 to 492 in human DLL3 having the amino acid sequence as set forth in SEQ ID NO: 1.

In another aspect, the present invention provides an antibody which binds to DLL3 protein described in any of the following:
(1) an antibody comprising a heavy chain variable region comprising heavy chain CDR1 having the amino acid sequence as set forth in SEQ ID NO: 12, heavy chain CDR2 having the amino acid sequence as set forth in SEQ ID NO: 13, and heavy chain CDR3 having the amino acid sequence as set forth in SEQ ID NO: 14;
(2) an antibody comprising a heavy chain variable region comprising heavy chain CDR1 having the amino acid sequence as set forth in SEQ ID NO: 24, heavy chain CDR2 having the amino acid sequence as set forth in SEQ ID NO: 25, and heavy chain CDR3 having the amino acid sequence as set forth in SEQ ID NO: 26;
(3) an antibody comprising a heavy chain variable region comprising heavy chain CDR1 having the amino acid sequence as set forth in SEQ ID NO: 36, heavy chain CDR2 having the amino acid sequence as set forth in SEQ ID NO: 37, and heavy chain CDR3 having the amino acid sequence as set forth in SEQ ID NO: 38;
(4) an antibody comprising a heavy chain variable region comprising heavy chain CDR1 having the amino acid sequence as set forth in SEQ ID NO: 48, heavy chain CDR2 having the amino acid sequence as set forth in SEQ ID NO: 49, and heavy chain CDR3 having the amino acid sequence as set forth in SEQ ID NO: 50;
(5) an antibody comprising a light chain variable region comprising light chain CDR1 having the amino acid sequence as set forth in SEQ ID NO: 18, light chain CDR2 having the amino acid sequence as set forth in SEQ ID NO: 19, and light chain CDR3 having the amino acid sequence as set forth in SEQ ID NO: 20;
(6) an antibody comprising a light chain variable region comprising light chain CDR1 having the amino acid sequence as set forth in SEQ ID NO: 30, light chain CDR2 having the amino acid sequence as set forth in SEQ ID NO: 31, and light chain CDR3 having the amino acid sequence as set forth in SEQ ID NO: 32;
(7) an antibody comprising a light chain variable region comprising light chain CDR1 having the amino acid sequence as set forth in SEQ ID NO: 42, light chain CDR2 having the amino acid sequence as set forth in SEQ ID NO: 43, and light chain CDR3 having the amino acid sequence as set forth in SEQ ID NO: 44;
(8) an antibody comprising a light chain variable region comprising light chain CDR1 having the amino acid sequence as set forth in SEQ ID NO: 54, light chain CDR2 having the amino acid sequence as set forth in SEQ ID NO: 55, and light chain CDR3 having the amino acid sequence as set forth in SEQ ID NO: 56;
(9) an antibody comprising a heavy chain variable region comprising heavy chain CDR1 having the amino acid sequence as set forth in SEQ ID NO: 12, heavy chain CDR2 having the amino acid sequence as set forth in SEQ ID NO: 13, and heavy chain CDR3 having the amino acid sequence as set forth in SEQ ID NO: 14, and a light chain variable region comprising light chain CDR1 having the amino acid sequence as set forth in SEQ ID NO: 18, light chain CDR2 having the amino acid sequence as set forth in SEQ ID NO: 19, and light chain CDR3 having the amino acid sequence as set forth in SEQ ID NO: 20;
(10) an antibody comprising a heavy chain variable region comprising heavy chain CDR1 having the amino acid sequence as set forth in SEQ ID NO: 24, heavy chain CDR2 having the amino acid sequence as set forth in SEQ ID NO: 25, and heavy chain CDR3 having the amino acid sequence as set forth in SEQ ID NO: 26, and a light chain variable region comprising light chain CDR1 having the amino acid sequence as set forth in SEQ ID NO: 30, light chain CDR2 having the amino acid sequence as set forth in SEQ ID NO: 31, and light chain CDR3 having the amino acid sequence as set forth in SEQ ID NO: 32;
(11) an antibody comprising a heavy chain variable region comprising heavy chain CDR1 having the amino acid sequence as set forth in SEQ ID NO: 36, heavy chain CDR2 having the amino acid sequence as set forth in SEQ ID NO: 37, and heavy chain CDR3 having the amino acid sequence as set forth in SEQ ID NO: 38, and a light chain variable region comprising light chain CDR1 having the amino acid sequence as set forth in SEQ ID NO: 42, light chain CDR2 having the amino acid sequence as set forth in SEQ ID NO: 43, and light chain CDR3 having the amino acid sequence as set forth in SEQ ID NO: 44;
(12) an antibody comprising a heavy chain variable region comprising heavy chain CDR1 having the amino acid sequence as set forth in SEQ ID NO: 48, heavy chain CDR2 having the amino acid sequence as set forth in SEQ ID NO: 49, and heavy chain CDR3 having the amino acid sequence as set forth in SEQ ID NO: 50, and a light chain variable region comprising light chain CDR1 having the amino acid sequence as set forth in SEQ ID NO: 54, light chain CDR2 having the amino acid sequence as set forth in SEQ ID NO: 55, and light chain CDR3 having the amino acid sequence as set forth in SEQ ID NO: 56;
(13) an antibody comprising one or more amino acid substitutions, deletions, additions, and/or insertions in the antibody of any one of (1) to (12) and has an activity equivalent to that of any of the antibodies (1) to (12); and
(14) an antibody which binds to the same epitope as that in DLL3 protein to which any of the antibodies (1) to (12) bind.

In a further alternative aspect, the present invention provides a pharmaceutical composition comprising any of the antibodies described above as an active ingredient. Preferably, the pharmaceutical composition of the present invention is an anticancer agent. Particularly preferably, the anticancer agent targets lung cancer.

In a further alternative aspect, the present invention provides a method for diagnosing cancer, comprising the following steps:
(a) providing a sample isolated from a test subject; and
(b) detecting the expression level of DLL3 protein or DLL3 gene in the sample. Preferably, the diagnosis method of the present invention is intended for the diagnosis of lung cancer. The present invention also provides a diagnostic agent for cancer comprising any of the antibodies described above.

### BRIEF DESCRIPTION OF DRAWINGS

FIG. 1 shows increased DLL3 expression in small-cell lung cancer and the fetal brain.

FIG. 2 shows the binding of an anti-DLL3 monoclonal antibody to a soluble DLL3 protein.

FIG. 3 shows the binding of an antibody to the DLL3 protein on a cell membrane and the turnover of the cell-bound antibody.

FIG. 4 shows the induction of ADCC by an anti-DLL3 antibody (concentration: 2.5 μg/ml).

FIG. 5 shows the antibody concentration dependence of an ADCC activity against target cells DLL3/BaF3 (a) and NCI-H1184 (b).

FIG. 6 shows the inhibition of cell growth by the uptake of an anti-DLL3 antibody and an anti-mouse secondary antibody Mab-ZAP labeled with toxin.

FIG. 7 shows the binding of a recombinant anti-DLL3 antibody to the DLL3 protein on cell surface.

FIG. 8 shows the binding of a recombinant anti-DLL3 human chimeric antibody to a soluble DLL3 protein and the binding competition of an anti-DLL3 chimeric antibody with a mouse antibody against a soluble DLL3 protein.

FIG. 9 shows the schematic structure of full-length and soluble DLL3 proteins and a site recognized by an anti-DLL3 antibody.

FIG. 10 shows the binding of a recombinant anti-DLL3 human chimeric antibody DL306 to the DLL3 protein on cell surface.

FIG. 11 shows the antibody concentration dependence of the ADCC activity of an anti-DLL3 human chimeric antibody against target cells DLL3/BaF3 and NCI-H1184.

FIG. 12 shows the antibody concentration dependence of the ADCC activity of an anti-DLL3 mouse low-fucose antibody against target cells DLL3/BaF3 and NCI-H1184.

### DESCRIPTION OF EMBODIMENTS

### DLL3

The amino acid sequence of DLL3 (Delta-like 3) is known in the art. For example, the amino acid sequence of human DLL3 is as set forth in SEQ ID NO: 1 (NM 016941), and the amino acid sequence of mouse DLL3 is as set forth in SEQ ID NO: 2 (NM_007866).

The DLL3 protein used in the present invention may be a DLL3 protein having the sequence described above or may be a modified protein having a sequence derived from the sequence described above by the modification of one or more amino acids. Examples of the modified protein having a sequence derived from the sequence described above by the modification of one or more amino acids can include polypeptides having 70% or more, preferably 80% or more, more preferably 90% or more, even more preferably 95% or more homology to the amino acid sequence. Alternatively, partial peptides of these DLL3 proteins may be used.

The DLL3 protein used in the present invention is not limited by its origin and is preferably a human DLL3 protein.

### Anti-DLL3 Antibody

The anti-DLL3 antibody used in the present invention needs only to bind to the DLL3 protein and is not particularly limited by its origin, type, shape, etc. Specifically, an antibody can be used, such as a non-human animal-derived antibody (e.g., a mouse, rat, or camel antibody), a human-derived antibody, a chimeric antibody, or a humanized antibody. The anti-DLL3 antibody used in the present invention may be a polyclonal or monoclonal antibody and is preferably a monoclonal antibody.

The anti-DLL3 antibody used in the present invention is preferably an anti-human DLL3 antibody. The anti-human DLL3 antibody may be an antibody which specifically binds to human DLL3 or may be an antibody which binds to human DLL3 as well as non-human animal-derived DLL3 (e.g., mouse DLL3).

The anti-DLL3 antibody used in the present invention can be obtained as a polyclonal or monoclonal antibody using means known in the art. The anti-DLL3 antibody used in the present invention is particularly preferably a mammal-derived monoclonal antibody. The mammal-derived monoclonal antibody encompasses, for example, those produced by hybridomas and those produced by hosts transformed with expression vectors containing an antibody gene by a genetic engineering approach.

The anti-DLL3 antibody of the present invention may be modified with various molecules such as polyethylene glycol (PEG). As described later, the anti-DLL3 antibody of the present invention may also be modified with a chemotherapeutic agent, a radioactive chemical, or the like, having a cytotoxic activity.

Examples of the antibody used in the present invention, which recognizes DLL3 and binds thereto, can include the following antibodies:
(1) an antibody (DL301) comprising a heavy chain variable region comprising heavy chain CDR1 having the amino acid sequence as set forth in SEQ ID NO: 12, heavy chain CDR2 having the amino acid sequence as set forth in SEQ ID NO: 13, and heavy chain CDR3 having the amino acid sequence as set forth in SEQ ID NO: 14;
(2) an antibody (DL306) comprising a heavy chain variable region comprising heavy chain CDR1 having the amino acid sequence as set forth in SEQ ID NO: 24, heavy chain CDR2 having the amino acid sequence as set forth in SEQ ID NO: 25, and heavy chain CDR3 having the amino acid sequence as set forth in SEQ ID NO: 26;
(3) an antibody (DL309) comprising a heavy chain variable region comprising heavy chain CDR1 having the amino acid sequence as set forth in SEQ ID NO: 36, heavy chain CDR2 having the amino acid sequence as set forth in SEQ ID NO: 37, and heavy chain CDR3 having the amino acid sequence as set forth in SEQ ID NO: 38;
(4) an antibody (DL312) comprising a heavy chain variable region comprising heavy chain CDR1 having the amino acid sequence as set forth in SEQ ID NO: 48, heavy chain CDR2 having the amino acid sequence as set forth in SEQ ID NO: 49, and heavy chain CDR3 having the amino acid sequence as set forth in SEQ ID NO: 50;
(5) an antibody (DL301) comprising a light chain variable region comprising light chain CDR1 having the amino acid sequence as set forth in SEQ ID NO: 18, light chain CDR2 having the amino acid sequence as set forth in SEQ ID NO: 19, and light chain CDR3 having the amino acid sequence as set forth in SEQ ID NO: 20;
(6) an antibody (DL306) comprising a light chain variable region comprising light chain CDR1 having the amino acid sequence as set forth in SEQ ID NO: 30, light chain CDR2 having the amino acid sequence as set forth in SEQ ID NO: 31, and light chain CDR3 having the amino acid sequence as set forth in SEQ ID NO: 32;
(7) an antibody (DL309) comprising a light chain variable region comprising light chain CDR1 having the amino acid sequence as set forth in SEQ ID NO: 42, light chain CDR2 having the amino acid sequence as set forth in SEQ ID NO: 43, and light chain CDR3 having the amino acid sequence as set forth in SEQ ID NO: 44;
(8) an antibody (DL312) comprising a light chain variable region comprising light chain CDR1 having the amino acid sequence as set forth in SEQ ID NO: 54, light chain CDR2 having the amino acid sequence as set forth in SEQ ID NO: 55, and light chain CDR3 having the amino acid sequence as set forth in SEQ ID NO: 56;
(9) an antibody (DL301) comprising a heavy chain variable region comprising heavy chain CDR1 having the amino acid sequence as set forth in SEQ ID NO: 12, heavy chain CDR2 having the amino acid sequence as set forth in SEQ ID NO: 13, and heavy chain CDR3 having the amino acid sequence as set forth in SEQ ID NO: 14, and a light chain variable region comprising light chain CDR1 having the amino acid sequence as set forth in SEQ ID NO: 18, light chain CDR2 having the amino acid sequence as set forth in SEQ ID NO: 19, and light chain CDR3 having the amino acid sequence as set forth in SEQ ID NO: 20;
(10) an antibody (DL306) comprising a heavy chain variable region comprising heavy chain CDR1 having the amino acid sequence as set forth in SEQ ID NO: 24, heavy chain CDR2 having the amino acid sequence as set forth in SEQ ID NO: 25, and heavy chain CDR3 having the amino acid sequence as set forth in SEQ ID NO: 26, and a light chain variable region comprising light chain CDR1 having the amino acid sequence as set forth in SEQ ID NO: 30, light chain CDR2 having the amino acid sequence as set forth in SEQ ID NO: 31, and light chain CDR3 having the amino acid sequence as set forth in SEQ ID NO: 32;
(11) an antibody (DL309) comprising a heavy chain variable region comprising heavy chain CDR1 having the amino acid sequence as set forth in SEQ ID NO: 36, heavy chain CDR2 having the amino acid sequence as set forth in SEQ ID NO: 37, and heavy chain CDR3 having the amino acid sequence as set forth in SEQ ID NO: 38, and a light chain variable region comprising light chain CDR1 having the amino acid sequence as set forth in SEQ ID NO: 42, light chain CDR2 having the amino acid sequence as set forth in SEQ ID NO: 43, and light chain CDR3 having the amino acid sequence as set forth in SEQ ID NO: 44;
(12) an antibody (DL312) comprising a heavy chain variable region comprising heavy chain CDR1 having the amino acid sequence as set forth in SEQ ID NO: 48, heavy chain CDR2 having the amino acid sequence as set forth in SEQ ID NO: 49, and heavy chain CDR3 having the amino acid sequence as set forth in SEQ ID NO: 50, and a light chain variable region comprising light chain CDR1 having the amino acid sequence as set forth in SEQ ID NO: 54, light chain CDR2 having the amino acid sequence as set forth in SEQ ID NO: 55, and light chain CDR3 having the amino acid sequence as set forth in SEQ ID NO: 56;
(13) an antibody that is derived from any of the antibodies (1) to (12) by the substitution, deletion, addition, and/or insertion of one or more amino acids and has an activity equivalent to that of any of the antibodies (1) to (12); and
(14) an antibody which binds to the same epitope as that in DLL3 protein to which any of the antibodies (1) to (12) bind.

The antibodies (1) to (12) may contain constant regions. The constant regions used are not particularly limited, and any constant region may be used. Preferable examples of the constant regions used in the present invention can include human-derived constant regions. For example, a human IgG1-derived, human IgG2-derived, human IgG3-derived, or human IgG4-derived constant region can be used as a heavy chain constant region. Also, for example, human κ chain-derived or human λ chain-derived constant region can be used as a light chain constant region. The constant regions used in the present invention may be constant regions having a native sequence or may be modified constant regions having a sequence derived from the native sequence by the modification of one or more amino acids.

The antibodies (1) to (12) may contain FRs. The FRs used are not particularly limited, and any FR may be used as long as the resulting antibody maintains its binding activity against human DLL3. Preferable examples of the FRs used in the present invention can include human antibody-derived FRs. Since the technique of FR replacement with the antigen binding activity of an antibody maintained is known in the art, those skilled in the art can appropriately select FRs. The FRs used in the present invention may be FRs having a native sequence or may be FRs having a sequence derived from the native sequence by the modification of one or more amino acids.

Examples of the heavy chain variable region comprising heavy chain CDR1 having the amino acid sequence as set forth in SEQ ID NO: 12, heavy chain CDR2 having the amino acid sequence as set forth in SEQ ID NO: 13, and heavy chain CDR3 having the amino acid sequence as set forth in SEQ ID NO: 14 can include a heavy chain variable region having the amino acid sequence as set forth in SEQ ID NO: 9. Also, examples of the heavy chain comprising the heavy chain variable region can include a heavy chain having the amino acid sequence of SEQ ID NO: 10 or 11.

Examples of the heavy chain variable region comprising heavy chain CDR1 having the amino acid sequence as set forth in SEQ ID NO: 24, heavy chain CDR2 having the amino acid sequence as set forth in SEQ ID NO: 25, and heavy chain CDR3 having the amino acid sequence as set forth in SEQ ID NO: 26 can include a heavy chain variable region having the amino acid sequence as set forth in SEQ ID NO: 21. Also, examples of the heavy chain comprising the heavy chain variable region can include a heavy chain having the amino acid sequence of SEQ ID NO: 22 or 23.

Examples of the heavy chain variable region comprising heavy chain CDR1 having the amino acid sequence as set forth in SEQ ID NO: 36, heavy chain CDR2 having the amino acid sequence as set forth in SEQ ID NO: 37, and heavy chain CDR3 having the amino acid sequence as set forth in SEQ ID NO: 38 can include a heavy chain variable region having the amino acid sequence as set forth in SEQ ID NO: 33. Also, examples of the heavy chain comprising the heavy chain variable region can include a heavy chain having the amino acid sequence of SEQ ID NO: 34 or 35.

Examples of the heavy chain variable region comprising heavy chain CDR1 having the amino acid sequence as set forth in SEQ ID NO: 48, heavy chain CDR2 having the amino acid sequence as set forth in SEQ ID NO: 49, and heavy chain CDR3 having the amino acid sequence as set forth in SEQ ID NO: 50 can include a heavy chain variable region having the amino acid sequence as set forth in SEQ ID NO: 45. Also, examples of the heavy chain comprising the heavy chain variable region can include a heavy chain having the amino acid sequence of SEQ ID NO: 46 or 47.

Examples of the light chain variable region comprising light chain CDR1 having the amino acid sequence as set forth in SEQ ID NO: 18, light chain CDR2 having the amino acid sequence as set forth in SEQ ID NO: 19, and light chain CDR3 having the amino acid sequence as set forth in SEQ ID NO: 20 can include a light chain variable region having the amino acid sequence as set forth in SEQ ID NO: 15. Also, examples of the light chain comprising the light chain variable region can include a light chain having the amino acid sequence of SEQ ID NO: 16 or 17.

Examples of the light chain variable region comprising light chain CDR1 having the amino acid sequence as set forth in SEQ ID NO: 30, light chain CDR2 having the amino acid sequence as set forth in SEQ ID NO: 31, and light chain CDR3 having the amino acid sequence as set forth in SEQ ID NO: 32 can include a light chain variable region having the amino acid sequence as set forth in SEQ ID NO: 27. Also, examples of the light chain comprising the light chain variable region can include a light chain having the amino acid sequence of SEQ ID NO: 28 or 29.

Examples of the light chain variable region comprising light chain CDR1 having the amino acid sequence as set forth in SEQ ID NO: 42, light chain CDR2 having the amino acid sequence as set forth in SEQ ID NO: 43, and light chain CDR3 having the amino acid sequence as set forth in SEQ ID NO: 44 can include a light chain variable region having the amino acid sequence as set forth in SEQ ID NO: 39. Also, examples of the light chain comprising the light chain variable region can include a light chain having the amino acid sequence of SEQ ID NO: 40 or 41.

Examples of the light chain variable region comprising light chain CDR1 having the amino acid sequence as set forth in SEQ ID NO: 54, light chain CDR2 having the amino acid sequence as set forth in SEQ ID NO: 55, and light chain CDR3 having the amino acid sequence as set forth in SEQ ID NO: 56 can include a light chain variable region having the amino acid sequence as set forth in SEQ ID NO: 51. Also, examples of the light chain comprising the light chain variable region can include a light chain having the amino acid sequence of SEQ ID NO: 52 or 53.

In the present invention, the phrase “having an activity equivalent to that of the antibody of the present invention” refers to having a DLL3 binding activity, an internalization activity, and/or a cytotoxic activity (ADCC activity, etc.) against DLL3-expressing cells equivalent thereto. In the present invention, the equivalent activity is not necessarily required to be an identical activity and may be, for example, 50% or more, preferably 70% or more, more preferably 90% or more activity compared with the activity of any of the antibodies (1) to (12). Examples of the upper limit of the activity can include, but not particularly limited to, 1000% or less, 500% or less, 300% or less, 150% or less, and 100% or less.

An antibody derived from the antibody of the present invention by the substitution, deletion, addition, and/or insertion of one or more amino acids is also incorporated in the scope of the present invention and may be prepared artificially or occur naturally. Examples of a method for introducing a mutation in the polypeptide include site-directed mutagenesis (Hashimoto-Gotoh, T. et al. (1995) Gene 152, 271-275, Zoller, M J, and Smith, M. (1983) Methods Enzymol. 100, 468-500, Kramer, W. et al. (1984) Nucleic Acids Res. 12, 9441-9456, Kramer W, and Fritz HJ (1987) Methods. Enzymol. 154, 350-367, Kunkel, TA (1985) Proc Natl Acad Sci USA. 82, 488-492, Kunkel (1988) Methods Enzymol. 85, 2763-2766). This is one of methods well known by those skilled in the art for preparing a polypeptide functionally equivalent to a certain polypeptide. Those skilled in the art can appropriately introduce a mutation in the antibody of the present invention using such a method and thereby prepare an antibody functionally equivalent to the antibody. Moreover, amino acid mutations may occur in the natural world. Such an antibody that has an amino acid sequence derived from the amino acid sequence of the antibody of the present invention by the mutation of one or more amino acids and is functionally equivalent to the antibody is also encompassed by the antibody of the present invention.

The number of amino acids mutated in such a variant is usually within 50 amino acids, preferably within 30 amino acids, more preferably within 10 amino acids (e.g., within 5 amino acids).

For amino acid residues to be mutated, it is preferred that this mutation should be performed conservatively between amino acids having the same side chain property. For example, the following classification based on the properties of amino acid side chains has been established: hydrophobic amino acids (A, I, L, M, F, P, W, Y, and V), hydrophilic amino acids (R, D, N, C, E, Q, G, H, K, S, and T),
amino acids having an aliphatic side chain (G, A, V, L, I, and P),
amino acids having a hydroxy group-containing side chain (S, T, and Y),
amino acids having a sulfur atom-containing side chain (C and M),
amino acids having a side chain containing carboxylic acid and amide (D, N, E, and Q),
amino acids having a base-containing side chain (R, K, and H), and
amino acids having an aromatic group-containing side chain (H, F, Y, and W)
(all symbols within the parentheses represent single letter codes of amino acids).

A polypeptide having an amino acid sequence modified from a certain amino acid sequence by the deletion and/or addition of one or more amino acid residue(s) and/or the substitution thereof by other amino acids is already known to maintain the biological activity of the original polypeptide (Mark, D. F. et al., Proc. Natl. Acad. Sci. USA (1984) 81, 5662-5666, Zoller, M. J. and Smith, M., Nucleic Acids Research (1982) 10, 6487-6500, Wang, A. et al., Science 224, 1431-1433, Dalbadie-McFarland, G. et al., Proc. Natl. Acad. Sci. USA (1982) 79, 6409-6413). Specifically, when amino acids in an amino acid sequence constituting a certain polypeptide are substituted by amino acids classified in the same group thereas, it is generally said that the polypeptide is likely to maintain its activity. In the present invention, the substitution between amino acids within the same amino acid group described above is referred to as conservative substitution.

The present invention also provides an antibody which binds to the same epitope as that to which any of the antibodies (1) to (12) bind. Specific examples of the antibodies (1) to (12) can include antibodies DL301, DL306, DL309, and DL312 described below in Examples. Specifically, the present invention also provides an antibody recognizing the same epitope as that recognized by any of these antibodies.

Whether or not an analyte antibody shares an epitope with a certain antibody can be confirmed based on their competition for the same epitope. The competition between the antibodies is detected by cross-blocking assay or the like. The cross-blocking assay is preferably, for example, competitive ELISA assay.

Specifically, the cross-blocking assay involves preincubating DLL3 proteins coated on the wells of a microtiter plate in the presence or absence of a candidate competing antibody and then adding thereto the anti-DLL3 antibody of the present invention. The amount of the anti-DLL3 antibody of the present invention bound to the DLL3 protein in each well indirectly correlates with the binding capability of the candidate competing antibody (analyte antibody) that competes therewith for binding to the same epitope. Specifically, the higher affinity of the analyte antibody for the same epitope results in the smaller amount of the anti-DLL3 antibody of the present invention bound to the DLL3 protein-coated well and instead, the larger amount of the analyte antibody bound to the DLL3 protein-coated well.

The amount of each antibody bound to the well can be determined easily by labeling the antibody in advance. For example, a biotinylated antibody can be assayed using an avidin-peroxidase conjugate and an appropriate substrate. The cross-blocking assay using enzyme (e.g., peroxidase) labeling is particularly called competitive ELISA assay. The antibody may be labeled with any of other detectable or measurable labeling materials. Specifically, for example, radiolabeling or fluorescent labeling is known in the art.

Furthermore, when the analyte antibody has constant regions derived from a species different from that of the anti-DLL3 antibody of the present invention, the amount of any antibody bound to the well can also be measured using a labeled antibody that recognizes any constant region. Alternatively, even antibodies differing in class, albeit derived from the same species, can be measured for their respective amounts bound to the well using antibodies that discriminate each class.

Provided that the candidate antibody can block the binding of the anti-DLL3 antibody by at least 20%, preferably at least 30%, more preferably at least 50%, even more preferably at least 80%, compared to the binding activity obtained in the control test performed in the absence of the competing antibody, this candidate antibody is determined as an antibody that binds to substantially the same epitope as that to which the anti-DLL3 antibody of the present invention binds or as an antibody that competes therewith for the binding to the same epitope.

Further examples of the antibody of the present invention can include an antibody recognizing a region from amino acids 27 to 175 in human DLL3 (SEQ ID NO: 1) and an antibody recognizing a region from amino acids 216 to 492 in human DLL3. Examples of another aspect of the antibody of the present invention can include an antibody that binds to human DLL3 but does not bind to a polypeptide (DLL3delta1-Fc) consisting of the amino acid sequence of SEQ ID NO: 6 or a polypeptide (DLL3delta2-Fc) consisting of the amino acid sequence of SEQ ID NO: 7, and an antibody that binds to human DLL3 and also binds to the polypeptide (DLL3delta1-Fc) consisting of the amino acid sequence of SEQ ID NO: 6 and the polypeptide (DLL3delta2-Fc) consisting of the amino acid sequence of SEQ ID NO: 7.

The antibody of the present invention has activities such as an ADCC activity and an internalization activity and as such, is useful as a pharmaceutical drug, particularly, an anticancer agent.

### Genetically Recombinant Anti-DLL3 Antibody

The antibody to be administered to humans can be converted to a genetically recombinant antibody that has been engineered artificially, for example, for the purpose of reducing heteroantigenicity in humans. The genetically recombinant antibody encompasses, for example, chimeric antibodies and humanized antibodies. These engineered antibodies can be produced using a method known in the art.

### (1) Chimeric Antibody

The chimeric antibodies refer to antibodies comprising variable and constant regions of different origins ligated with each other. For example, mouse-human heterogeneous chimeric antibodies are antibodies comprising the heavy and light chain variable regions of a mouse antibody and the heavy and light chain constant regions of a human antibody. Mouse antibody variable region-encoding DNAs are ligated with human antibody constant region-encoding DNAs, and the ligation products can be incorporated into expression vectors to prepare chimeric antibody-expressing recombinant vectors. Cells transformed with these vectors (recombinant cells) can be cultured for the expression of the DNA insert to obtain the chimeric antibodies produced during the culture.

Human antibody constant regions are used as the constant regions of the chimeric antibodies. For example, Cγ1, Cγ2, Cγ3, Cγ4, Cμ, Cδ, Cα1, Cα2, and Cε can be used as heavy chain constant regions. Moreover, Cκ and Cλ can be used as light chain constant regions. The amino acid sequences of these constant regions and nucleotide sequences encoding them are known in the art. Moreover, one or more amino acids in the human antibody constant regions can be substituted, deleted, added, and/or inserted for improving the stability of the antibody itself or of its production.

### (2) Humanized Antibody

In general, the chimeric antibodies comprise non-human animal-derived antibody variable regions and human antibody-derived constant regions. By contrast, the humanized antibodies comprise non-human animal-derived antibody complementarity-determining regions (CDRs), human antibody-derived framework regions (FRs), and human antibody-derived constant regions. The humanized antibodies are also called reshaped human antibodies. Specifically, for example, humanized antibodies comprising non-human animal (e.g., mouse) antibody CDRs grafted in human antibodies are known in the art. The humanized antibodies are useful as active ingredients for a therapeutic agent of the present invention, owing to their reduced antigenicity in the human body.

Each antibody variable region usually comprises 3 CDRs flanked by 4 FRs. The CDR regions substantially determine the binding specificity of the antibody. The CDRs have diverse amino acid sequences. On the other hand, amino acid sequences constituting the FRs often exhibit high homology among antibodies having different binding specificities. Therefore, in general, the binding specificity of a certain antibody can allegedly be transplanted to other antibodies through CDR grafting.

General gene recombination approaches are also known for obtaining the humanized antibodies. Specifically, for example, Overlap Extension PCR is known in the art as a method for grafting mouse antibody CDRs into human FRs. The Overlap Extension PCR employs primers for human antibody FR synthesis comprising an additional nucleotide sequence encoding each mouse antibody CDR to be grafted. The primers are prepared for each of the 4 FRs. In the mouse CDR grafting into the human FRs, in general, it is allegedly advantageous to select human FRs highly homologous to mouse FRs for maintaining the CDR functions. Specifically, it is generally preferred to use human FRs comprising amino acid sequences highly homologous to those of the FRs adjacent to the mouse CDRs to be grafted.

Moreover, the nucleotide sequences to be ligated are designed such that they are connected in frame. The human FR-encoding nucleotide sequences are individually synthesized using their respective primers. As a result, products are obtained, which comprise the mouse CDR-encoding DNA added to each FR-encoding sequence. The mouse CDR-encoding nucleotide sequence in each product is designed such that the nucleotide sequence overlaps with another. Subsequently, the overlapping CDR portions are annealed to each other for complementary strand synthesis reaction. Through this reaction, the human FR sequences are ligated via the mouse CDR sequences.

Finally, the full-length gene of the variable region comprising 3 CDRs and 4 FRs ligated is amplified with primers that respectively anneal to the 5′ and 3′ ends thereof and comprise an additional recognition sequence for an appropriate restriction enzyme. The DNA thus obtained and human antibody constant region-encoding DNA can be inserted into expression vectors such that they are fused in frame to prepare vectors for humanized antibody expression. Hosts are transformed with these vectors to establish recombinant cells, which are then cultured for the expression of the humanized antibody-encoding DNA to produce the humanized antibodies into the cultures of the cultured cells (see European Patent Publication No. EP 239400 and International Publication No. WO 96/02576).

The humanized antibodies thus prepared can be evaluated for their binding activities for the antigen by qualitative or quantitative assay. As a result, human antibody FRs can be selected preferably such that they allow CDRs to form a favorable antigen-binding site when ligated via the CDRs. If necessary, FR amino acid residue(s) may be substituted such that the CDRs of the humanized antibody form an appropriate antigen-binding site. For example, a mutation can be introduced in the amino acid sequence of FR by applying the PCR method used in the mouse CDR grafting into the human FRs. Specifically, a mutation of a partial nucleotide sequence can be introduced in the primers annealing to the FR nucleotide sequence. The FR nucleotide sequence synthesized using such primers contains the mutation thus introduced. The variant antibodies having the substituted amino acid(s) can be evaluated for their binding activities for the antigen by the same assay as above to select variant FR sequences having the desired property (Sato, K. et al., Cancer Res, 1993, 53, 851-856).

### (3) Polyvalent Antibody

The antibody of the present invention encompasses not only bivalent antibodies typified by IgG (IgG1, IgG2, IgG4, etc.) but also monovalent antibodies or polyvalent antibodies typified by IgM as long as these antibodies bind to the DLL3 protein. The polyvalent antibody of the present invention encompasses polyvalent antibodies having antigen-binding sites, all of which are the same as each other or some or all of which are different from each other.

### (4) Low-Molecular Antibody

The antibody of the present invention is not limited to whole antibody molecules and may be a low-molecular antibody or a modified form thereof as long as the antibody binds to the DLL3 protein.

The low-molecular antibody encompasses an antibody fragment deficient in a portion of the whole antibody (e.g., whole IgG). Such partial deficiency of the antibody molecule is accepted as long as the resultant antibody fragment is capable of binding to the DLL3 antigen. It is preferred that the antibody fragment according to the present invention should contain one or both of heavy chain variable (VH) and light chain variable (VL) regions. It is also preferred that the antibody fragment according to the present invention should contain CDRs. The number of CDRs contained in the antibody fragment of the present invention is not particularly limited and is preferably at least 6 CDRs: heavy chain CDR1, CDR2, and CDR3 and light chain CDR1, CDR2, and CDR3.

The amino acid sequence of VH or VL can contain substitution, deletion, addition, and/or insertion. Furthermore, the antibody fragment of the present invention may be deficient in a portion of one or both of VH and VL as long as the resultant antibody fragment is capable of binding to the DLL3 antigen. Moreover, its variable region may be chimerized or humanized. Specific examples of the antibody fragment can include Fab, Fab′, F(ab′)2, and Fv. Moreover, specific examples of the low-molecular antibody can include Fab, Fab′, F(ab′)2, Fv, scFv (single chain Fv), Diabody, sc(Fv)2 (single chain (Fv)2), and scFv-Fc. In the present invention, the low-molecular antibody is preferably Diabody or sc(Fv)2. These antibody multimers (e.g., dimmers, trimers, tetramers, and polymers) are also encompassed by the low-molecular antibody of the present invention.

Such fragments of the antibody can be obtained by enzymatically treating the antibody to form antibody fragments. The digestive enzymes cleave the antibody fragment at a particular position to give antibody fragments having a particular structure. For example, papain, pepsin, or plasmin is known in the art as the enzyme for forming the antibody fragments. The papain digestion gives F(ab)2 or Fab, while the pepsin digestion gives F(ab′)2 or Fab′. Alternatively, genes encoding these antibody fragments are constructed, and these genes can be introduced into expression vectors and then expressed in appropriate host cells (see e.g., Co, M. S. et al., J. Immunol. (1994) 152, 2968-2976, Better, M. & Horwitz, A. H. Methods in Enzymology (1989) 178, 476-496, Plueckthun, A. & Skerra, A. Methods in Enzymology (1989) 178, 497-515, Lamoyi, E., Methods in Enzymology (1986) 121, 652-663, Rousseaux, J. et al., Methods in Enzymology (1986) 121, 663-669, Bird, R. E. et al., TIBTECH (1991) 9, 132-137).

The use of a genetic engineering approach for the enzymatically obtained antibody fragments can delete an arbitrary portion of the antibody. The low-molecular antibody according to the present invention may lack an arbitrary region as long as the resulting antibody fragment has binding affinity for DLL3.

### i) Diabody

The Diabody refers to a bivalent antibody fragment constructed by gene fusion (e.g., Holliger P et al., Proc. Natl. Acad. Sci. USA 90: 6444-6448 (1993), EP404,097, and WO 93/11161). The Diabody is a dimer comprising two polypeptide chains. Usually, each of the polypeptide chains constituting the dimer comprises heavy and light chain variable regions linked via a linker on the same chain. The linker in the Diabody is generally too short to allow paring between heavy and light chain variable regions on the same chain. Specifically, the number of amino acid residues constituting the linker is, for example, approximately 5 residues. Therefore, heavy and light chain variable regions encoded on the same polypeptide chain cannot together form a single chain variable region fragment. Instead, they form a dimer by pairing with another single chain variable region fragment. As a result, the Diabody has two antigen-binding sites.

### II) Scfv

The scFv is obtained by linking heavy and light chain variable regions of the antibody. In the scFv, the heavy and light chain variable regions are linked via a linker, preferably, a peptide linker (Huston, J. S. et al., Proc. Natl. Acad. Sci. U.S.A, 1988, 85, 5879-5883). The heavy and light chain variable regions in the scFv can be derived from any of the antibodies described in the present specification. The peptide linker that links the variable regions is not particularly limited. For example, an arbitrary single chain peptide of approximately 3 to 25 residues can be used as the linker. Specifically, for example, a peptide linker described later can be used.

The variable regions of both the chains can be linked, for example, by PCR. First, of DNA sequences encoding the heavy chain or heavy chain variable region of the antibody and DNA sequences encoding the light chain or light chain variable region of the antibody, DNAs encoding the whole or desired partial amino acid sequence are used as templates for linking the variable regions by PCR.

The heavy chain variable region-encoding DNA and the light chain variable region-encoding DNA are separately amplified by PCR using a pair of primers having sequences corresponding to both terminal sequences of each DNA to be amplified. Subsequently, DNA encoding the peptide linker portion is prepared. The DNA encoding the peptide linker can also be synthesized using PCR. Nucleotide sequences that can be linked to the amplification product of each variable region separately synthesized are respectively added to the 5′ sequences of primers used in this PCR. Subsequently, PCR reaction is performed using each DNA of [heavy chain variable region DNA]-[peptide linker DNA]-[light chain variable region DNA] and primers for assembly PCR.

The primers for assembly PCR comprise the combination of a primer annealing to the 5′ sequence of the [heavy chain variable region DNA] and a primer annealing to the 3′ sequence of the [light chain variable region DNA]. Specifically, the primers for assembly PCR are a primer set that is capable of amplifying DNA encoding the full-length sequence of the scFv to be synthesized. By contrast, the [peptide linker DNA] contains an additional nucleotide sequence that can be linked to each variable region DNA. As a result, these DNAs are linked and, further, finally prepared into a full-length scFv amplification product using the primers for assembly PCR. Once the scFv-encoding DNA is prepared, expression vectors containing this DNA and cells transformed with the expression vectors (recombinant cells) can be obtained according to a routine method. Moreover, the resultant recombinant cells can be cultured for the expression of the scFv-encoding DNA to obtain the scFv.

iii) Scfv-Fc

The scFv-Fc is a low-molecular antibody comprising an Fc region fused to scFv (Cellular & Molecular Immunology 2006; 3: 439-443). The origin of the scFv used in the scFv-Fc is not particularly limited, and, for example, scFv derived from IgM can be used. Moreover, the origin of the Fc is not particularly limited, and, for example, Fc derived from human IgG (human IgG1, etc.) can be used. Thus, examples of a preferable aspect of the scFv-Fc can include scFv-Fc comprising an IgM antibody scFv fragment linked to human IgG1 CH2 (e.g., Cγ2) and CH3 (e.g., Cγ3) via the hinge region (Hγ) of human IgG1.

### iv) Sc(Fv)2

The sc(Fv)2 is a low-molecular antibody having a single chain comprising two heavy chain variable regions (VHs) and two light chain variable regions (VLs) linked via linkers or the like (Hudson et al., J Immunol. Methods 1999; 231: 177-189). The sc(Fv)2 can be prepared, for example, by linking scFvs via a linker. Three linkers are usually necessary for linking four antibody variable regions.

Moreover, the sc(Fv)2 is preferably an antibody wherein two VHs and two VLs are aligned as VH, VL, VH, and VL (i.e., [VH]-linker-[VL]-linker-[VH]-linker-[VL]) in this order starting at the N-terminus of the single chain polypeptide.

The order of two VHs and two VLs is not particularly limited to the arrangement described above and may be any order of arrangement. Examples thereof can also include the following arrangements:
[VL]-linker-[VH]-linker-[VH]-linker-[VL]
[VH]-linker-[VL]-linker-[VL]-linker-[VH]
[VH]-linker-[VH]-linker-[VL]-linker-[VL]
[VL]-linker-[VL]-linker-[VH]-linker-[VH]
[VL]-linker-[VH]-linker-[VL]-linker-[VH]

For example, an arbitrary peptide linker or synthetic compound linker (e.g., linkers disclosed in the reference Protein Engineering, 9 (3), 299-305, 1996) that can be introduced by genetic engineering can be used as the linker that links the antibody variable regions. A plurality of the same or different linkers may be used. In the present invention, the peptide linker is preferable. The length of the peptide linker is not particularly limited and can be selected appropriately by those skilled in the art according to the purpose. The number of amino acid residues constituting the peptide linker is usually 1 to 100 amino acids, preferably 3 to 50 amino acids, more preferably 5 to 30 amino acids, particularly preferably 12 to 18 amino acids (e.g., 15 amino acids).

The amino acid sequence constituting the peptide linker can be an arbitrary sequence as long as this sequence does not inhibit the binding effect of the scFv. For example, the following amino acid sequences can be used for the peptide linker:

Ser
Gly • Ser
Gly • Gly • Ser
Ser • Gly • Gly
(SEQ ID NO: 61)
Gly • Gly • Gly • Ser
(SEQ ID NO: 62)
Ser • Gly • Gly • Gly
(SEQ ID NO: 63)
Gly • Gly • Gly • Gly • Ser
(SEQ ID NO: 64)
Ser • Gly • Gly • Gly • Gly
(SEQ ID NO: 65)
Gly • Gly • Gly • Gly • Gly • Ser
(SEQ ID NO: 66)
Ser • Gly • Gly • Gly • Gly • Gly
(SEQ ID NO: 67)
Gly • Gly • Gly • Gly • Gly • Gly • Ser
(SEQ ID NO: 68)
Ser • Gly • Gly • Gly • Gly • Gly • Gly
(SEQ ID NO: 63, when n = 1)
(Gly • Gly • Gly • Gly • Ser)n
(SEQ ID NO: 63, when n = 1)
(Ser • Gly • Gly • Gly • Gly)n
[n represents an integer of 1 or more].

The amino acid sequence of the peptide linker can be selected appropriately by those skilled in the art according to the purpose. For example, the integer n that determines the length of the peptide linker is usually 1 to 5, preferably 1 to 3, more preferably 1 or 2.

Accordingly, examples of a particularly preferable aspect of the sc(Fv)2 according to the present invention can include the following sc(Fv)2: [VH]-peptide linker (15 amino acids)-[VL]-peptide linker (15 amino acids)-[VH]-peptide linker (15 amino acids)-[VL].

Alternatively, the variable regions can also be linked using the chemically synthesized linker (chemical cross-linking agent). Cross-linking agents usually used in the cross-link of peptide compounds or the like can be used in the present invention. For example, chemical cross-linking agents as shown below are known in the art. These cross-linking agents are commercially available:
N-hydroxysuccinimide (NHS),
disuccinimidyl suberate (DSS),
bis(sulfosuccinimidyl) suberate (BS3),
dithiobis(succinimidyl propionate) (DSP),
dithiobis(sulfosuccinimidyl propionate) (DTSSP),
ethylene glycol bis(succinimidyl succinate) (EGS),
ethylene glycol bis(sulfosuccinimidyl succinate) (sulfo-EGS),
disuccinimidyl tartrate (DST), disulfosuccinimidyl tartrate (sulfo-DST),
bis[2-(succinimidoxycarbonyloxy)ethyl]sulfone (BSOCOES), and
bis[2-(sulfosuccinimidoxycarbonyloxy)ethyl]sulfone (sulfo-BSOCOES), etc.

### Activity of Anti-DLL3 Antibody

### (1) Cytotoxic Activity

For the treatment of cell-proliferative disease such as cancer, it is preferred that the antibody should maintain its effector activity. Specifically, the preferable antibody according to the present invention has both of a binding affinity for DLL3 and effector functions. The effector functions of the antibody encompass an antibody-dependent cell-mediated cytotoxic (ADCC) activity and a complement-dependent cytotoxic (CDC) activity. The therapeutic antibody according to the present invention particularly preferably possesses an ADCC activity as effector functions.

The antibody of the present invention used for the therapeutic purpose is preferably an antibody having a cytotoxic activity.

Examples of the cytotoxic activity according to the present invention can include ADCC and CDC activities. In the present invention, the ADCC activity means the activity of damaging target cells through the binding of Fcγ receptor-bearing cells (immunocytes, etc.) via the Fcγ receptors to the Fc domains of antibodies specifically attached to the cell surface antigens of the target cells. On the other hand, the CDC activity means a cytotoxic activity mediated by the complement system.

Whether or not the anti-DLL3 antibody has an ADCC activity or has a CDC activity can be determined by a method known in the art (e.g., Current protocols in Immunology, Chapter 7. Immunologic studies in humans, Editor, John E, Coligan et al., John Wiley & Sons, Inc., (1993)). Specifically, effector cells, a complement solution, and target cells are first prepared.

### i) Preparation of Effector Cells

The spleens are excised from CBA/N mice or the like, and spleen cells are separated therefrom in an RPMI1640 medium (manufactured by Invitrogen Corp.). The cells can be washed with this medium containing 10% fetal bovine serum (FBS, manufactured by HyClone Laboratories, Inc.) and then adjusted to a cell concentration of 5×10⁶ cells/ml to prepare effector cells.

### ii) Preparation of Complement Solution

Baby Rabbit Complement (manufactured by CEDARLANE Laboratories Ltd.) can be diluted 10-fold with a medium (manufactured by Invitrogen Corp.) containing 10% FBS to prepare a complement solution.

iii) Preparation of Target Cell

Cells expressing DLL3 proteins can be cultured at 37° C. for 1 hour, together with 0.2 mCi ⁵¹Cr-sodium chromate (manufactured by GE Healthcare Bio-Sciences Corp.), in a DMEM medium containing 10% FBS to radiolabel the target cells. Cells transformed with DLL3 protein-encoding genes, small-cell lung cancer cell lines, or the like can be used as the cells expressing DLL3 proteins. The cells thus radiolabeled can be washed three times with an RPMI1640 medium containing 10% FBS and adjusted to a cell concentration of 2×10⁵ cells/ml to prepare the target cells.

The ADCC or CDC activity can be assayed by a method described below. For the ADCC activity assay, the target cells and the anti-DLL3 antibody (50 μl each) are added to a U-bottom 96-well plate (manufactured by Becton, Dickinson and Company) and reacted for 15 minutes on ice. Then, 100 μl of the effector cells is added to the plate, and the cells are cultured for 4 hours in a CO₂ incubator. The final concentration of the antibody is set to 0 or 10 μg/ml. After the culture, 100 μl of the supernatant is collected, and the radioactivity is measured using a gamma counter (COBRA II AUTO-GAMMA, MODEL D5005, manufactured by Packard Instrument Company). The cytotoxic activity (%) can be calculated based on the calculation formula (A−C)/(B−C)×100 using the obtained value. In the formula, A represents radioactivity (cpm) from each sample; B represents radioactivity (cpm) from a sample supplemented with 1% NP-40 (manufactured by Nacalai Tesque, Inc.); and C represents radioactivity (cpm) from a sample containing only the target cells.

On the other hand, for the CDC activity assay, the target cells and the anti-DLL3 antibody (50 μl each) are added to a flat-bottomed 96-well plate (manufactured by Becton, Dickinson and Company) and reacted for 15 minutes on ice. Then, 100 μl of the complement solution is added to the plate, and the cells are cultured for 4 hours in a CO₂ incubator. The final concentration of the antibody is set to 0 or 3 μg/ml. After the culture, 100 μl of the supernatant is collected, and the radioactivity is measured using a gamma counter. The cytotoxic activity can be calculated in the same way as in the ADCC activity assay.

By contrast, in the cytotoxic activity assay using antibody conjugates, the target cells and the anti-DLL3 antibody conjugates (50 μl each) are added to a flat-bottomed 96-well plate (manufactured by Becton, Dickinson and Company) and reacted for 15 minutes on ice. The cells are cultured for 1 to 4 hours in a CO₂ incubator. The final concentration of the antibody is set to 0 or 3 μg/ml. After the culture, 100 μl of the supernatant is collected, and the radioactivity is measured using a gamma counter. The cytotoxic activity can be calculated in the same way as in the ADCC activity assay.

### (2) Conjugated Antibody

The antibody may be conjugated with a cytotoxic substance such as a chemotherapeutic agent, a toxic peptide, or a radioactive chemical. Such a modified antibody (hereinafter, referred to as an antibody conjugate) can be obtained by chemically modifying the obtained antibody. A method for the antibody modification has already been established in the art.

Examples of the chemotherapeutic agent whose cytotoxic activity functions through the conjugation to the anti-DLL3 antibody can include the following chemotherapeutic agents: azaribine, anastrozole, azacytidine, bleomycin, bortezomib, bryostatin-1, busulfan, camptothecin, 10-hydroxycamptothecin, carmustine, celebrex, chlorambucil, cisplatin, irinotecan, carboplatin, cladribine, cyclophosphamide, cytarabine, dacarbazine, docetaxel, dactinomycin, daunomycin glucuronide, daunorubicin, dexamethasone, diethylstilbestrol, doxorubicin, doxorubicin glucuronide, epirubicin, ethinyl estradiol, estramustine, etoposide, etoposide glucuronide, floxuridine, fludarabine, flutamide, fluorouracil, fluoxymesterone, gemcitabine, hydroxyprogesterone caproate, hydroxyurea, idarubicin, ifosfamide, leucovorin, lomustine, mechlorethamine, medroxyprogesterone acetate, megestrol acetate, melphalan, mercaptopurine, methotrexate, mitoxantrone, mithramycin, mitomycin, mitotane, phenylbutyrate, prednisone, procarbazine, paclitaxel, pentostatin, semustine, streptozocin, tamoxifen, taxanes, taxol, testosterone propionate, thalidomide, thioguanine, thiotepa, teniposide, topotecan, uracil mustard, vinblastine, vinorelbine, vincristine.

The chemotherapeutic agent is preferably a low-molecular chemotherapeutic agent. The low-molecular chemotherapeutic agent is unlikely to interfere with the antibody functions even after its conjugation to the antibody. In the present invention, the low-molecular chemotherapeutic agent usually has a molecular weight of 100 to 2000, preferably 200 to 1000. All of the chemotherapeutic agents exemplified above are low-molecular chemotherapeutic agents. These chemotherapeutic agents according to the present invention encompass prodrugs that are converted in vivo to active chemotherapeutic agents. The prodrug activation may be enzymatic conversion or nonenzymatic conversion.

Moreover, the antibody may be modified with the toxic peptide. Examples of the toxic peptide can include the following: Diphtheria toxin A Chain (Langone J. J., et al., Methods in Enzymology, 93, 307-308, 1983), Pseudomonas Exotoxin (Nature Medicine, 2, 350-353, 1996), Ricin A Chain (Fulton R. J., et al., J. Biol. Chem., 261, 5314-5319, 1986; Sivam G., et al., Cancer Res., 47, 3169-3173, 1987; Cumber A. J. et al., J. Immunol. Methods, 135, 15-24, 1990; Wawrzynczak E. J., et al., Cancer Res., 50, 7519-7562, 1990; Gheeite V., et al., J. Immunol. Methods, 142, 223-230, 1991); Deglicosylated Ricin A Chain (Thorpe P. E., et al., Cancer Res., 47, 5924-5931, 1987); Abrin A Chain (Wawrzynczak E. J., et al., Br. J. Cancer, 66, 361-366, 1992; Wawrzynczak E. J., et al., Cancer Res., 50, 7519-7562, 1990; Sivam G., et al., Cancer Res., 47, 3169-3173, 1987; Thorpe P. E., et al., Cancer Res., 47, 5924-5931, 1987); Gelonin (Sivam G., et al., Cancer Res., 47, 3169-3173, 1987; Cumber A. J. et al., J. Immunol. Methods, 135, 15-24, 1990; WawrzynczakE. J., et al., Cancer Res., 50, 7519-7562, 1990; Bolognesi A., et al., Clin. exp. Immunol., 89, 341-346, 1992); PAP-s; Pokeweed anti-viral protein fromseeds (Bolognesi A., et al., Clin. exp. Immunol., 89, 341-346, 1992); Briodin (Bolognesi A., et al., Clin. exp. Immunol., 89, 341-346, 1992); Saporin (Bolognesi A., et al., Clin. exp. Immunol., 89, 341-346, 1992); Momordin (Cumber A. J., et al., J. Immunol. Methods, 135, 15-24, 1990; Wawrzynczak E. J., et al., Cancer Res., 50, 7519-7562, 1990; Bolognesi A., et al., Clin. exp. Immunol., 89, 341-346, 1992); Momorcochin (Bolognesi A., et al., Clin. exp. Immunol., 89, 341-346, 1992); Dianthin 32 (Bolognesi A., et al., Clin. exp. Immunol., 89, 341-346, 1992); Dianthin 30 (Stirpe F., Barbieri L., FEBS letter 195, 1-8, 1986); Modeccin (Stirpe F., Barbieri L., FEBS letter 195, 1-8, 1986); Viscumin (Stirpe F., Barbieri L., FEBS letter 195, 1-8, 1986); Volkesin (Stirpe F., Barbieri L., FEBS letter 195, 1-8, 1986); Dodecandrin (Stirpe F., Barbieri L., FEBS letter 195, 1-8, 1986); Tritin (Stirpe F., Barbieri L., FEBS letter 195, 1-8, 1986); Luffin (Stirpe F., Barbieri L., FEBS letter 195, 1-8, 1986); Trichokirin (Casellas P., et al., Eur. J. Biochem. 176, 581-588, 1988; Bolognesi A., et al., Clin. exp. Immunol., 89, 341-346, 1992).

In the present invention, the radioactive chemical refers to a chemical containing a radioisotope. The radioisotope is not particularly limited, and any radioisotope may be used. For example, ³²P, ¹⁴C, ¹²⁵I, ³H, ¹³¹I, ¹⁸⁶Re, or ¹⁸⁸Re can be used.

In another aspect, one or two or more low-molecular chemotherapeutic agents and one or two or more toxic peptides can be used in combination in the antibody modification. The anti-DLL3 antibody can be conjugated to the low-molecular chemotherapeutic agent via a covalent or noncovalent bond. A method for preparing such a chemotherapeutic agent-conjugated antibody is known in the art.

A proteinous agent or toxin can be conjugated to the antibody by a genetic engineering approach. Specifically, for example, DNA encoding the toxic peptide and DNA encoding the anti-DLL3 antibody are fused in frame with each other, and this fused DNA can be incorporated into expression vectors to construct recombinant vectors. The vectors are introduced into appropriate host cells, and the resultant transformed cells are cultured. The DNA insert can be expressed by the cells to obtain toxic peptide-conjugated anti-DLL3 antibodies as fusion proteins. For obtaining antibody-fusion proteins, the proteinous agent or toxin is generally located on the C-terminal side of the antibody. A peptide linker may be allowed to intervene between the antibody and the proteinous agent or toxin.

### (3) Bispecific Antibody

Furthermore, the antibody of the present invention may be a bispecific antibody. The bispecific antibody refers to an antibody having, in the same antibody molecule, variable regions that recognize different epitopes. In the present invention, the bispecific antibody can have antigen-binding sites that recognize different epitopes on the DLL3 molecule. Thus, two such bispecific antibody molecules can bind to one DLL3 molecule. As a result, stronger cytotoxic effect can be expected.

Alternatively, the bispecific antibody of the present invention may have antigen-binding sites, one of which recognizes DLL3 and the other of which recognizes a cytotoxic substance. The cytotoxic substance specifically encompasses, for example, a chemotherapeutic agent, a toxic peptide, and a radioactive chemical. Such a bispecific antibody binds to cells expressing DLL3, while it captures the cytotoxic substance. As a result, the cytotoxic substance can be allowed to directly act on the cells expressing DLL3. Specifically, the bispecific antibody that recognizes the cytotoxic substance can specifically damage tumor cells and inhibit the growth of the tumor cells.

Moreover, in the present invention, a bispecific antibody comprising a DLL3-binding site combined with an antigen-binding site that recognizes an antigen other than DLL3 may be used. The antigen-binding site that can be combined therewith in such a bispecific antibody recognizes, for example, an antigen that is specifically expressed on the surface of target cancer cells, as with DLL3, but is different from DLL3.

A method for producing the bispecific antibody is known in the art. For example, two antibodies differing in antigen recognized thereby can be bound to prepare the bispecific antibody. Each of the antibodies bound may be a ½ molecule having heavy and light chains or may be a ¼ molecule consisting of heavy chains. Alternatively, different monoclonal antibody-producing hybridomas may be fused to prepare fusion cells producing bispecific antibodies. Furthermore, the bispecific antibody can be prepared by a genetic engineering approach.

The antigen binding activity of the antibody can be determined using means known in the art (Antibodies A Laboratory Manual. Ed Harlow, David Lane, Cold Spring Harbor Laboratory, 1988). For example, ELISA (enzyme-linked immunosorbent assay), EIA (enzyme immunoassay), RIA (radioimmunoassay), or fluoroimmunoassay can be used.

### (4) Modification of Sugar Chain

The antibody of the present invention may be an antibody having a modified sugar chain. It is known that the cytotoxic activities of antibodies can be enhanced by modifying their sugar chains. For example, glycosylated antibodies (WO 99/54342, etc.), antibodies deficient in fucose added to their sugar chains (WO 00/61739, WO 02/31140, etc.), and antibodies having a sugar chain having bisecting GlcNAc (WO 02/79255, etc.) are known in the art as the antibody having a modified sugar chain.

### (5) Internalization Activity

Moreover, the antibody of the present invention may have an internalization activity. In the present invention, the “antibody having an internalization activity” means an antibody that is transported into cells (cytoplasms, vesicles, other organelles, etc.) through its binding to DLL3.

Whether or not the antibody has an internalization activity can be confirmed by a method generally known by those skilled in the art and can be confirmed by, for example, a method involving contacting labeling material-bound anti-DLL3 antibodies with DLL3-expressing cells and confirming whether or not the labeling material is incorporated into the cells, or a method involving contacting cytotoxic substance-conjugated anti-DLL3 antibodies with DLL3-expressing cells and confirming whether or not the death of the DLL3-expressing cells is induced.

More specifically, the internalization activity of the anti-DLL3 antibody can be assayed by, for example, a method described in Examples.

The antibody having an internalization activity can be conjugated with, for example, the cytotoxic substance and used as a pharmaceutical composition such as an anticancer agent described later.

### Preparation of Anti-DLL3 Antibody

### 1. Preparation of Anti-DLL3 Antibody Using Monoclonal Antibody-Producing Hybridoma

Monoclonal antibody-producing hybridomas can be prepared according to a technique known in the art as follows: first, animals are immunized with DLL3 proteins or partial peptides thereof (which will be described later) used as sensitizing antigens according to a usual immunization method. The obtained immunocytes are fused with parental cells known in the art by a usual cell fusion method to obtain hybridomas. These hybridomas are further screened for cells producing the antibody of interest by a usual screening method to select hybridomas producing the anti-DLL3 antibody. The desired anti-DLL3 monoclonal antibody is obtained from the selected hybridomas. Specifically, the anti-DLL3 monoclonal antibody is prepared as follows:

### (1) Preparation of DLL3 Protein

First, DLL3 genes can be expressed to obtain DLL3 proteins used as sensitizing antigens for antibody obtainment. Specifically, the DLL3-encoding gene sequence is inserted into expression vectors known in the art, with which appropriate host cells are then transformed. Then, the human DLL3 proteins of interest are purified from the host cells or a culture supernatant thereof by a method known in the art. Purified natural DLL3 proteins or fusion proteins comprising the desired partial polypeptide of the DLL3 protein fused with a different polypeptide may be used as immunogens. For example, antibody Fc fragments, peptide tags, and so on can be used for producing the fusion proteins used as immunogens. Expression vectors for the fusion proteins can be prepared by fusing, in frame, two or more genes respectively encoding the desired polypeptide fragments and inserting this fusion gene into expression vectors. The method for preparing the fusion proteins is described in Molecular Cloning 2nd ed. (Sambrook, J. et al., Molecular Cloning 2nd ed., 9.47-9.58, Cold Spring Harbor Lab. Press, 1989).

The DLL3 proteins thus purified can be used as sensitizing antigens for the immunization of mammals. Partial peptides of DLL3 can also be used as sensitizing antigens. For example, the following peptides can be used as sensitizing antigens:

The region and size of the partial peptide of DLL3 used are not limited. The number of amino acids constituting the peptide serving as a sensitizing antigen is preferably at least 3 or more, for example, 5 or more or 6 or more. More specifically, peptides of 8 to 50 residues, preferably 10 to 30 residues can be used as sensitizing antigens.

(2) Immunization with DLL3 Protein

Mammals are immunized with the DLL3 proteins or partial peptides thereof as sensitizing antigens. The immunized mammals are not particularly limited. For obtaining the monoclonal antibody by the cell fusion method, it is preferred that the immunized animals should be selected in consideration of compatibility with the parental cells used in cell fusion. In general, rodents are preferable as the immunized animals. Specifically, mice, rats, hamsters, or rabbits can be used as the immunized animals. In addition, monkeys or the like may be used as the immunized animals.

These animals can be immunized with the sensitizing antigens according to a method known in the art. For example, a general method can involve immunizing the mammals with the sensitizing antigens by intraperitoneal or subcutaneous injection. Specifically, the sensitizing antigens are administered to the mammals several times at 4- to 21-day intervals. The sensitizing antigens are diluted with PBS (phosphate-buffered saline), saline, or the like at an appropriate dilution ratio and used in the immunization. Furthermore, the sensitizing antigens may be administered together with an adjuvant. For example, the antigens can be mixed with a Freund's complete adjuvant for emulsification to prepare sensitizing antigens. Moreover, an appropriate carrier can be used in the immunization with the sensitizing antigens. Particularly, when partial peptides having a small molecular weight are used as the sensitizing antigens, it is preferred that the sensitizing antigen peptides should be bound to carrier proteins such as albumin or keyhole limpet hemocyanin and used in the immunization.

### (3) DNA Immunization

The monoclonal antibody can also be obtained by DNA immunization. The DNA immunization is an immunostimulation method involving: immunizing animals by the administration of vector DNA that has been constructed in a form capable of expressing antigenic protein-encoding genes in the immunized animals; and allowing the immunized animals to express the immunizing antigens in vivo. The DNA immunization can be expected to be superior to general immunization methods using the administration of protein antigens as follows:
it can provide immunostimulation with membrane protein (e.g., DLL3) structures maintained; and
it eliminates the need of purifying immunizing antigens.

For obtaining the monoclonal antibody of the present invention by the DNA immunization, first, animals are immunized by the administration of DLL3 protein expression vector DNA. DLL3-encoding DNA can be synthesized by a method known in the art such as PCR. The obtained DNA is inserted into appropriate expression vectors, with which animals are immunized by administration. For example, commercially available expression vectors such as pcDNA3.1 can be used as the expression vectors. Likewise, a method generally used can be used for administering the vectors to the animals. For example, gold particles with the expression vectors adsorbed onto can be inserted into cells using a gene gun to perform DNA immunization.

(4) Preparation of Hybridoma A rise in the amount of the desired antibody is confirmed in the serum of the mammals thus immunized. Then, immunocytes are collected from the mammals and subjected to cell fusion. Particularly, spleen cells can be used as preferable immunocytes.

Mammalian myeloma cells are used in the cell fusion with the immunocytes. It is preferred that the myeloma cells should have an appropriate selection marker for screening. The selection marker refers to a character that can survive (or cannot survive) under particular culture conditions. For example, hypoxanthine-guanine phosphoribosyltransferase deficiency (hereinafter, abbreviated to HGPRT deficiency) or thymidine kinase deficiency (hereinafter, abbreviated to TK deficiency) is known in the art as the selection marker. Cells having the HGPRT or TK deficiency are sensitive to hypoxanthine-aminopterin-thymidine (hereinafter, abbreviated to HAT-sensitive). The HAT-sensitive cells are killed in a HAT selective medium because they cannot synthesize DNA. By contrast, these cells, when fused with normal cells, can grow even in the HAT selective medium because they can continue DNA synthesis by use of the salvage pathway of the normal cells.

The cells having the HGPRT or TK deficiency can be selected in a medium containing 6-thioguanine or 8-azaguanine (hereinafter, abbreviated to 8AG) for the HGPRT deficiency or 5′-bromodeoxyuridine for the TK deficiency. The normal cells are killed in such a medium because they incorporate these pyrimidine analogs into their DNAs. By contrast, the cells deficient in these enzymes can survive in the selective medium because they cannot incorporate the pyrimidine analogs therein. In addition, a selection marker called G418 resistance imparts, to cells, 2-deoxystreptamine antibiotic (gentamicin analog) resistance via a neomycin resistance gene. Various myeloma cells suitable for the cell fusion are known in the art. For example, the following myeloma cells can be used in the production of the monoclonal antibody according to the present invention:
P3 (P3x63Ag8. 653) (J. Immunol. (1979) 123, 1548-1550),
P3x63Ag8U. 1 (Current Topics in Microbiology and Immunology (1978) 81, 1-7),
NS-1 (Kohler. G. and Milstein, C. Eur. J. Immunol. (1976) 6, 511-519),
MPC-11 (Margulies. D. H. et al., Cell (1976) 8, 405-415), SP2/0 (Shulman, M. et al., Nature (1978) 276, 269-270), FO (de St. Groth, S. F. et al., J. Immunol. Methods (1980) 35, 1-21),
S194 (Trowbridge, I. S. J. Exp. Med. (1978) 148, 313-323),
R210 (Galfre, G. et al., Nature (1979) 277, 131-133) or the like.

Basically, the cell fusion of the immunocytes with the myeloma cells is performed according to a method known in the art, for example, the method of Kohler and Milstein et al. (Kohler. G. and Milstein, C., Methods Enzymol. (1981) 73, 3-46).

More specifically, the cell fusion can be performed, for example, in a usual nutrient culture medium in the presence of a cell fusion promoter. For example, polyethylene glycol (PEG) or hemagglutinating virus of Japan (HVJ) can be used as the fusion promoter. Furthermore, an auxiliary such as dimethyl sulfoxide can also be added thereto, if desired, for enhancing fusion efficiency.

The ratio between the immunocytes and the myeloma cells used can be set arbitrarily. For example, it is preferred that the amount of the immunocytes should be set to 1 to 10 times that of the myeloma cells. For example, an RPMI1640 or MEM culture medium suitable for the growth of the myeloma cell line as well as a usual culture medium used in this kind of cell culture can be used as the culture medium in the cell fusion. Furthermore, a solution supplemented with serum (e.g., fetal calf serum (FCS)) can be added to the culture medium.

For the cell fusion, the immunocytes and the myeloma cells are well mixed in the predetermined amounts in the culture medium and then mixed with a PEG solution preheated to approximately 37° C. to form the fusion cells (hybridomas) of interest. In the cell fusion method, for example, PEG with an average molecular weight on the order of 1000 to 6000 can usually be added at a concentration of 30 to 60% (w/v). Subsequently, the appropriate culture medium exemplified above is sequentially added to the hybridomas, and the mixture is centrifuged, followed by removal of the supernatant. This procedure is repeated to remove the cell fusion agents or the like unfavorable for hybridoma growth.

The hybridomas thus obtained can be selected by use of a selective culture medium appropriate for the selection marker of the myeloma cells used in the cell fusion. For example, the cells having the HGPRT or TK deficiency can be selected by culturing the hybridomas in a HAT culture medium (culture medium containing hypoxanthine, aminopterin, and thymidine). Specifically, when HAT-sensitive myeloma cells are used in the cell fusion, only cells successfully fused with normal cells can be grown selectively in the HAT culture medium. The culture using the HAT culture medium is continued for a time long enough to kill cells (non-fused cells) other than the hybridomas of interest. Specifically, the culture can generally be performed for a few days to a few weeks to select the hybridomas of interest. Subsequently, hybridomas producing the antibody of interest can be screened for and cloned as single clones by a usual limiting dilution method.

The screening of the antibody of interest and cloning as single clones thereof can be performed preferably by a screening method based on antigen-antibody reaction known in the art. For example, the antigens are bound to a carrier such as beads made of polystyrene or the like or a commercially available 96-well microtiter plate and reacted with the culture supernatant of the hybridomas. Subsequently, the carrier is washed and then reacted with enzyme-labeled secondary antibodies or the like. When the culture supernatant contains the antibody of interest reactive with the sensitizing antigens, the secondary antibodies bind to the carrier via this antibody. Finally, the secondary antibodies bound with the carrier can be detected to determine the presence of the antibody of interest in the culture supernatant. The hybridomas producing the desired antibody capable of binding to the antigen can be cloned by a limiting dilution method or the like. In this screening, the DLL3 proteins used in the immunization or DLL3 proteins substantially identical thereto can be used preferably as the antigens. For example, cell lines expressing DLL3, soluble DLL3, or the like can be used as the antigens.

A method described in International Publication No. WO 03/104453 may be used in the production of the antibody against human DLL3.

Moreover, in addition to the method for obtaining the hybridomas by immunizing non-human animals with the antigens, human lymphocytes may be sensitized with the antigens to obtain the antibody of interest. Specifically, the human lymphocytes are first sensitized with the DLL3 proteins in vitro. Subsequently, the sensitized lymphocytes are fused with appropriate fusion partners. For example, human-derived myeloma cells capable of dividing throughout their lives can be used as the fusion partners (see Japanese Patent Publication No. 1-59878).

Furthermore, the anti-DLL3 human antibody can also be obtained by administering the DLL3 proteins as antigens to transgenic animals having all repertoires of human antibody genes or by immunizing the animals with DNA that has been constructed to express DLL3 in the animals. Antibody-producing cells from the immunized animals can be immortalized by treatment such as cell fusion with appropriate fusion partners or infection with Epstein-Barr virus. From the immortalized cells thus obtained, human antibodies against the DLL3 protein can be isolated (see International Publication Nos. WO 94/25585, WO 93/12227, WO 92/03918, and WO 94/02602). Furthermore, the immortalized cells can also be cloned as cells producing antibodies having the reaction specificity of interest. When transgenic animals are used as the immunized animals, the immune systems of the animals recognize human DLL3 as foreigners. Thus, the human antibodies against human DLL3 can be obtained easily.

(5) Obtainment of Monoclonal Antibody from Hybridoma

The monoclonal antibody-producing hybridomas thus prepared can be subcultured in a usual culture medium. Moreover, the hybridomas can also be stored over a long period in liquid nitrogen.

The hybridomas are cultured according to a usual method, and the monoclonal antibody of interest can be obtained from the culture supernatant thereof. Alternatively, the hybridomas are administered to mammals compatible therewith and grown, and the monoclonal antibody can also be obtained in the form of ascitic fluids. The former method is suitable for obtaining highly pure antibodies.

### 2. Preparation of Anti-DLL3 Antibody by Genetic Engineering Approach

### (1) Cloning of Antibody Gene

The antibody may be prepared by a genetic engineering approach using antibody genes cloned from antibody-producing cells. The cloned antibody genes can be incorporated into appropriate vectors and expressed as antibodies by the transformation of hosts. Methods for the antibody gene isolation, the introduction into vectors, and the transformation of host cells have already been established (see e.g., Vandamme, A. M. et al., Eur. J. Biochem. (1990) 192, 767-775).

For example, cDNAs encoding the variable regions of the anti-DLL3 antibody can be obtained from the anti-DLL3 antibody-producing hybridoma cells. For this purpose, usually, total RNAs are first extracted from the hybridomas. For example, the following methods can be used for mRNA extraction from the cells:
guanidine ultracentrifugation method (Chirgwin, J. M. et al., Biochemistry (1979) 18, 5294-5299), and
AGPC method (Chomczynski, P. et al., Anal. Biochem. (1987) 162, 156-159).

The extracted mRNAs can be purified using mRNA Purification Kit (manufactured by GE Healthcare Bio-Sciences Corp.) or the like. Alternatively, a kit for directly extracting total mRNAs from cells is also commercially available, such as QuickPrep mRNA Purification Kit (manufactured by GE Healthcare Bio-Sciences Corp.). The total mRNAs may be obtained from the hybridomas using such a kit. From the obtained mRNAs, antibody variable region-encoding cDNAs can be synthesized using reverse transcriptase. In this procedure, arbitrary 15- to 30-base sequences selected from sequences common to the antibody genes can be used as primers. The cDNAs can be synthesized using AMV Reverse Transcriptase First-strand cDNA Synthesis Kit (manufactured by Seikagaku Corp.) or the like. Moreover, 5′-Ampli FINDER RACE Kit (manufactured by Clontech Laboratories, Inc.) and 5′-RACE PCR (Frohman, M. A. et al., Proc. Natl. Acad. Sci. USA (1988) 85, 8998-9002; and Belyavsky, A. et al., Nucleic Acids Res. (1989) 17, 2919-2932) can be used for the cDNA synthesis and amplification. Furthermore, appropriate restriction enzyme sites described later can be introduced into both ends of the cDNAs in the course of such cDNA synthesis.

From the obtained PCR products, the cDNA fragments of interest are purified and subsequently ligated with vector DNAs. The recombinant vectors thus prepared are introduced into E. coli or the like. After colony selection, the desired recombinant vectors can be prepared from E. coli that has formed the colony. Then, the cDNA can be sequenced by a method known in the art, for example, a dideoxynucleotide chain termination method.

Moreover, cDNA libraries may be used for obtaining the antibody variable region-encoding genes. First, cDNAs are synthesized with mRNAs extracted from the antibody-producing cells as templates to obtain cDNA libraries. A commercially available kit is conveniently used in the cDNA library synthesis. In actuality, mRNAs from only a small number of cells are obtained in very small amounts. Therefore, direct purification thereof results in low yields. Thus, carrier RNAs shown to be free from the antibody genes are usually added thereto, followed by mRNA purification. Alternatively, when RNAs can be extracted in given amounts from the antibody-producing cells, efficient extraction can be achieved without using carrier RNAs. The addition of the carrier RNAs may be unnecessary for RNA extraction from, for example, 10 or more or 30 or more, preferably 50 or more antibody-producing cells.

The antibody genes are amplified by PCR with the obtained cDNA libraries as templates. Primers for the PCR amplification of the antibody genes are known in the art. For example, primers for human antibody gene amplification can be designed based on the disclosure of the paper (J. Mol. Biol. (1991) 222, 581-597) or the like. These primers have a nucleotide sequence differing on an immunoglobulin subclass basis. Thus, when cDNA libraries whose subclass is unknown are used as templates, PCR is performed by selecting primers in consideration of every possibility.

Specifically, for example, for the purpose of obtaining human IgG-encoding genes, primers can be used, which are capable of amplifying each of genes encoding γ1 to γ4 heavy chains and κ and λ light chains. Primers annealing to a portion corresponding to the hinge region are generally used as 3′ primers for amplifying IgG variable region genes. On the other hand, primers appropriate for each subclass can be used as 5′ primers.

The PCR products obtained from the primers for gene amplification for these heavy and light chain subclasses are prepared as their respective independent libraries. The libraries thus synthesized can be used to reshape immunoglobulins comprising the heavy and light chains in combination. The antibody of interest can be screened for with the binding activities of the reshaped immunoglobulins for DLL3 as an index.

(2) Introduction of Antibody Gene into Host Cell

For producing the anti-DLL3 antibody, the cloned antibody genes can be incorporated into expression vectors such that these genes are expressed under the control of expression control regions. The expression control regions for antibody expression encompass, for example, enhancers and promoters. Subsequently, appropriate host cells can be transformed with these expression vectors to obtain recombinant cells expressing the anti-DLL3 antibody-encoding DNA.

For the antibody gene expression, the antibody heavy chain- and light chain-encoding DNAs can be incorporated separately in different expression vectors. The same host cell can be co-transfected with the heavy chain- and light chain-incorporated vectors and thereby allowed to express antibody molecules comprising the heavy and light chains. Alternatively, the heavy chain- and light chain-encoding DNAs may be incorporated in single expression vectors, with which host cells are transformed (see International Publication No. WO 94/11523).

Many combinations of hosts and expression vectors are known in the art for introducing the isolated antibody genes into appropriate hosts for antibody preparation. All of these expression systems can be applied to the present invention. When eukaryotic cells are used as the hosts, animal, plant, or fungus cells can be used. Specifically, examples of the animal cells that can be used in the present invention can include the following cells: i) mammalian cells such as CHO, COS, myeloma, BHK (baby hamster kidney), Hela, Vero, HEK293, Ba/F3, HL-60, Jurkat, and SK-HEP1 cells;
ii) amphibian cells such as Xenopus oocytes; and
iii) insect cells such as: sf9, sf21, and Tn5 cells.

For the plant cells, antibody gene expression systems are known in the art, which involve cells derived from the genus Nicotiana (e.g., Nicotiana tabacum). Cultured callus cells can be used in the plant cell transformation.

Furthermore, the following cells can be used as the fungus cells:
cells derived from yeasts such as the genus Saccharomyces (e.g., Saccharomyces cerevisiae) and filamentous fungi of the genus Pichia (e.g., Pichia pastoris), and cell derived from the genus Aspergillus (e.g., Aspergillus niger).

Alternatively, antibody gene expression systems using prokaryotic cells are also known in the art. For example, when bacterial cells are used, bacterial cells derived from E. coli, Bacillus subtilis, or the like can be used in the present invention.

For the gene expression using mammalian cells, a useful promoter routinely used, the antibody gene to be expressed, and a poly A signal located 3′-downstream thereof can be ligated functionally. Examples of the promoter/enhancer can include a human cytomegalovirus immediate early promoter/enhancer.

In addition, for example, virus promoters/enhancers or mammalian cell-derived promoters/enhancers (e.g., human elongation factor 1α (HEF1α)) can be used in the antibody expression. Examples of the viruses whose promoter/enhancer can be used can specifically include retrovirus, polyomavirus, adenovirus, and simian virus 40 (SV40).

The SV40 promoter/enhancer can be used according to the method of Mulligan et al. (Nature (1979) 277, 108). Moreover, the HEF1α promoter/enhancer can be used easily in the gene expression of interest by the method of Mizushima et al. (Nucleic Acids Res. (1990) 18, 5322).

When antibodies are produced using animal cells, the signal sequence of the heavy or light chain gene of the antibody is preferably used as a signal sequence required for extracellular secretion. Moreover, the signal sequence of a secretory protein such as IL-3 or IL-6 may be used.

For the gene expression using E. coli, a useful promoter routinely used, a signal sequence for antibody secretion, and the antibody gene to be expressed can be ligated functionally. Examples of the promoter can include lacZ and araB promoters. The lacZ promoter can be used according to the method of Ward et al. (Nature (1989) 341, 544-546; and FASEBJ. (1992) 6, 2422-2427). Alternatively, the araB promoter can be used in the gene expression of interest by the method of Better et al. (Science (1988) 240, 1041-1043).

When antibodies are produced in E. coli periplasm, a pelB signal sequence (Lei, S. P. et al., J. Bacteriol. (1987) 169, 4379) may be used for antibody secretion. Then, the antibodies produced in the periplasm are separated and then refolded by use of protein denaturants such as urea and guanidine hydrochloride such that the resultant antibodies have the desired binding activity.

A replication origin derived from SV40, polyomavirus, adenovirus, bovine papillomavirus (BPV), or the like can be inserted in the expression vectors. Furthermore, a selection marker can be inserted in the expression vectors for increasing a gene copy number in the host cell systems. Specifically, selection markers can be used, such as aminoglycoside phosphotransferase (APH) gene, thymidine kinase (TK) gene, E. coli xanthine-guanine phosphoribosyltransferase (Ecogpt) gene, and dihydrofolate reductase (dhfr) gene.

(3) Obtainment of Antibody from Host Cell

The host cells are transformed with these expression vectors, and the transformed host cells are then cultured in vitro or in vivo to produce the antibody of interest. The culture of the host cells is performed according to a method known in the art. For example, a DMEM, MEM, RPMI1640, or IMDM culture medium can be used and may be used in combination with a solution supplemented with serum such as fetal calf serum (FCS).

The antibodies thus expressed and produced can be purified by using, alone or in appropriate combination, usual protein purification methods known in the art. For example, affinity or chromatography columns (e.g., protein A columns), filters, ultrafiltration, salting-out, and dialysis can be selected and combined appropriately to separate and purify the antibodies (Antibodies A Laboratory Manual. Ed Harlow, David Lane, Cold Spring Harbor Laboratory, 1988).

Thus, the present invention provides a gene encoding the antibody of the present invention. The present invention also provides a vector comprising the gene. The present invention further provides a host cell carrying the vector. The present invention further provides a method for producing an antibody encoded by the gene, comprising the step of culturing the host cell.

### 3. Antibody Production by Transgenic Animal

In addition to the host cells, transgenic animals can also be used in the recombinant antibody production. Specifically, the antibody of interest can be obtained from animals transfected with the genes encoding this antibody of interest. For example, the antibody genes can be inserted in frame into genes encoding proteins specifically produced in milk to construct fusion genes. For example, goat β casein can be used as the proteins secreted into milk. DNA fragments containing the fusion genes having the antibody gene insert are injected into goat embryos, which are in turn introduced into female goats. From milk produced by transgenic goats (or progeny thereof) brought forth by the goats that have received the embryos, the desired antibody can be obtained as a fusion protein with the milk protein. Moreover, in the transgenic goats, hormone can be used appropriately for increasing the amount of milk containing the desired antibody produced from the transgenic goats (Ebert, K. M. et al., Bio/Technology (1994) 12, 699-702).

### Pharmaceutical Composition

Since DLL3 is highly expressed in small-cell lung cancer tissues, the anti-DLL3 antibody has a cancer cell-specific cytotoxic activity. Thus, the anti-DLL3 antibody is useful in the treatment of cancer expressing DLL3.

Specifically, the present invention provides a pharmaceutical composition comprising an antibody which binds to DLL3 protein as an active ingredient. In an embodiment, the pharmaceutical composition is a cell growth inhibitor, particularly, an anticancer agent. Preferably, the cell growth inhibitor and the anticancer agent of the present invention are administered to a subject having cancer or possibly having cancer.

The anti-DLL3 antibody used in the pharmaceutical composition (e.g., anticancer agent) of the present invention is not particularly limited, and, for example, any of the anti-DLL3 antibodies described above can be used.

In the present invention, the phrase “comprising the antibody which binds to DLL3 as an active ingredient” means comprising the anti-DLL3 antibody as a main active ingredient and does not limit the content of the anti-DLL3 antibody.

The pharmaceutical composition of the present invention may comprise a cytotoxic substance-conjugated anti-DLL3 antibody as an active ingredient. This pharmaceutical composition can be used as, for example, a cell growth inhibitor, particularly, an anticancer agent. Preferably, the cell growth inhibitor and the anticancer agent of the present invention are administered to a subject having cancer or possibly having cancer.

In the present invention, the phrase “comprising the cytotoxic substance-conjugated anti-DLL3 antibody as an active ingredient” means comprising the cytotoxic substance-conjugated anti-DLL3 antibody as a main active ingredient and does not limit the content of the cytotoxic substance-conjugated anti-DLL3 antibody.

When the disease targeted by the pharmaceutical composition of the present invention is cancer, the targeted cancer is not particularly limited and is preferably lung cancer, particularly, small-cell lung cancer. The cancer may be any of primary foci and metastatic foci.

The pharmaceutical composition of the present invention can be administered either orally or parenterally to a patient. Parenteral administration is preferable. Specific examples of such an administration method include injection, transnasal, pulmonary, and transdermal administrations. Examples of the injection administration include intravenous, intramuscular, intraperitoneal, and subcutaneous injections, through which the pharmaceutical composition of the present invention can be administered systemically or locally. Moreover, the administration method can be selected appropriately according to the age or symptoms of the patient. The dose of the pharmaceutical composition of the present invention can be selected from among a dose range of, for example, 0.0001 mg to 1000 mg per kg body weight per dosing. Alternatively, the dose can be selected from among a range of, for example, 0.001 to 100000 mg per body. However, the pharmaceutical composition of the present invention is not limited to these doses.

The pharmaceutical composition of the present invention can be formulated according to a standard method (e.g., Remington's Pharmaceutical Science, latest edition, Mark Publishing Company, Easton, U.S.A) and may additionally contain pharmaceutically acceptable carriers or additives. Examples thereof include, but not limited thereto, surfactants, excipients, coloring agents, flavoring agents, preservatives, stabilizers, buffers, suspending agents, tonicity agents, binders, disintegrants, lubricants, flow promoters, and corrigents. Other carriers routinely used can be used appropriately. Specific examples of the carriers can include light anhydrous silicic acid, lactose, crystalline cellulose, mannitol, starch, carmellose calcium, carmellose sodium, hydroxypropylcellulose, hydroxypropylmethylcellulose, polyvinyl acetal diethylaminoacetate, polyvinyl pyrrolidone, gelatin, middle chain fatty acid triglyceride, polyoxyethylene hydrogenated castor oil 60, white sugar, carboxymethylcellulose, corn starch, and inorganic salts.

Upon contact with DLL3-expressing cells, the anti-DLL3 antibody of the present invention can damage the DLL3-expressing cells or inhibit their growth. Such a method using the anti-DLL3 antibody is also incorporated in the scope of the present invention. The antibody used is not particularly limited, and, for example, any of the antibodies described above can be used. The cells to which the anti-DLL3 antibody binds are not particularly limited as long as the cells express DLL3. In the present invention, the DLL3-expressing cells are preferably cancer cells, more preferably lung cancer cells, particularly preferably small-cell lung cancer cells.

In the present invention, the “contact” is performed, for example, by adding the antibody to a culture medium of DLL3-expressing cells cultured in vitro. In the present invention, the “contact” is also performed by administering the anti-DLL3 antibody to non-human animals implanted with DLL3-expressing cells in their bodies or to animals endogenously having cancer cells expressing DLL3.

Methods shown below are preferably used for evaluating or determining cytotoxicity caused in the DLL3-expressing cells by the contact of the anti-DLL3 antibody. Examples of the methods for evaluating or determining the cytotoxic activity in vitro can include the ADCC or CDC activity assay described above. Whether or not the anti-DLL3 antibody has an ADCC activity or has a CDC activity can be determined by a method known in the art (e.g., Current protocols in Immunology, Chapter 7. Immunologic studies in humans, Editor, John E, Coligan et al., John Wiley & Sons, Inc., (1993)). In the activity assay, antibodies that have an isotype identical to that of the anti-DLL3 antibody and do not bind to the cells are used as control antibodies in the same way as in the anti-DLL3 antibody. When the anti-DLL3 antibody exhibits a stronger cytotoxic activity than that of the control antibodies, the anti-DLL3 antibody can be determined to have the activity.

The isotype of an antibody is defined based on the sequence of the heavy chain constant region in the amino acid sequence of this antibody. The antibody isotype is finally determined depending on class switching caused by genetic recombination on the chromosome during the maturation of antibody-producing B cells in vivo. Difference in isotype reflects difference between the physiological/pathological functions of antibodies. Specifically, it is known that, for example, the strength of the cytotoxic activity is influenced not only by antigen expression levels but by antibody isotypes. Thus, for the cytotoxic activity assay described above, it is preferred that the antibodies used as controls should have an isotype identical to that of the analyte antibody.

Moreover, for evaluating or determining the cytotoxic activity in vivo, for example, DLL3-expressing cancer cells are intradermally or subcutaneously transplanted to non-human test animals. Then, the analyte antibody is intravenously or intraperitoneally administered thereto on a daily basis or at a few day-intervals from the administration day or the next day. The cytotoxic activity can be determined by measuring tumor sizes over time. Control antibodies having an isotype identical thereto are administered in the same way in the in vitro evaluation. When the anti-DLL3 antibody-administered group has a significantly smaller tumor size than that of the control antibody-administered group, the anti-DLL3 antibody can be determined to have the cytotoxic activity. When mice are used as the non-human test animals, nude (nu/nu) mice can be used preferably, which are genetically deficient in thymus gland and thus lack the functions of T lymphocytes. The use of these mice can exclude the involvement of the endogenous T lymphocytes of the test animals in the evaluation/determination of the cytotoxic activity of administered antibodies.

### Diagnostic Drug (Diagnosis Method)

The present invention also provides a method for diagnosing cancer, comprising detecting the DLL3 protein or a gene encoding the DLL3 protein. DLL3 expression has been confirmed to be remarkably increased in cancer tissues or cancer cell lines. Thus, DLL3 is useful as a marker for specific detection of cancer.

One specific example of the diagnosis method of the present invention can include a method for diagnosing cancer, comprising the following steps:
(a) providing a sample isolated from a test subject; and
(b) detecting the expression level of DLL3 protein or DLL3 gene in the sample.

The method of the present invention may further comprise the step of
(c) evaluating the possibility that the test subject has cancer, based on the expression level of the DLL3 protein or the DLL3 gene.

### Detection of DLL3 Protein or Gene Encoding DLL3 Protein

In one aspect of the method of the present invention, cancer is diagnosed by detecting the DLL3 protein in a sample. It is preferred that the DLL3 protein detection should be performed using an antibody that recognizes the DLL3 protein.

In the present invention, the detection encompasses quantitative or qualitative detection. Examples of the qualitative detection can include the following assays:
assay to simply determine the presence or absence of the DLL3 protein,
assay to determine the presence or absence of more than a predetermined amount of the DLL3 protein, and
assay to compare the amount of the DLL3 protein with that contained in another sample (e.g., a control sample).

On the other hand, examples of the quantitative detection can include measurement of a DLL3 protein concentration and measurement of the amount of the DLL3 protein.

The test sample according to the present invention is not particularly limited as long as the sample is likely to contain the DLL3 protein. Specifically, samples collected from living bodies such as mammals are preferable. Samples collected from humans are more preferable. Specific examples of the test sample can include blood, interstitial fluid, plasma, extravascular fluid, cerebrospinal fluid, synovial fluid, pleural fluid, serum, lymph, saliva, urine, and tissues. The sample is preferably a sample obtained from the test sample, such as a preparation in which tissues or cells collected from a living body are fixed, or a cell culture medium.

The cancer diagnosed by the present invention may be any cancer without particular limitations. Specific examples thereof can include lung cancer, particularly, small-cell lung cancer. In the present invention, any of primary foci and metastatic foci of these cancers can be diagnosed.

In the present invention, when the protein is detected in the test sample, cancer is diagnosed with its level as an index. Specifically, when the amount of the DLL3 protein detected in the test sample is larger than that of a negative control or a healthy individual, the test subject is shown to have cancer or highly possibly have cancer in the future. Specifically, the present invention relates to a method for diagnosing cancer, comprising the following steps:

(1) detecting the expression level of DLL3 in a biological sample collected from a test subject, and

(2) comparing the expression level of DLL3 detected in step (1) with that of a control, wherein when the expression level of DLL3 is higher than that of the control, the test subject is determined to have cancer.

In the present invention, the control refers to a reference sample for comparison and encompasses negative controls and biological samples of healthy individuals. The negative controls can be obtained by collecting biological samples of healthy individuals and mixing them, if necessary. The expression level of DLL3 in the control can be detected in parallel with the detection of the expression level of DLL3 in the biological sample of the test subject. Alternatively, the expression level of DLL3 in a large number of biological samples of healthy individuals can be detected in advance to statistically determine the standard expression level in the healthy individuals. Specifically, for example, mean±2×standard deviation (S. D.) or mean±3×standard deviation (S. D.) can also be used as the standard value. Statistically, the mean±2×standard deviation (S. D.) and the mean±3×standard deviation (S. D.) include values of 80% and 90% of the healthy individuals, respectively.

Alternatively, the expression level of DLL3 in the control can be set using an ROC curve. The ROC curve, or receiver operating characteristic curve, is a graph showing detection sensitivity in the ordinate and false positive rates (i.e., “1-specificity”) in the abscissa. In the present invention, the ROC curve can be obtained by plotting changes in sensitivity and false positive rate at a series of varying reference values for determining the expression level of DLL3 in biological samples.

The “reference value” for obtaining the ROC curve is a numeric value temporarily used for statistical analysis. In general, the “reference value” for obtaining the ROC curve is serially varied within a range which can cover all selectable reference values. For example, the reference value can be varied between the minimal and maximal measured values of DLL3 in a population to be analyzed.

A standard value that can be expected to offer the desired detection sensitivity and precision can be selected based on the obtained ROC curve. The standard value statistically set based on the ROC curve or the like is also called a cut-off value. In a method for detecting cancer based on the cut-off value, step (2) described above comprises comparing the expression level of DLL3 detected in step (1), with the cut-off value. Then, when the expression level of DLL3 detected in step (1) is higher than the cut-off value, cancer is detected in the test subject.

In the present invention, the expression level of DLL3 can be determined by an arbitrary method. Specifically, the expression level of DLL3 can be determined by evaluating the amount of DLL3 mRNA, the amount of the DLL3 protein, or the biological activity of the DLL3 protein. The amount of the DLL3 mRNA or protein can be determined by a method as described in the present specification.

In the present invention, the test subject is particularly preferably a human. When a non-human animal is used as the test subject, the DLL3 protein to be detected is derived from this animal species.

A method for detecting the DLL3 protein contained in the test sample is not particularly limited and is preferably an immunological detection method using the anti-DLL3 antibody as exemplified below:
enzyme-linked immunosorbent assay (ELISA),
radioimmunoassay (RIA),
enzyme immunoassay (EIA),
fluoroimmunoassay (FIA),
luminescent immunoassay (LIA),
immunoprecipitation (IP),
turbidimetric immunoassay (TIA),
Western blotting (WB),
immunohistochemical (IHC) method,
single radial immunodiffusion (SRID),
dot blot, and
slot blot.

Among these approaches, the immunohistochemical (IHC) method is a preferable immunological assay method for diagnosing cancer, comprising the step of detecting DLL3 proteins in sections in which tissues or cells obtained from a patient having cancer are fixed. The immunological methods described above, such as the immunohistochemical (IHC) method, are generally known by those skilled in the art.

Since DLL3 is a membrane protein whose expression is enhanced in a cancer cell-specific manner, cancer cells or cancer tissues can be detected using the anti-DLL3 antibody. Cancer cells contained in cells or tissues collected from living bodies are detected by the immunohistological analysis.

In another preferable aspect, cancer tissues can also be detected in vivo using the anti-DLL3 antibody. This method specifically comprises the steps of: (1) administering, to a test subject, a labeling material (e.g., radioisotope)-labeled antibody which binds to DLL3 protein; and (2) detecting the accumulation of the labeling material. The antibody can be labeled detectably for tracing the antibody administered into the living body. For example, the antibody can be labeled with a fluorescent or luminescent material or a radioisotope, and its in vivo behavior can be traced. The antibody labeled with the fluorescent or luminescent material can be observed using an endoscope or peritoneoscope. The localization of the antibody can be imaged by tracing the radioactivity of the radioisotope. In the present invention, the in vivo localization of the anti-DLL3 antibody represents the presence of cancer cells.

A positron-emitting nuclide can be used as the radioisotope for labeling the antibody for in vivo cancer detection. For example, the antibody can be labeled with a positron-emitting nuclide such as 18F, 55Co, 64Cu, 66Ga, 68Ga, 76Br, 89Zr, and 124I. A method known in the art (Acta Oncol. 32, 825-830, 1993) can be used in the labeling of the anti-DLL3 antibody with these positron-emitting nuclides.

The anti-DLL3 antibody labeled with the positron-emitting nuclide is administered to humans or animals. Then, radiation emitted by the radionuclide is measured non-invasively using PET (positron emission tomograph) and converted to images by a computed tomographic approach. The PET apparatus is intended to noninvasively obtain data about in vivo drug behavior or the like. The PET can quantitatively image radiation intensity as signal intensity. By such use of the PET, antigen molecules highly expressed in particular cancer can be detected without collecting samples from patients. The anti-DLL3 antibody may be radiolabeled with a short-life nuclide using a positron-emitting nuclide such as 11C, 13N, 15O, 18F, and 45Ti, in addition to the nuclides described above.

Research and development have been pursued as to, for example, techniques of producing short-life nuclides using a medical cyclotron and the nuclides described above or producing short-life radiolabeling compounds. The anti-DLL3 antibody can be labeled with various radioisotopes by these techniques. The anti-DLL3 antibody administered to patients accumulates in primary foci and metastatic foci according to the specificity of the anti-DLL3 antibody for pathological tissues at each site. When the anti-DLL3 antibody is labeled with the positron-emitting nuclide, its radioactivity can be detected to detect the presence of the primary foci and the metastatic foci based on the localization of the radioactivity. An active value of gamma radiation or positron emission of 25 to 4000 keV can be used appropriately for the diagnostic use. Moreover, therapeutic effect can also be expected by selecting an appropriate nuclide and administering the selected nuclide in larger amounts. A nuclide that provides a value of gamma radiation or positron emission of 70 to 700 keV can be used for obtaining anticancer effect attributed to radiation.

### Detection of Polynucleotide Encoding DLL3 Protein

In an alternative aspect of the method of the present invention, the expression of the DLL3 polynucleotide is detected. In the present invention, the detected polynucleotide is not particularly limited and is preferably mRNA. In the present invention, the detection encompasses quantitative or qualitative detection.

Examples of the qualitative detection can include the following assay procedures:
assay to simply determine the presence or absence of the DLL3 mRNA,
assay to determine the presence or absence of more than a predetermined amount of the DLL3 mRNA, and
assay to compare the amount of the DLL3 mRNA with that contained in another sample (e.g., a control sample).

On the other hand, examples of the quantitative detection can include measurement of a DLL3 mRNA concentration and measurement of the amount of the DLL3 mRNA.

In the present invention, an arbitrary sample likely to contain the DLL3 mRNA can be used as the test sample. Samples collected from living bodies such as mammals are preferable. Samples collected from humans are more preferable. Specific examples of the test sample can include blood, interstitial fluid, plasma, extravascular fluid, cerebrospinal fluid, synovial fluid, pleural fluid, serum, lymph, saliva, urine, and tissues. The sample is preferably a sample obtained from the test sample, such as a preparation in which tissues or cells collected from a living body are fixed, or a cell culture medium. These samples are encompassed by the test sample of the present invention.

In situ hybridization is preferably used for the sample obtained from the test sample, such as a preparation in which tissues or cells collected from a living body are fixed, or a cell culture medium. The in situ hybridization has been evolved as an approach for confirming the presence or absence or distribution of particular DNA or RNA in cells or tissues, and the strength of its expression. This method employs the principles on which a probe nucleic acid having a nucleotide sequence complementary to an intracellular particular nucleic acid sequence has the property of specifically forming a complex. The probe is labeled in advance with a radioisotope (RI), an antigenic substance (hapten), or the like. As a result, the hybridization site can be distinguished through the detection of the label. Thus, the in situ hybridization is used in, for example, the detection of intracellular DNA or RNA, or the like. Preferably, RI can be used for the probe labeling. For example, fluorescence labeling with a nonradioactive substance such as biotin or hapten (e.g., digoxigenin) can be used more preferably. For example, a detection method by fluorescence in situ hybridization called FISH is particularly preferably used.

The cancer diagnosed in the present invention is not particularly limited. Specific examples thereof can include lung cancer, particularly, small-cell lung cancer. In the present invention, any of primary foci and metastatic foci of these cancers can be diagnosed.

In the present invention, an arbitrary animal species expressing the DLL3 gene can be used as the test subject. The test subject is particularly preferably a human. When a non-human animal species is used as the test subject, the DLL3 gene to be detected is derived from this animal species.

Hereinafter, a specific aspect of the detection method will be described. First, a sample is prepared from a test subject. Subsequently, DLL3 mRNA contained in the sample is detected. In the present invention, cDNA synthesized from the mRNA can also be detected. In the present invention, when DLL3 mRNA or DLL3-encoding cDNA is detected in the test sample, the test subject is diagnosed as possibly having cancer. For example, when the amount of the DLL3 mRNA or DLL3-encoding cDNA detected in the test sample is larger than that in negative controls or healthy individuals, the test subject is shown to have cancer or highly possible have cancer in the future.

A method for detecting the mRNA is known in the art. Specific examples of the method that can be used in the present invention include: nucleic acid hybridization using samples immobilized on a solid phase selected from gene chips, cDNA arrays, and membrane filters; RT-PCR; real-time PCR; subtraction method; differential display method; differential hybridization; and cross hybridization.

The detection method of the present invention may be automated using various automatic detectors. Such automation achieves detection of a large number of samples in a short time.

### Kit for Cancer Diagnosis

The present invention also provides a diagnostic drug or a kit for cancer diagnosis, comprising a reagent for detecting DLL3 protein in a test sample. The diagnostic drug of the present invention comprises at least the anti-DLL3 antibody.

The reagent for cancer diagnosis of the present invention can be combined with other factors used in DLL3 detection to prepare a kit for cancer diagnosis. Specifically, the present invention relates to a kit for cancer diagnosis, which comprises: an antibody which binds to DLL3; and a reagent for detecting the binding of the antibody to DLL3 and may further comprise a control sample comprising a biological sample containing DLL3. A manual for instruction of assay procedures may further be included in the kit of the present invention.

### EXAMPLES

Hereinafter, the present invention will be described more specifically with reference to Examples. However, the present invention is not intended to be limited to these Examples.

### [Example 1]Increased Transcription of DLL3 (Delta-Like 3) in Small-Cell Lung Cancer

The gene expression distribution of human DLL3 mRNA in clinical cancers, cancer cell lines, and various normal organs was analyzed using Human Exon 1.0 ST Array (Affymetrix, Inc.). In the expression analysis, the total RNAs used were derived from tumor sites in 13 cases of isolated small-cell lung cancer tissues, 3 types of small-cell lung cancer cell lines, and 49 types of normal tissues (purchased from Clontech Laboratories, Inc., Ambion, Inc., Stratagene Corp., Cell Applications, Inc., Panomics, Inc., CHEMICON, and BioChain Institute, Inc.). All of tumor sites in isolated clinical cancer tissues and cancer cell lines (purchased from ATCC) were subjected to total RNA extraction using Trizol (Invitrogen Corp.) according to the protocol included in the product. The experiment of gene expression analysis was conducted using 1 μg of each total RNA thus obtained according to GeneChip Whole Transcript (WT) Sense Target Labeling Assay Manual (Affymetrix, Inc.). Human Exon 1.0 ST Array Data was digitized using ExACT (Exon Array Computational Tool) software provided by Affymetrix, Inc.

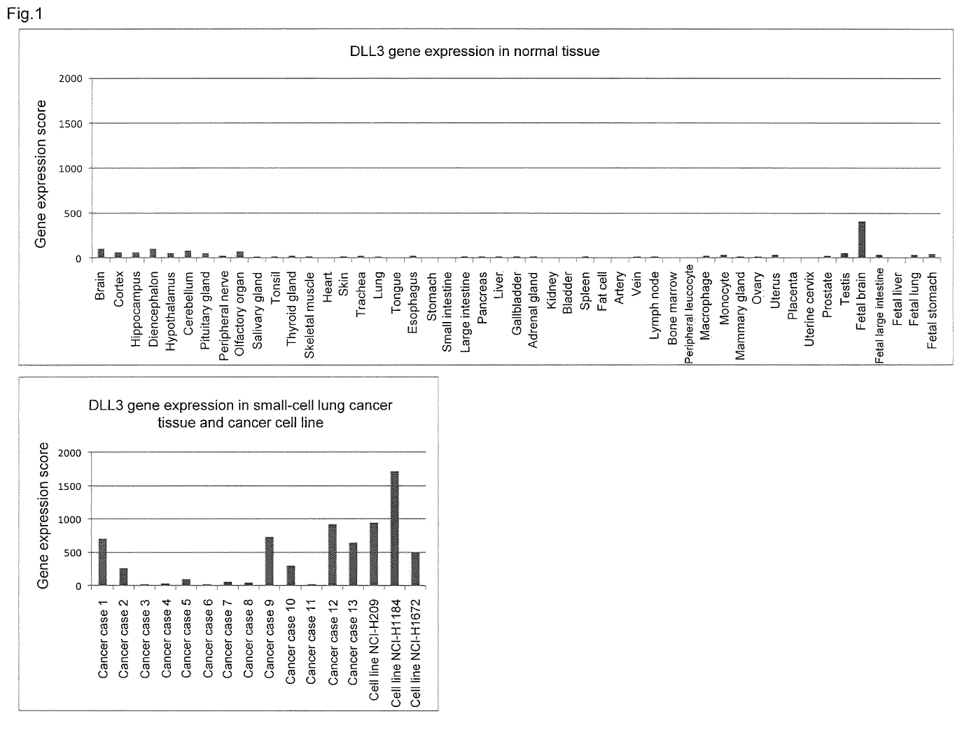

13 core probe sets for DLL3 were present on Human Exon 1.0 ST Array. The mean of expression values was determined from these probe sets, and the gene expression level was compared among tissues. The expression data obtained from the normal tissues, the tumor sites in isolated small-cell lung cancer tissues, and the small-cell lung cancer cell lines is shown in FIG. 1.

It was found that human DLL3 gene expression was remarkably increased in the tumor sites in isolated small-cell lung cancer tissues and the small-cell lung cancer cell lines, compared with the normal tissues except for the fetal brain. These results promise the effectiveness of therapy using an antitumor agent molecularly targeting human DLL3, i.e., the possibility of reducing the size of tumor without damaging normal tissues.

### [Example 2]cDNA Cloning and Preparation of Recombinant Cell

The human DLL3 cDNA (NM_016941) as set forth in SEQ ID NOs: 1 and 57 was amplified by PCR from a human fetal brain cDNA library and cloned into expression vectors pMCN for mammals. The pMCN vector achieves the expression of a foreign gene under the control of a mouse CMV promoter (GenBank: U68299). The pMCN vector has a Geneticin resistance gene. A CHO cell line DG44 (Invitrogen Corp.) was transformed with the human DLL3 expression vector. Drug-resistant cells were selected in the presence of Geneticin. Cloned cells stably expressing the human DLL3 protein were selected using commercially available anti-DLL3 antibodies (R&D Systems, Inc., MAB4315) to establish human DLL3/DG cells. Likewise, a mouse IL-3-dependent pro B cell line Ba/F3 was transformed the human DLL3 expression vector to establish human DLL3/BaF3 cells.

The mouse DLL3 cDNA (NM_007866) as set forth in SEQ ID NOs: 2 and 58 was amplified by PCR from a mouse fetal cDNA library and cloned into expression vectors pMCN for mammals. A cell line Ba/F3 was transformed with the mouse DLL3 expression vector. Drug-resistant cells were selected in the presence of Geneticin. Cells expressing the mouse DLL3 protein were selected using commercially available antibodies (R&D Systems, Inc., MAB4315) to establish mouse DLL3/BaF3 cells.

The human DLL1 cDNA (NM_005618) as set forth in SEQ ID NOs: 3 and 59 was amplified by PCR from a human spleen cDNA library and cloned into expression vectors pMCN for mammals. A cell line Ba/F3 was transformed with the human DLL1 expression vector. Drug-resistant cells were selected in the presence of Geneticin. Cells expressing the human DLL1 protein were selected using commercially available antibodies (R&D Systems, Inc., MAB1818) to establish human DLL1/BaF3 cells.

The human Notch1 cDNA (NM_017617) as set forth in SEQ ID NOs: 4 and 60 was amplified by PCR from a breast cancer cell line DU4475 cDNA library and cloned into expression vectors pMCN for mammals. A cell line DG44 was transformed with the human Notch1 expression vector. Drug-resistant cells were selected in the presence of Geneticin. Cells expressing the human Notch1 protein were selected using commercially available antibodies (GeneTex Inc., GTX23294) to establish human Notch1/DG44 cells.

Cell lines producing a soluble DLL3 protein or its N-terminal deletion variant protein were prepared for the purpose of obtaining immunogens for anti-DLL3 antibody obtainment and determining epitopes for the obtained antibodies. The human DLL3 extracellular sequence is composed of a Notch receptor-binding motif DSL domain (Nos. 176-215 in the amino acid sequence) and six EGF-like domains (1: 216-249, 2: 274-310, 3: 312-351, 4: 353-389, 5: 391-427, and 6: 429-465).

cDNA encoding a chimeric molecule human DLL3-Fc (SEQ ID NO: 5) consisting of a human DLL3 extracellular region (27-492 in the amino acid sequence of SEQ ID NO: 1) and mouse IgG2a antibody constant region sequences was prepared and cloned into expression vectors pMCN for mammals. A cell line DG44 was transformed with the human DLL3-Fc expression vector. Drug-resistant cells were selected in the presence of Geneticin. Clones highly expressing the human DLL3-Fc protein were selected by ELISA using anti-mouse antibodies to establish human DLL3-Fc-producing DG44 cells.

cDNA encoding a chimeric molecule human DLL3delta1-Fc (SEQ ID NO: 6) consisting of a partial sequence of the human DLL3 extracellular region (176-492 in the amino acid sequence of SEQ ID NO: 1) and mouse IgG2a antibody constant region sequences was prepared and cloned into expression vectors pMCN for mammals. A cell line DG44 was transformed with the human DLL3delta1-Fc expression vector. Drug-resistant cells were selected in the presence of Geneticin. Clones highly expressing the human DLL3delta1delta1-Fc protein were selected by ELISA using anti-mouse antibodies to establish human DLL3delta1-Fc-producing DG44 cells.

cDNA encoding a chimeric molecule human DLL3delta2-Fc (SEQ ID NO: 7) consisting of a partial sequence of the human DLL3 extracellular region (216-492 in the amino acid sequence of SEQ ID NO: 1) and mouse IgG2a antibody constant region sequences was prepared and cloned into expression vectors pMCN for mammals. A cell line DG44 was transformed with the human DLL3delta2-Fc expression vector. Drug-resistant cells were selected in the presence of Geneticin. Clones highly expressing the human DLL3delta1delta2-Fc protein were selected by ELISA using anti-mouse antibodies to establish human DLL3delta2-Fc-producing DG44 cells.

cDNA encoding a chimeric molecule mouse DLL3-Fc (SEQ ID NO: 8) consisting of a mouse DLL3 extracellular region (33-490 in the amino acid sequence of SEQ ID NO: 2) and mouse IgG2a antibody constant region sequences was prepared and cloned into expression vectors pMCN for mammals. A cell line DG44 was transformed with the mouse DLL3-Fc expression vector. Drug-resistant cells were selected in the presence of Geneticin. Clones highly expressing the mouse DLL3-Fc protein were selected by ELISA using an anti-mouse antibody to establish mouse DLL3-Fc-producing DG44 cells.

Each protein of interest was purified from the culture supernatant of the Fc fusion protein-producing cell line by Protein G affinity column chromatography and gel filtration chromatography. The concentration of the purified protein was determined by DC protein assay (Bio-Rad Laboratories, Inc.) with the IgG known for its concentration as a standard.

### [Example 3]Obtainment of Anti-DLL3 Antibody and Analysis of Epitope and Antibody Internalization

Six- to seven-week-old Balb/c (Charles River Laboratories Japan, Inc.) and MRL/MpJJmsSlc-lpr/lpr (Japan SLC, Inc.) mice were immunized. For the initial challenge, by prepared into an emulsion using a Freund's complete adjuvant (Becton, Dickinson and Company), the antigenic proteins were subcutaneously administered thereto at a dose of 0.1 mg human DLL3-Fc/head. Two weeks later, an antigen emulsion prepared using a Freund's incomplete adjuvant was subcutaneously administered thereto at a dose of 0.05 mg/head a total of 3 to 6 times on a once-a-week basis. 0.05 mg of the antigenic proteins was intravenously administered to each mouse individual confirmed to have a rise in antibody titer in its serum. Three days later, their spleen cells were extracted and mixed with mouse myeloma cells P3-X63Ag8U1 (ATCC) at a cell count ratio of approximately 3:1. These cells were fused by the polyethylene glycol (PEG) method. After removal of PEG by centrifugation, the cells were suspended in an RPMI1640 medium containing 1×HAT media supplement (Sigma-Aldrich Corp.), 0.5×BM-Condimed Hi Hybridoma cloning supplement (Roche Diagnostics GmbH), and 10% fetal bovine serum to adjust the cell concentration. Then, the cells were seeded to each well of a 96-well plate. Hybridoma colony formation was confirmed, and the presence or absence of anti-DLL3 antibodies contained in the culture supernatant was then analyzed by ELISA using a plate coated with human DLL3-Fc. Hybridoma cells contained in positive wells were cloned by the limiting dilution method to establish hybridoma lines producing the anti-DLL3 antibodies. The monoclonal antibodies were isotyped using IsoStrip (Roche Diagnostics GmbH).

Each IgG monoclonal antibody was purified from the culture supernatants of the established hybridomas by Protein G affinity column chromatography and desalting treatment. The concentration of the purified antibody was determined by DC protein assay.

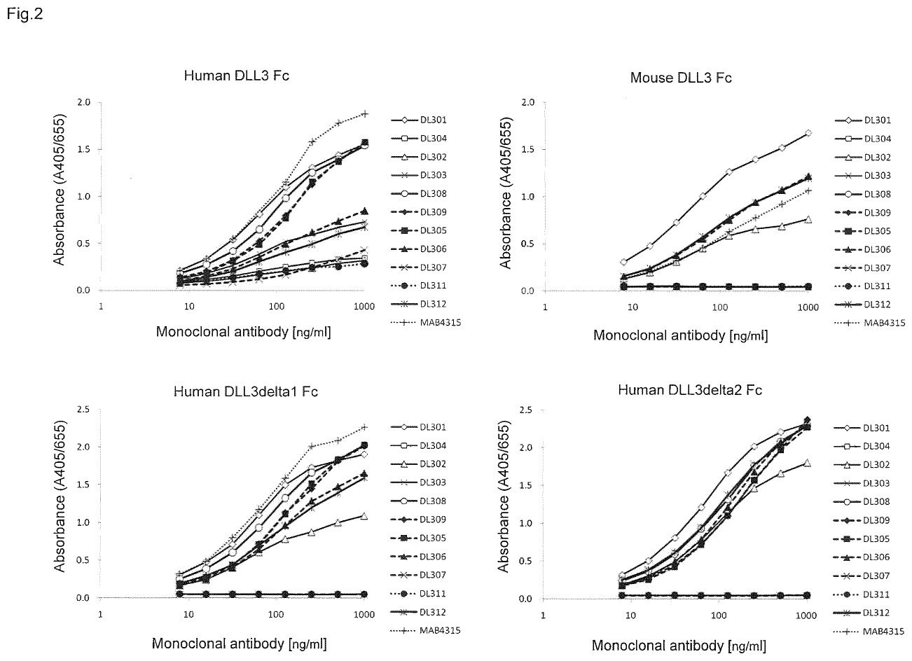

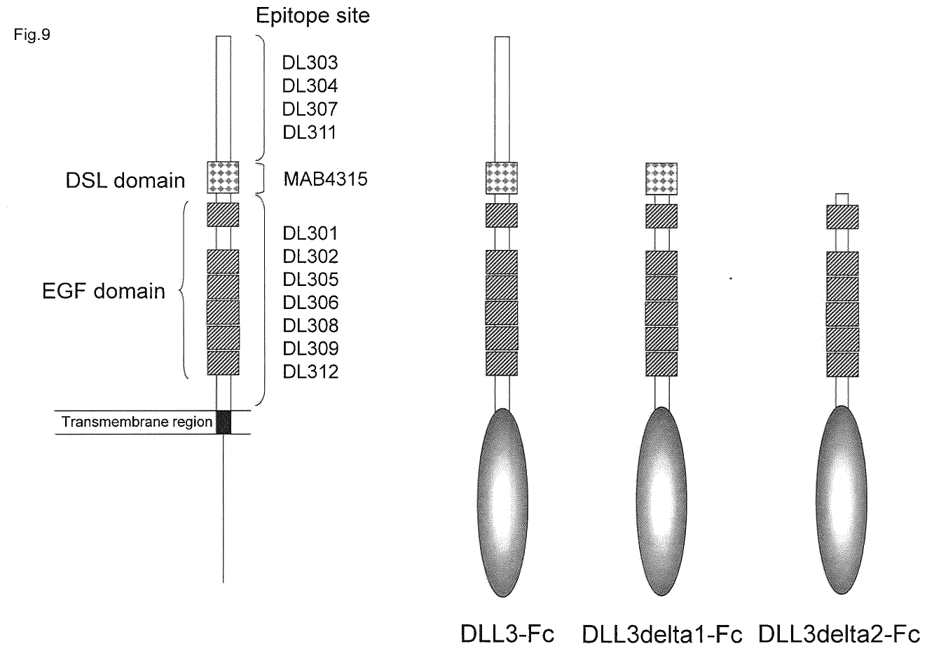

Human DLL3-Fc, mouse DLL3-Fc, human DLL3delta1-Fc, and human DLL3delta2-Fc were separately immobilized on a Nunc immunoplate (439454). Subsequently, the unreacted surface of the plate was blocked with a solution containing bovine serum albumin. After washing, each diluted antibody solution adjusted to an appropriate concentration was added thereto and incubated at room temperature for 1 hour. The reaction solution was removed from the plate. After washing with TBS containing Tween 20, alkaline phosphatase-labeled anti-mouse IgK antibodies were added to the plate and incubated for 1 hour. After washing of the plate, an alkaline phosphatase substrate Sigma 104 was added thereto and incubated at room temperature. After the incubation, the absorbance was measured at a wavelength of 405 nm and a reference wavelength of 655 nm. The results are shown in FIG. 2. All the purified antibodies bound to human DLL3-Fc in a dose-dependent manner. The monoclonal antibodies DL301, DL302, DL306, and DL312 and the commercially available antibody MAB4315 bound to mouse DLL3-Fc. The commercially available antibody MAB4315 bound to human DLL3delta1-Fc, but did not bind to human DLL3delta2-Fc, demonstrating that its epitope was located in the DSL domain. The DL303, DL304, DL307, and DL311 antibodies bound to neither human DLL3delta1-Fc nor human DLL3delta2-Fc. Thus, predicted epitopes for these antibodies are located between residues 27 and 175 in the amino acid sequence of SEQ ID NO: 1. DL301, DL302, DL305, DL306, DL308, DL309, and DL312 bound to both human DLL3delta1-Fc and human DLL3delta2-Fc, demonstrating that epitopes for these antibodies were located in residues 216-492 in the amino acid sequence of SEQ ID NO: 1. The schematic structure of the full-length DLL3 protein and the soluble DLL3-Fc fusion protein and a site recognized by each anti-DLL3 antibody are shown in FIG. 9.

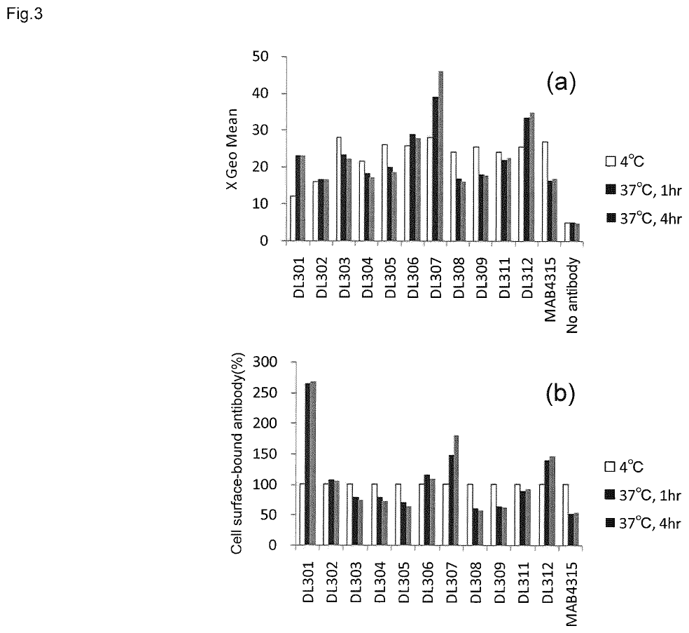

The binding of each monoclonal antibody to human DLL3 expressed on a cell membrane and the behavior of a human DLL3-monoclonal antibody complex on the cell membrane were analyzed by flow cytometry. Human DLL3/BaF3 cells were suspended in a FACS buffer (PBS containing 1% fetal bovine serum and 0.05% sodium azide). The cell suspension of 1×10⁶ cells/ml was reacted with the monoclonal antibody (final concentration: 5 μg/ml) at 4° C. for 30 minutes. After centrifugation and supernatant removal, the cells were washed once with a FACS buffer. FITC-labeled anti-mouse IgG (H+L) antibodies (Beckman Coulter, Inc.) were added thereto and incubated at 4° C. for 30 minutes. Unreacted FITC antibodies were removed by centrifugation. Then, the cells were resuspended and analyzed using a flow cytometer FACSCalibur (Becton, Dickinson and Company). For the purpose of analyzing the movement or disappearance of the DLL3-antibody complex from the cell membrane, human DLL3/BaF3 cells were suspended in an RPMI1640 culture medium containing 10% fetal bovine serum (FBS) and mouse IL3. The cell suspension of 1×10⁶ cells/ml was mixed with the monoclonal antibody (final concentration: 5 μg/ml) and reacted at 37° C. for 1 hour or 4 hours in a CO2 incubator. After the reaction, the amount of the antibody bound onto the cell membrane was analyzed by flow cytometry in the same way as above. The geometric mean of fluorescence intensity values (X Geo Mean) in a cell histogram plot was determined using the analytical software CELLQuest Pro included in FACSCalibur. All the isolated anti-DLL3 monoclonal antibodies bound to DLL3 on the cells (FIG. 3(a)). The reaction of the antibody with the cells at 4° C. inhibits membrane fluidity such as the cellular uptake of the DLL3-antibody complex from the cell membrane. The reaction of the antibody with the cells at 37° C. may cause the cellular uptake of the DLL3-antibody complex and shading (release from the cell membrane) resulting from protease digestion or the like. The amount of each monoclonal antibody bound onto the cell membrane as a result of incubation at 37° C. for 1 hour or 4 hours is shown in FIG. 3(b) as a relative value compared with that at 4° C. As a result of incubation at 37° C., the amount of the antibody DL303, DL304, DL305, DL308, DL309, or MAB4315 on the surface of the cell membrane was decreased. These results suggest the internalization or shading of the DLL3-antibody complex. By contrast, as a result of incubation at 37° C., the amount of the antibody DL301, DL306, DL307, DL311, or DL312 on the surface of the cell membrane was hardly changed or was increased, on the contrary. The latter result demonstrated that these antibodies were able to stably reside in the form of a DLL3 complex on the cell membrane.

The number of the DLL3 protein on cell surface was determined using a QIFIKIT (DAKO, F0479) for the quantitative determination of cell surface antigen by flow cytometry. The analysis was conducted with the anti-DLL3 antibody DL303 (final concentration: 5 μg/ml) as a primary antibody according to the protocol included therein. The numbers of the antigen on the surfaces of the human DLL3/BaF3, NCI-H1184 (ATCC), NCI-H1436 (ATCC), and Y79 (Riken Cell Bank) cells were approximately 9000, 7000, 6000, and 3000, respectively.

### [Example 4]Induction of ADCC Activity by Anti-DLL3 Antibody and Growth Inhibition Mediated by Antibody-Toxin Complex

Each anti-DLL3 antibody was examined for its antibody-dependent cell-mediated cytotoxicity (ADCC)-inducing activity against DLL3-expressing cells labeled with calcein. DLL3-expressing human DLL3/BaF3 and small-cell lung cancer NCI-H1184 (ATCC) cell lines were separately cultured for 90 minutes in the presence of 20 μg/ml Calcein-AM (Dojindo, 349-07201), then centrifuged, and washed to prepare calcein-labeled target cells. The target cells were seeded at 1×10⁴ cells/well to a 96-well plate (Coster 3799). Subsequently, the antibody adjusted to an appropriate final concentration was added thereto and incubated at room temperature for 15 minutes. Effector cells were added thereto at 5×10⁴ cells/well. The reaction plate was incubated at 37° C. in a CO2 incubator. The effector cells used were NK92 cells expressing a mouse FcγR3-human FcγR3 chimeric molecule (WO 2008/093688). After 4-hour incubation, the plate was centrifuged, and 100 μl of the culture supernatant was collected from each well. The fluorescence intensity was measured using ARVO SX (Wallac).

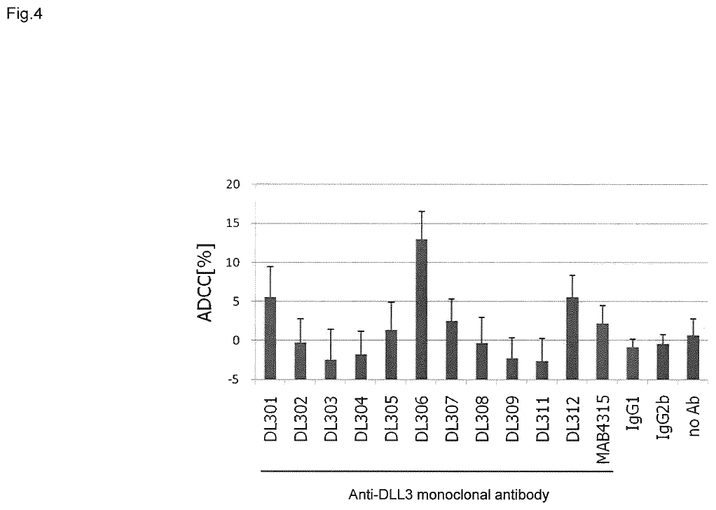

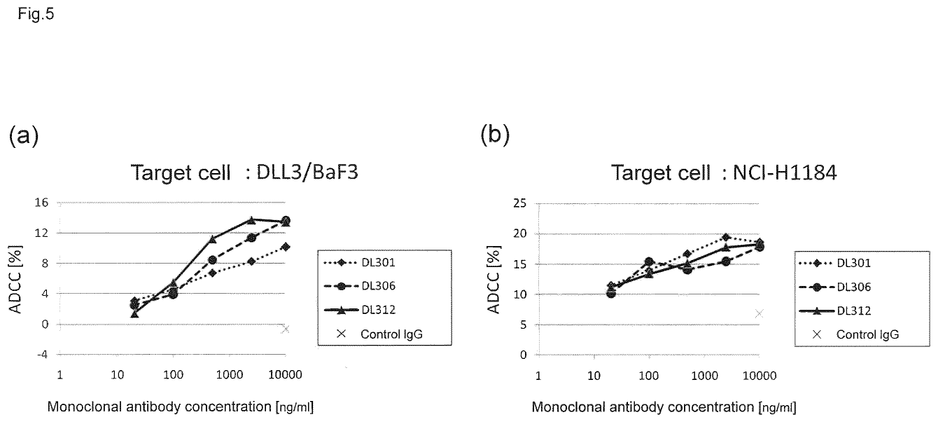

The ADCC-inducing activity was calculated according to the following formula:

$$ADCC [ \% ] = ( A - C ) / ( B - C ) \times 100 ,$$

wherein A represents fluorescence intensity in each well; B represents the mean of fluorescence intensity in the supernatant of cells lysed with Nonidet P-40 with the final concentration of 1%; and C represents the mean of fluorescence intensity in a well supplemented with only a medium. The mean and standard deviation were calculated from three measurements under each experimental condition. FIG. 4 shows the ADCC-inducing activity of the antibody added at the final concentration of 2.5 μg/ml against the DLL3/BaF3 cells. No ADCC activity was confirmed control antibodies IgG1 and IgG2b, whereas the ADCC-inducing activity was confirmed in DL301, DL306, and DL312. No distinct ADCC-inducing activity was confirmed in the commercially available monoclonal antibody MAB4315. FIG. 5 shows that the DL301, DL306, and D312 antibodies induce ADCC against DLL3/BaF3 and NCI-H1184 in a dose-dependent manner.

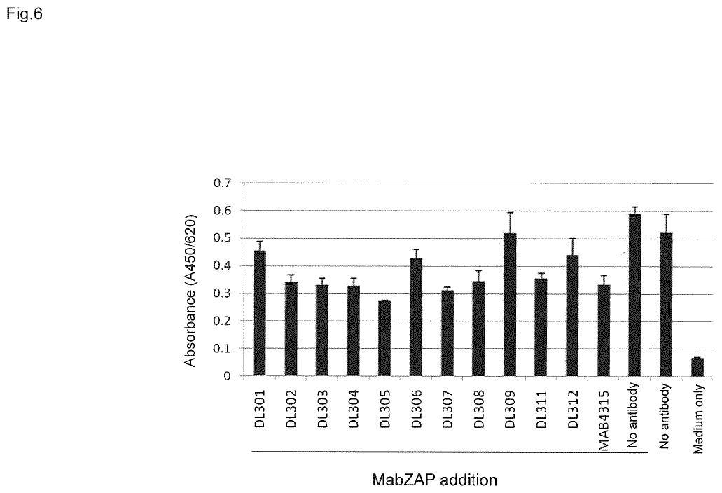

Each anti-DLL3 monoclonal antibody was evaluated for its cellular uptake using a toxin-labeled anti-mouse secondary antibody Mab-ZAP (Advanced Targeting Systems). DLL3/BaF3 cells were seeded at 5×10³ cells/well to a 96-well plate. Subsequently, the anti-DLL3 mouse monoclonal antibody and Mab-ZAP (final concentration: 1 μg/ml) were added thereto and incubated at 37° C. in a CO2 incubator. Four days later, Cell Count Reagent SF (Nacalai Tesque, Inc.) was added thereto. The absorbance was measured at 450 nm and at a control wavelength of 620 nm using a microplate reader to determine cell growth (FIG. 6). The addition of the anti-DLL3 monoclonal antibody and Mab-ZAP to the cells inhibited cell growth. The DL301, DL306, and DL312 antibodies confirmed to have an ADCC-inducing activity remained in large amounts on DLL3-expressing cells after being mixed with the cells and cultured at 37° C., and exhibited low growth inhibitory effect brought about by the cellular uptake of the Mab-ZAP complex.

### [Example 5]Cloning of Antibody Variable Region and Preparation of Recombinant Antibody

A Smart 5′-RACE cDNA library (Clontech Laboratories, Inc.) was prepared from the total RNAs of hybridomas producing each anti-DLL3 antibody. The total RNA preparation was performed using RNeasy Mini column (Qiagen). The cDNA library preparation followed the instruction of the manufacturer. Sequences encoding antibody variable regions (VH and VL) were amplified by PCR using primers complementary to sequences encoding antibody constant regions. The fragments thus amplified by PCR were cloned into pCR2.1TOPO and sequenced. Chimeric antibody expression vectors containing the obtained VH- and VL-encoding sequences and human IgG1 constant region-encoding sequences were constructed. The light chain- and heavy chain-encoding sequences were both incorporated in one expression vector for mammalian cells and transcribed under the control of a mouse CMV promoter. Table 1 shows SEQ ID NOs of the identified antibody variable region sequences and their CDR sequences, full-length mouse antibody sequences, and chimeric antibody sequences.

| | | | | | Variable |
|---|---|---|---|---|---|
| | | | | | region |
| | | CDR1 | CDR2 | CDR3 | sequence |
| [symbol] | [symbol] | NYLIE | VMNPGSGGTHYSEKFRG | SDYDYVTYAMDY | (SEQ ID |
| | | (SEQ ID NO: 12) | (SEQ ID NO: 13) | (SEQ ID | NO: 9) |
| | | | | NO: 14) | |
| [symbol] | [symbol] | KASQDINBYLI | RTNRLVD | LQYDEFPFT | (SEQ ID |
| | | (SEQ ID NO: 18) | (SEQ ID NO: 19) | (SEQ ID | NO: 15) |
| | | | | NO: 20) | |
| [symbol] | [symbol] | DYYMK | DINFNNGDTFYNQKFKG | DGNYAYFDY | (SEQ ID |
| | | (SEQ ID NO: 24) | (SEQ ID NO: 25) | (SEQ ID | NO: 21) |
| | | | | NO: 26) | |
| [symbol] | Light | RASKSVSTSGYSYMH | LASNLES | QHSRHLAWT | (SEQ ID |
| | chain | (SEQ ID NO: 30) | (SEQ ID NO: 31) | (SEQ ID | NO: 27) |
| | | | | NO: 32) | |
| DL309 | Heavy | NYYIE | EILPGSGSTTYNEKFKG | WGAREPGFPY | (SEQ ID |
| | chain | (SEQ ID NO: 36) | (SEQ ID NO: 37) | (SEQ ID | NO: 33) |
| | | | | NO: 38) | |
| | Light | KASQNVGTNVÁ | SASYRYS | QQYNNYFLT | (SEQ ID |
| | chain | (SEQ ID NO: 42) | (SEQ ID NO: 43) | (SEQ ID | NO: 39) |
| | | | | NO: 44) | |
| DL312 | Heavy | DYYMN | LIRNKANGYTTEYNASVKG | DSDGYYEYYFDY | (SEQ ID |
| | chain | (SEQ ID NO: 48) | (SEQ ID NO: 49) | (SEQ ID | NO: 45) |
| | | | | NO: 50) | |
| | Light | RASQEISDYLS | AASTLDS | LQYASYPYT | (SEQ ID |
| | chain | (SEQ ID NO: 54) | (SEQ TD NO: 55) | (SEQ ID | NO: 51) |
| | | | | NO: 56) | |
| | | Moose antibody | Chimeri antibody | | |
| | | sequence | sequence | | |

| | | | |
|---|---|---|---|
| DL301 | Heavy | (SEQ ID NO: 10) | (SEQ ID NO: 11) |
| | chain | | |
| | Light | (SEQ ID NO: 16) | (SEQ ID NO: 17) |
| | chain | | |
| DL306 | Heavy | (SEQ ID NO: 22) | (SEQ ID NO: 23) |
| | chain | | |
| | Light | (SEQ ID NO: 28) | (SEQ ID NO: 29) |
| | chain | | |
| DL309 | Heavy | (SEQ ID NO: 34) | (SEQ ID NO: 35) |
| | chain | | |
| | Light | (SEQ ID NO: 40) | (SEQ ID NO: 41) |
| | chain | | |
| DL312 | Heavy | (SEQ ID NO: 46) | (SEQ ID NO: 47) |
| | chain | | |
| | Light | (SEQ ID NO: 52) | (SEQ ID NO: 53) |
| | chain | | |

[symbol]

indicates data missing or illegible when filed

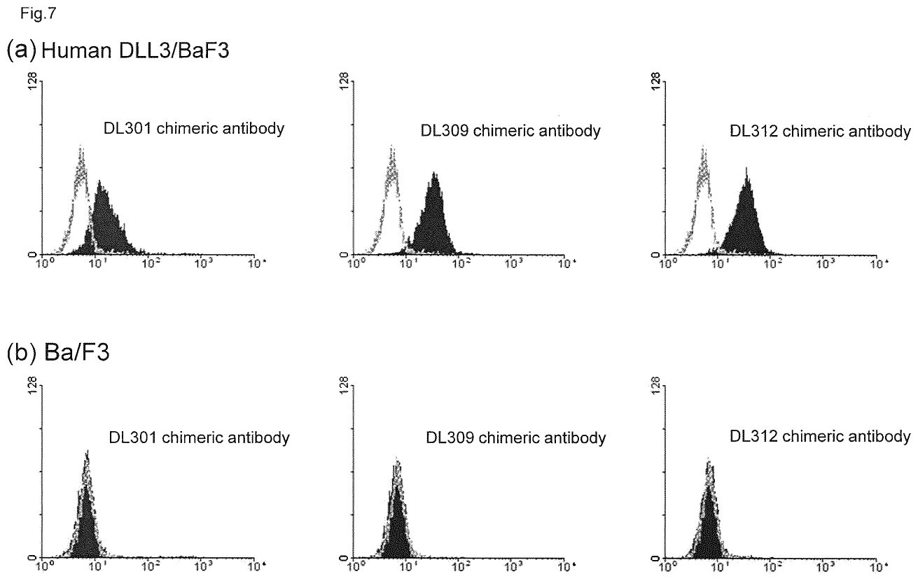

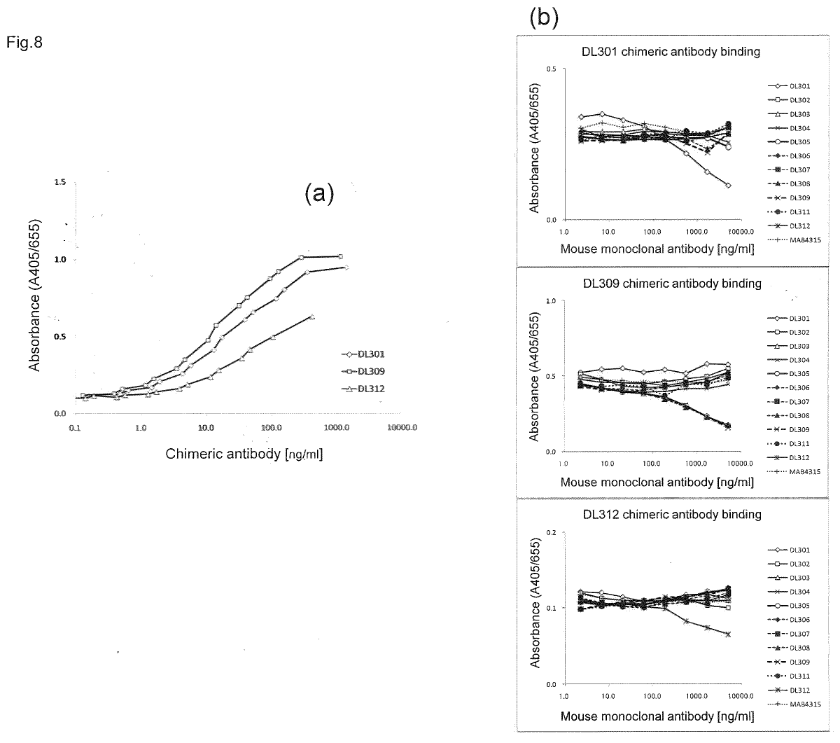

COS-7 cells were transfected with each chimeric antibody expression vector and allowed to transiently express the chimeric antibody. The chimeric antibody in the culture supernatant of COS-7 was confirmed to bind to the human DLL3 protein by flow cytometry and ELISA. The chimeric antibody concentration in the culture supernatant of COS-7 was calculated by sandwich ELISA. In this concentration calculation, a human chimeric antibody known for its concentration was used as a standard. The chimeric antibody (final concentration: 1 μg/ml) was added to human DLL3/BaF3 cells suspended in a FACS buffer (PBS containing 1% fetal bovine serum). After incubation at 4° C. for 30 minutes and washing, the cells were reacted with FITC-labeled anti-human antibodies (Beckman Coulter, Inc.), and the binding of the chimeric antibody was analyzed using a flow cytometer FACSCalibur. As shown in FIG. 7, the chimeric antibodies DL301, DL309, and DL312 bound to human DLL3/BaF3. None of the chimeric antibodies bound to Ba/F3 cells, which were a parental line of the forced expressing cells. Human DLL3-Fc was immobilized on a Nunc immunoplate, and the binding of each chimeric antibody to the immobilized protein was analyzed. For detecting antigen-bound chimeric antibodies, the chimeric antibody was added thereto, and alkaline phosphatase-labeled anti-human antibodies (Biosource, AHI0305) and an alkaline phosphatase substrate Sigma 104 were then added in this order. The absorbance was determined. The DL301, DL309, and DL312 chimeric antibodies bound to human DLL3-Fc in a dose-dependent manner (FIG. 8(a)).

### [Example 6]Grouping of Epitope by Competitive ELISA

The competition of a chimeric antibody and a mouse antibody for binding to the antigen molecule was analyzed by ELISA (FIG. 8 (b)). Human DLL3-Fc was added at 10 ng/well to a Nunc immunoplate and immobilized thereon. A mixture (final concentration: 50 ng/ml) of the chimeric antibody and the appropriately diluted mouse antibody was added to the DLL3-Fc protein-immobilized plate. The plate was incubated at room temperature for 1 hour and then washed. Alkaline phosphatase-labeled anti-human antibodies were added thereto and incubated. After the incubation, Sigma 104 was added thereto, and the absorbance was measured. The binding of the chimeric antibody to the antigen molecule competed with that of the original mouse monoclonal antibody thereto. The antibodies other than the DL301 mouse antibody did not inhibit the binding of the DL301 chimeric antibody to the antigen. The antibodies other than the DL312 mouse antibody did not inhibit the binding of the DL312 chimeric antibody to the antigen. These results demonstrated that DL301 and DL312 bound to their respective unique epitopes. The DL305, DL308, and DL309 mouse antibodies inhibited the binding of the DL309 chimeric antibody to the antigen at almost the same level, suggesting that an epitope was the same among these 3 antibodies. The DL306 mouse antibody confirmed to have an ADCC-inducing activity did not inhibit the binding of the DL301, DL309, or DL312 chimeric antibody to the antigen. These results demonstrated that DL306 recognized an epitope independent of that for DL301, DL309, or DL312.

### [Example 7]Establishment of Cell Line Expressing Recombinant Anti-DLL3 Antibody and Purification of Recombinant Antibody

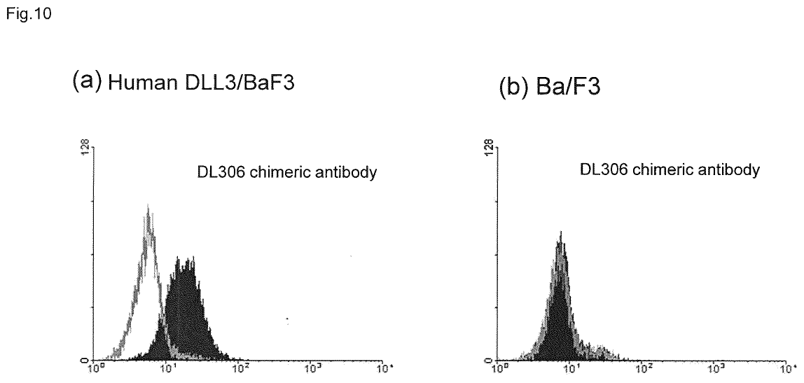

COS-7 cells were transfected with the DL306 chimeric antibody expression vector and allowed to transiently express the chimeric antibody. The chimeric antibody in the culture supernatant of COS-7 was confirmed to bind to the human DLL3 protein by flow cytometry. The chimeric antibody concentration in the culture supernatant of COS-7 was calculated by sandwich ELISA. In this concentration calculation, a human chimeric antibody known for its concentration was used as a standard. The chimeric antibody (final concentration: 1 μg/ml) was added to human DLL3/BaF3 cells suspended in a FACS buffer (PBS containing 1% fetal bovine serum). After incubation at 4° C. for 30 minutes and washing, the cells were reacted with FITC-labeled anti-human antibodies (Beckman Coulter, Inc.), and the binding of the chimeric antibody was analyzed using a flow cytometer FACSCalibur. As shown in FIG. 10, the chimeric antibody DL306 bound to human DLL3/BaF3 and did not bind to Ba/F3 cells, which were a parental line.

A cell line DG44 was transformed with the DL301, DL306, DL309, or DL312 human chimeric antibody expression vector. Drug-resistant cells were selected in the presence of Geneticin. Clones highly expressing the human chimeric antibody were selected by ELISA using anti-human antibodies to establish human chimeric antibody-producing DG44 cells.

Each protein of interest was purified from the culture supernatant of the human chimeric antibody-producing cell line by rProtein A affinity column chromatography and gel filtration chromatography. The concentration of the purified protein was determined by DC protein assay (Bio-Rad Laboratories, Inc.) with the IgG known for its concentration as a standard.

Mouse IgG2a antibody expression vector containing the VH- and VL-encoding sequences of DL301, DL306, or DL312 and mouse IgG2a constant region-encoding sequences were constructed. The light chain- and heavy chain-encoding sequences were both incorporated in one expression vector for mammalian cells and transcribed under the control of a mouse CMV promoter. A CHO cell line deficient in fucose transporter (WO2005/017155) was transformed with the mouse IgG2a antibody expression vector. Drug-resistant cells were selected in the presence of Geneticin. Clones highly expressing the mouse IgG2a antibody were selected by ELISA using anti-mouse IgG2a antibodies to establish mouse low-fucose IgG2a antibody-producing CHO cells. Each protein of interest was purified from the culture supernatant of the mouse low-fucose IgG2a antibody-producing cell line by Protein G affinity column chromatography and gel filtration chromatography. The concentration of the purified protein was determined by DC protein assay with the IgG known for its concentration as a standard.

TABLE 2

| | | |
|---|---|---|
| DL301 | Heavy chain | (SEQ ID NO: 69) |
| | Light chain | (SEQ ID NO: 16) |
| DL306 | Heavy chain | (SEQ ID NO: 70) |
| | Light chain | (SEQ ID NO: 28) |
| DL312 | Heavy chain | (SEQ ID NO: 71) |
| | Light chain | (SEQ ID NO: 52) |

### [Example 8]Induction of ADCC Activity by Recombinant Anti-DLL3 Antibody

Human chimeric and mouse IgG2a recombinant anti-DLL3 antibodies were examined for their antibody-dependent cell-mediated cytotoxicity (ADCC)-inducing activities against DLL3-expressing cells labeled with calcein. DLL3-expressing human DLL3/BaF3 and small-cell lung cancer NCI-H1184 (ATCC) cell lines were separately cultured for 90 minutes in the presence of 20 μg/ml Calcein-AM (Dojindo, 349-07201), then centrifuged, and washed to prepare calcein-labeled target cells. The target cells were seeded at 1×10⁴ cells/well to a 96-well plate (Coster 3799). Subsequently, the antibody adjusted to an appropriate final concentration was added thereto and incubated at room temperature for 15 minutes. Effector cells were added thereto at 5×10⁴ cells/well. The reaction plate was incubated at 37° C. in a CO2 incubator. The effector cells used for the recombinant mouse low-fucose IgG2a antibody were NK92 cells expressing a mouse FcγR4-human FcγR3 chimeric molecule (WO 2008/093688). The effector cells used for the human chimeric recombinant antibody were NK92 cells expressing a human FcγR3 molecule. After 4-hour incubation, the plate was centrifuged, and 100 μl of the culture supernatant was collected from each well. The fluorescence intensity was measured using ARVO SX. The ADCC-inducing activity was calculated by the method described in Example 4.

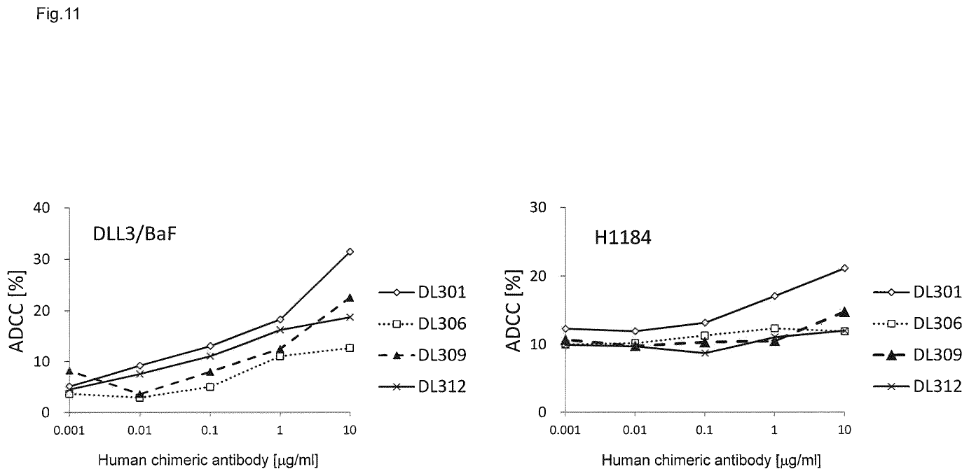

FIG. 11 shows that the recombinant human chimeric antibodies DL301, DL306, DL309, and D312 induce ADCC against DLL3/BaF3 and NCI-H1184 in a dose-dependent manner.

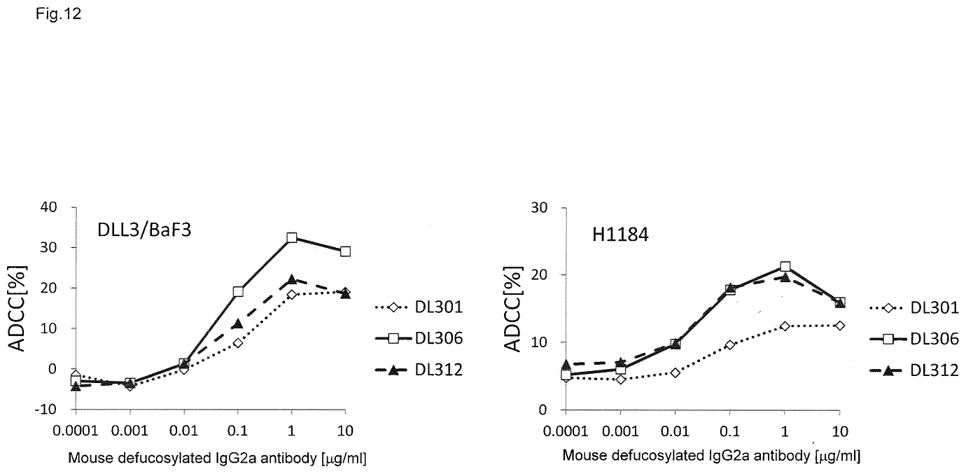

FIG. 12 shows that the recombinant mouse low-fucose IgG2a antibodies DL301, DL306, and D312 induce ADCC against DLL3/BaF3 and NCI-H1184 in a dose-dependent manner.

All publications, patents, and patent applications cited herein are incorporated herein by reference in their entirety.

1. An antibody which binds to DLL3 protein.

2. The antibody according to claim 1, wherein the antibody has a cytotoxic activity.

3. The antibody according to claim 2, wherein the cytotoxic activity is an antibody-dependent cell-mediated cytotoxic activity (ADCC activity).

4. The antibody according to claim 1, wherein the antibody has an internalization activity.

5. The antibody according to claim 1, wherein the antibody is conjugated with a cytotoxic substance.

6. The antibody according to claim 1, wherein the antibody recognizes a region from amino acids 216 to 492 in human DLL3 having the amino acid sequence as set forth in SEQ ID NO: 1.

7. An antibody which binds to DLL3 protein described in any of the following:
(1) an antibody comprising a heavy chain variable region comprising heavy chain CDR1 having the amino acid sequence as set forth in SEQ ID NO: 12, heavy chain CDR2 having the amino acid sequence as set forth in SEQ ID NO: 13, and heavy chain CDR3 having the amino acid sequence as set forth in SEQ ID NO: 14;
(2) an antibody comprising a heavy chain variable region comprising heavy chain CDR1 having the amino acid sequence as set forth in SEQ ID NO: 24, heavy chain CDR2 having the amino acid sequence as set forth in SEQ ID NO: 25, and heavy chain CDR3 having the amino acid sequence as set forth in SEQ ID NO: 26;
(3) an antibody comprising a heavy chain variable region comprising heavy chain CDR1 having the amino acid sequence as set forth in SEQ ID NO: 36, heavy chain CDR2 having the amino acid sequence as set forth in SEQ ID NO: 37, and heavy chain CDR3 having the amino acid sequence as set forth in SEQ ID NO: 38;
(4) an antibody comprising a heavy chain variable region comprising heavy chain CDR1 having the amino acid sequence as set forth in SEQ ID NO: 48, heavy chain CDR2 having the amino acid sequence as set forth in SEQ ID NO: 49, and heavy chain CDR3 having the amino acid sequence as set forth in SEQ ID NO: 50;
(5) an antibody comprising a light chain variable region comprising light chain CDR1 having the amino acid sequence as set forth in SEQ ID NO: 18, light chain CDR2 having the amino acid sequence as set forth in SEQ ID NO: 19, and light chain CDR3 having the amino acid sequence as set forth in SEQ ID NO: 20;
(6) an antibody comprising a light chain variable region comprising light chain CDR1 having the amino acid sequence as set forth in SEQ ID NO: 30, light chain CDR2 having the amino acid sequence as set forth in SEQ ID NO: 31, and light chain CDR3 having the amino acid sequence as set forth in SEQ ID NO: 32;
(7) an antibody comprising a light chain variable region comprising light chain CDR1 having the amino acid sequence as set forth in SEQ ID NO: 42, light chain CDR2 having the amino acid sequence as set forth in SEQ ID NO: 43, and light chain CDR3 having the amino acid sequence as set forth in SEQ ID NO: 44;
(8) an antibody comprising a light chain variable region comprising light chain CDR1 having the amino acid sequence as set forth in SEQ ID NO: 54, light chain CDR2 having the amino acid sequence as set forth in SEQ ID NO: 55, and light chain CDR3 having the amino acid sequence as set forth in SEQ ID NO: 56;
(9) an antibody comprising a heavy chain variable region comprising heavy chain CDR1 having the amino acid sequence as set forth in SEQ ID NO: 12, heavy chain CDR2 having the amino acid sequence as set forth in SEQ ID NO: 13, and heavy chain CDR3 having the amino acid sequence as set forth in SEQ ID NO: 14, and a light chain variable region comprising light chain CDR1 having the amino acid sequence as set forth in SEQ ID NO: 18, light chain CDR2 having the amino acid sequence as set forth in SEQ ID NO: 19, and light chain CDR3 having the amino acid sequence as set forth in SEQ ID NO: 20;
(10) an antibody comprising a heavy chain variable region comprising heavy chain CDR1 having the amino acid sequence as set forth in SEQ ID NO: 24, heavy chain CDR2 having the amino acid sequence as set forth in SEQ ID NO: 25, and heavy chain CDR3 having the amino acid sequence as set forth in SEQ ID NO: 26, and a light chain variable region comprising light chain CDR1 having the amino acid sequence as set forth in SEQ ID NO: 30, light chain CDR2 having the amino acid sequence as set forth in SEQ ID NO: 31, and light chain CDR3 having the amino acid sequence as set forth in SEQ ID NO: 32;
(11) an antibody comprising a heavy chain variable region comprising heavy chain CDR1 having the amino acid sequence as set forth in SEQ ID NO: 36, heavy chain CDR2 having the amino acid sequence as set forth in SEQ ID NO: 37, and heavy chain CDR3 having the amino acid sequence as set forth in SEQ ID NO: 38, and a light chain variable region comprising light chain CDR1 having the amino acid sequence as set forth in SEQ ID NO: 42, light chain CDR2 having the amino acid sequence as set forth in SEQ ID NO: 43, and light chain CDR3 having the amino acid sequence as set forth in SEQ ID NO: 44;
(12) an antibody comprising a heavy chain variable region comprising heavy chain CDR1 having the amino acid sequence as set forth in SEQ ID NO: 48, heavy chain CDR2 having the amino acid sequence as set forth in SEQ ID NO: 49, and heavy chain CDR3 having the amino acid sequence as set forth in SEQ ID NO: 50, and a light chain variable region comprising light chain CDR1 having the amino acid sequence as set forth in SEQ ID NO: 54, light chain CDR2 having the amino acid sequence as set forth in SEQ ID NO: 55, and light chain CDR3 having the amino acid sequence as set forth in SEQ ID NO: 56;
(13) an antibody comprising one or more amino acid substitutions, deletions, additions, and/or insertions in the antibody of any one of (1) to (12) and has an activity equivalent to that of any of the antibodies (1) to (12); and
(14) an antibody which binds to the same epitope as that in DLL3 protein to which any of the antibodies (1) to (12) bind.

8. A pharmaceutical composition comprising an antibody according to claim 1 as an active ingredient.

9. The pharmaceutical composition according to claim 8, wherein the pharmaceutical composition is an anticancer agent.

10. The pharmaceutical composition according to claim 9, wherein the anticancer agent targets lung cancer.

11. A method for diagnosing cancer, comprising the following steps:
(a) providing a sample isolated from a test subject; and
(b) detecting the expression level of DLL3 protein or DLL3 gene in the sample.

12. The diagnosis method according to claim 11, wherein the diagnosis method is intended for the diagnosis of lung cancer.

13. A diagnostic agent for cancer comprising an antibody according to claim 1.

14. A method of treatment for cancer comprising administering an antibody according to claim 1 as an active ingredient to a patient in need thereof.

15. The method according to claim 14, wherein the cancer is lung cancer.
